(19) United States
(12) Patent Application Publication
(10) Pub. No.: US 2024/0150772 A1
(43) Pub. Date: May 9, 2024

(54) NUCLEOLIN-TARGETING APTAMERS AND METHODS OF USING THE SAME

(71) Applicant: Duke University, Durham, NC (US)

(72) Inventors: Bruce A. Sullenger, Durham, NC (US); Michael Goldstein, Durham, NC (US); Elizabeth D. Pratico, Durham, NC (US); Michael Kastan, Durham, NC (US); Bethany Gray, Durham, NC (US)

(73) Assignee: Duke University, Durham, NC (US)

(21) Appl. No.: 18/205,390

(22) Filed: Jun. 2, 2023

Related U.S. Application Data
(62) Division of application No. 16/645,762, filed on Mar. 9, 2020, now Pat. No. 11,713,464.
(60) Provisional application No. 62/555,745, filed on Sep. 8, 2017.

(51) Int. Cl. C12N 15/115; A61P 35/00

(52) U.S. Cl. CPC C12N 15/115; A61P 35/00; C12N 2310/16

(57) ABSTRACT

Provided herein are compositions including aptamers capable of binding to and/or inhibiting the activity of nucleolin. Methods of treating cancer in a subject by administering such compositions are also provided.

### CROSS-REFERENCE TO RELATED APPLICATIONS

This patent application is a divisional of U.S. patent application Ser. No. 16/645,762, filed Mar. 9, 2020 which application is a national stage filing under 35 U.S.C. 371 of International Application No. PCT/US2018/050240, filed Sep. 10, 2018, which claims the benefit of priority of U.S. Provisional Patent Application No. 62/555,745, filed Sep. 8, 2017, all of which are incorporated herein by reference in their entirety.

### STATEMENT REGARDING FEDERALLY SPONSORED RESEARCH

This invention was made with government support by the National Institutes of Health under Award Number CA159826. The government has certain rights in the invention.

### SEQUENCE LISTING

This application is being filed electronically via Patent Center and includes an electronically submitted Sequence Listing in .xml format. The .xml file contains a sequence listing entitled “155554.00702.xml” created on Nov. 21, 2023 and is 658,560 bytes in size. The Sequence Listing contained in this .xml file is part of the specification and is hereby incorporated by reference herein in its entirety.

### INTRODUCTION

The protein nucleolin plays a critical role in repair of DNA double-stranded breaks (DSB) (Goldstein et al, PNAS, 2013). Mechanistically, nucleolin functions as a histone chaperone at the DSB, escorting the histone proteins H2A and H2B away from the nucleosome at the DNA break. This nucleosome disruption is required for the recruitment of repair enzymes and the repair of the DNA breaks. Therefore, inhibition of nucleolin results in sensitization of cells to DNA damaging agents. Importantly, the majority of human tumors overexpress nucleolin on the cell surface relative to normal cells, thus making nucleolin a tumor-preferential target. A nucleolin inhibitor would have the unique ability to specifically sensitize only tumor cells to DNA damaging agents as it should only target and internalize into cancerous cells.

Aptamers, small artificial RNA or DNA oligonucleotide ligands, can be selected to inhibit protein function and are also emerging as important tumor-targeting molecules. Additionally, they have many advantages over traditional antibody targeting agents, including ease of synthesis and amenability to chemical modification (Keefe et al, Nat Rev Drug Discov, 2010). Moreover, they exhibit antibody-like target affinities and specificities at a fraction of the size, allowing more efficient tumor penetration while maintaining the ability to discriminate between proteins that differ by only a few amino acids (reviewed in Conrad et al, Methods Enzymol, 1996; Obsorne et al, Chem Rev, 1997).

There is a need in the art for new aptamers that may bind to and/or inhibit the nucleolin protein. Such aptamers may be useful not only as new cancer treatments but also may facilitate the delivery of agents to the nucleus of a cell.

### SUMMARY

In one aspect of the present invention, aptamers are provided. The aptamer may include a polynucleotide having at least 50%, 60%, 70%, 80%, 85%, 90%, 91%, 92%, 93%, 94%, 95%, 96%, 97%, 98%, 99%, or 100% sequence identity to any one of SEQ ID NOS: 1-490, 494-515, or any one of the sequences described in the Tables or Figures disclosed herein (for example, Tables 1-4, 6-8 or FIG. 11A-11B, 12A-12B, 13A-13C, 14A-14D, 15A-15B, 16, 17, 18, 19, 20, 21, 22, 23, 24, 25, 26, 27, 28A-28B, 29, 30, 31, 32, 33, 34, 35, 36 or 37A-37B). In another aspect, the present invention relates to dimers, trimers, and tetramers including any one of the aptamers described herein.

In a further aspect of the present invention, pharmaceutical compositions including any of the aptamers described herein are provided. The pharmaceutical compositions may include a pharmaceutical carrier, excipient, or diluent.

In a still further aspect, the present invention relates to methods for treating cancer in a subject. The methods may include administering to the subject a therapeutically effective amount of any one of the aptamers, dimers, trimers, tetramers, or pharmaceutical compositions described herein.

In a still further aspect, methods of labeling or inhibiting nucleolin are provided. The methods include contacting nucleolin with any one of the compositions described herein to allow binding and possibly inhibition of the activity of the nucleolin. This contacting can be in vitro by adding the nucleolin to cells or may be in vivo by administering the compositions described herein to a subject. The compositions and aptamers provided herein are capable of binding to and possibly inhibiting the function of nucleolin.

### BRIEF DESCRIPTION OF THE DRAWINGS

FIG. 1 shows the work flow demonstrating the selection of aptamer families capable of relocating into the nucleus after binding to nucleolin on cell surface. A random 2′Fluoro-pyrimidine RNA pool of sequences GGGAGAGAGGAAGAGGGAUGGG (SEQ ID NO: 491)-N₄₀-CAUAACCCAGAGGUCGAUAGUACUGGAUCCCCCC (SEQ ID NO: 492) (where N₄₀ represents 40 random nucleotides) was incubated for 20 min at 37° C. with nucleolin protein (in 20 mM Hepes, 150 mM NaCl, 2 mM CaCl₂ and 0.01% bovine serum albumin) at ratios of RNA:protein varying from 187:1 to 133:1. RNA bound to protein was isolated by filtration through a 0.45 μm nitrocellulose membrane before RNA extraction, reverse transcription, PCR amplification and transcription to complete 1 round of selection. Each subsequent round of selection used the RNA pool transcribed from the previous round of selection, for a total of 7 rounds of SELEX against the nucleolin protein. The Round 6 RNA pool was also used to perform 2 Cell-SELEX rounds against both MCF-7 and Panc-1 cells. For the Cell-SELEX rounds, the Round 6 RNA pool was incubated with either MCF-7 or Panc-1 cells for 2 hrs at 37° C./5% CO2 before using a high salt wash to remove non-internalized RNA. Cells were then tryspinized, washed again with high salt, and RNA extracted from the cell nuclei using the Invitrogen™ PARIS™ kit. RNA pools from Rounds 3, 5, 7 and 9 (Panc-1) were reverse transcribed, PCR amplified and analyzed by High-Throughput Sequencing.

FIGS. 2A-2D show binding of the SELEX and Cell-SELEX rounds to the nucleolin protein (NCL). RNA pools from SELEX rounds 3, 6, and 7 or from Cell-SELEX Rounds 7-8 MCF-7 or Rounds 7-8 Panc-1 were end-labeled with ³²P. Nucleolin protein was serially diluted in 20 mM Hepes, 150 mM NaCl, 2 mM CaCl₂ and 0.01% bovine serum albumin and incubated with a trace amount of the ³²P-labeled RNA pools. After incubation at 37° C., unbound RNA was captured on a nylon membrane and RNA-nucleolin complexes captured on a nitrocellulose membrane. The fraction of protein-bound RNA was determined via phosphorimaging of the nitrocellulose and nylon membranes.

FIGS. 3A-3B show nucleolin-specific RNA aptamers bind to the RBD domain of nucleolin. (FIG. 3A) Map of truncated nucleolin mutants. From Chen et al. 2011, JBC. (FIG. 3B) Southwestern blot showing the binding of the initial RNA aptamer library (Sell) versus SELEX round 6 (R6 NCL) to truncated nucleolin mutants expressed in MCF7 cells.

FIG. 4 shows binding analysis of the nucleolin (NCL) aptamers identified through high throughput sequencing. Aptamers were end-labeled with ³²P. Nucleolin protein was serially diluted in 20 mM Hepes, 150 mM NaCl, 2 mM CaCl₂) and 0.01% bovine serum albumin and incubated with a trace amount of the ³²P-labeled RNA pools. After incubation at 37° C., unbound RNA was captured on a nylon membrane and RNA-nucleolin complexes captured on a nitrocellulose membrane. The fraction of protein-bound RNA was determined via phosphorimaging of the nitrocellulose and nylon membranes.

FIGS. 5A-5F show binding of nucleolin aptamer truncates to the nucleolin protein. Aptamers were end-labeled with ³²P. Nucleolin protein was serially diluted in 20 mM Hepes, 150 mM NaCl, 2 mM CaCl₂) and 0.01% bovine serum albumin and incubated with a trace amount of the ³²P-labeled RNA pools. After incubation at 37° C., unbound RNA was captured on a nylon membrane and RNA-nucleolin complexes captured on a nitrocellulose membrane. The fraction of protein-bound RNA was determined via phosphorimaging of the nitrocellulose and nylon membranes.

FIGS. 6A-6B show nucleolin specific RNA aptamer EV3 sensitizes colon cancer cells to ionizing radiation. HCT 116 p53−/− colon cancer cells were treated with 5 μg of indicated aptamers and exposed to 2Gy IR 48 h later. Cells were cultivated for 10d and survival was assessed by MTT assay.

FIG. 7 shows EV3 does not sensitize HFF (human foreskin fibroblasts), that do not express nucleolin on cell surface, to radiation. hTERT-immortalized HFF cells that do not express nucleolin on cell surface were treated with 5 μg of indicated aptamers and exposed to 2Gy IR 48 h later. Cells were cultivated for 10d and survival was assessed by MTT assay.

FIG. 8 shows EV3 and EV5 bind to nucleolin expressed on the cell surface in a concentration dependent manner. Flow cytometry analysis of MFI (mean fluorescence intensity) of DL650-labeled EV3 and EV5 after incubation of HCT116 p53−/− cells with the indicated aptamer concentrations.

FIGS. 9A-9D show binding of Ev3 aptamer truncates to the nucleolin protein. Aptamers were end-labeled with ³²P. Nucleolin protein was serially diluted in 20 mM Hepes, 150 mM NaCl, 2 mM CaCl₂) and 0.01% bovine serum albumin and incubated with a trace amount of the ³²P-labeled RNA pools. After incubation at 37° C., unbound RNA was captured on a nylon membrane and RNA-nucleolin complexes captured on a nitrocellulose membrane. The fraction of protein-bound RNA was determined via phosphorimaging of the nitrocellulose and nylon membranes.

FIG. 10 shows truncation of EV3 resulted in reduced activity as radiosensitizer. HCT 116 p53−/− colon cancer cells were treated with 5 ng of indicated full-length aptamers or EV3 truncates and exposed to 2Gy IR 48h later. Cells were cultivated for 10d and survival was assessed by MTT assay.

FIGS. 11A-11B show predicted secondary structures for a representative Family B aptamer (SEQ ID NO: 8).

FIGS. 12A-12B show predicted secondary structures for a representative Family C aptamer (SEQ ID NO: 9).

FIGS. 13A-13C show predicted secondary structures for a representative Family D aptamer (SEQ ID NO: 10).

FIGS. 14A-14D show predicted secondary structures for a representative Family E aptamer (SEQ ID NO: 11).

FIGS. 15A-15B show predicted secondary structures for a representative Family F aptamer (SEQ ID NO: 12).

FIG. 16 shows predicted secondary structures for Ev3min2 truncate aptamer (SEQ ID NO: 497).

FIG. 17 shows predicted secondary structures for Ev3min3 truncate aptamer (SEQ ID NO: 498).

FIG. 18 shows predicted secondary structures for Ev3min4 truncate aptamer (SEQ ID NO: 499).

FIG. 19 shows predicted secondary structures for Ev3min5 truncate aptamer (SEQ ID NO: 500).

FIG. 20 shows predicted secondary structures for Ev3min6 truncate aptamer (SEQ ID NO: 501).

FIG. 21 shows predicted secondary structures for Ev3min7 truncate aptamer (SEQ ID NO: 502).

FIG. 22 shows predicted secondary structures for Ev3min8 truncate aptamer (SEQ ID NO: 503).

FIG. 23 shows predicted secondary structures for Ev3min9 truncate aptamer (SEQ ID NO: 504).

FIG. 24 shows predicted secondary structures for Ev3min10 truncate aptamer (SEQ ID NO: 505).

FIG. 25 shows predicted secondary structures for Ev3min11 truncate aptamer (SEQ ID NO: 506).

FIG. 26 shows predicted secondary structures for Ev3min12 truncate aptamer (SEQ ID NO: 507).

FIG. 27 shows predicted secondary structures for Ev3min13 truncate aptamer (SEQ ID NO: 508).

FIGS. 28A-28B show predicted secondary structures for Ev3min14 truncate aptamer (SEQ ID NO: 509) and Ev3min15 truncate aptamer (SEQ ID NO: 510).

FIG. 29 shows predicted secondary structures for Ev3min16 truncate aptamer (SEQ ID NO: 511).

FIG. 30 shows predicted secondary structures for Ev3min17 truncate aptamer (SEQ ID NO: 512).

FIG. 31 shows predicted secondary structures for Ev3min18 truncate aptamer (SEQ ID NO: 513).

FIG. 32 shows predicted secondary structures for Ev3min19 truncate aptamer (SEQ ID NO: 514).

FIG. 33 shows predicted secondary structures for Ev3min20 truncate aptamer (SEQ ID NO: 515).

FIG. 34 shows predicted secondary structures for Ev3min21 truncate aptamer (SEQ ID NO: 486).

FIG. 35 shows predicted secondary structures for Ev3min22 truncate aptamer (SEQ ID NO: 487).

FIG. 36 shows predicted secondary structures for Ev3min23 truncate aptamer (SEQ ID NO: 488).

FIGS. 37A-37B show predicted secondary structures for Ev3min24 truncate aptamer (SEQ ID NO: 489) and Ev3min25 truncate aptamer (SEQ ID NO: 490).

### DETAILED DESCRIPTION

Here, in the non-limiting Examples, the present inventors disclose new aptamers that may bind to and/or inhibit the nucleolin protein. The present inventors demonstrate that such aptamers may be useful not only to sensitize cancer cells to cancer treatments including, for example, ionizing radiation and chemotherapeutic agents, but also may facilitate the delivery of agents to the nucleus of a cell.

In one aspect of the present invention, aptamers are provided. As used herein, the term “aptamer” refers to single-stranded oligonucleotides that bind specifically to target molecules with high affinity. Aptamers can be generated against target molecules, such as nucleolin, by screening combinatorial oligonucleotide libraries for high affinity binding to the target (See, e.g., Ellington, Nature 1990; 346: 8 18-22 (1990), Tuerk, Science 249:505-1 0 (1990)). The aptamers disclosed herein may be synthesized using methods well-known in the art. For example, the disclosed aptamers may be synthesized using standard oligonucleotide synthesis technology employed by various commercial vendors including, without limitation, Integrated DNA Technologies, Inc. (IDT), Sigma-Aldrich, Life Technologies, or Bio-Synthesis, Inc.

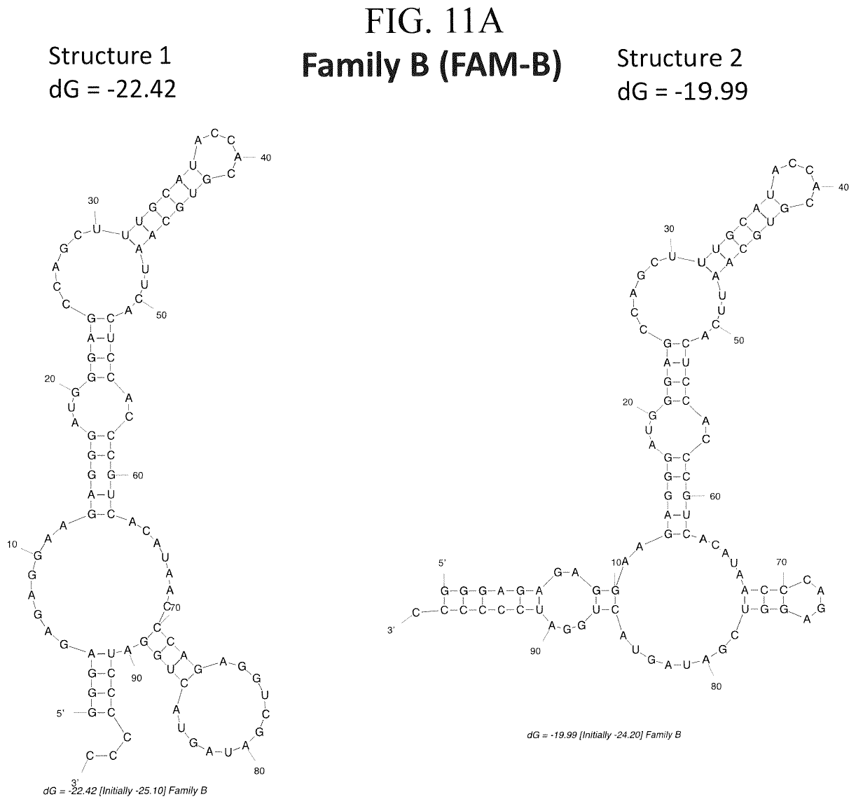

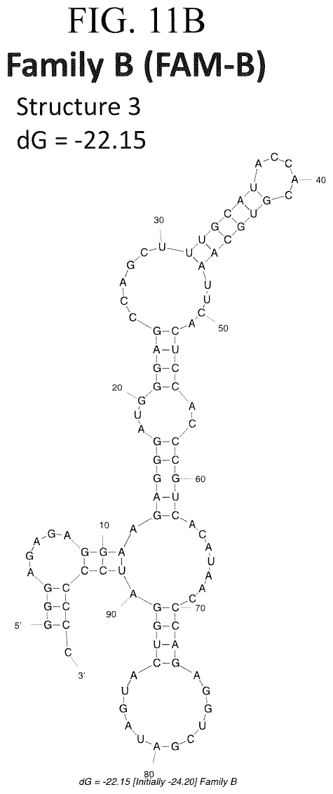

The aptamer may include a polynucleotide having at least 50%, 60%, 70%, 80%, 85%, 90%, 91%, 92%, 93%, 94%, 95%, 96%, 97%, 98%, 99%, or 100% sequence identity to any one of SEQ ID NOS: 1-490, 494-515, or any one of the sequences described in the Tables or Figures disclosed herein (for example, Tables 1-4, 6-8 or FIG. 11A-11B, 12A-12B, 13A-13C, 14A-14D, 15A-15B, 16, 17, 18, 19, 20, 21, 22, 23, 24, 25, 26, 27, 28A-28B, 29, 30, 31, 32, 33, 34, 35, 36 or 37A-37B). The aptamers described herein (i.e., SEQ ID NOS: 1-490, 494-515) may or may not include a 5′ constant region (GGGAGAGAGGAAGAGGGAUGGG (SEQ ID NO: 491)) that may be used, for example, to transcribe or purify the aptamers in vitro. The aptamers described herein (i.e., SEQ ID NOS: 1-490, 494-515) may or may not include a 3′ constant region (CAUAACCCAGAGGUCGAUAGUACUGGAUCCCCCC (SEQ ID NO: 492)) that may be used, for example, to transcribe or purify the aptamers in vitro. In some embodiments, the aptamer may include a polynucleotide having at least 50%, 60%, 70%, 80%, 85%, 90%, 91%, 92%, 93%, 94%, 95%, 96%, 97%, 98%, 99%, or 100% sequence identity to the polynucleotide sequence-5′-GGGAGAGAGGAAGAGGGAUGGG (SEQ ID NO: 491)-A Variable Region-CAUAACCCAGAGGUCGAUAGUACUGGAUCCCCCC (SEQ ID NO: 492)-3′, wherein the variable region may include any one of SEQ ID NOS: 13-473 or a portion thereof. The portion of the indicated aptamers should be capable of binding to nucleolin. In some embodiments, the aptamer may include a polynucleotide having at least 50%, 60%, 70%, 80%, 85%, 90%, 91%, 92%, 93%, 94%, 95%, 96%, 97%, 98%, 99%, or 100% sequence identity to SEQ ID NO: 480 (Ev3 Aptamer).

The terms “polynucleotide,” “nucleotide sequence,” “polynucleotide sequence,” “nucleic acid” and “nucleic acid sequence” refer to a nucleotide, oligonucleotide, polynucleotide (which terms may be used interchangeably), or any fragment thereof. These phrases may refer to DNA or RNA of genomic, natural, or synthetic origin.

Regarding polynucleotide sequences, the terms “sequence identity,” “percent identity,” and “% identity” refer to the percentage of base matches between at least two nucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Sequence identity for a nucleotide sequence may be determined as understood in the art. (See, e.g., U.S. Pat. No. 7,396,664). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known nucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website.

Regarding polynucleotide sequences, sequence identity is measured over the length of an entire defined nucleotide sequence, for example, as defined by a particular sequence identified herein. Furthermore, sequence identity, as measured herein, is based on the identity of the nucleotide base in the nucleotide sequence, irrespective of any further modifications to the nucleotide sequence. For example, the polynucleotide nucleotide sequences described herein may include modifications to the nucleotide sequences such 2′flouro, 2′O-methyl, and inverted deoxythymidine (idT) modifications. These modifications are not considered in determining sequence identity. Thus if a base, for example, is a 2′fluoro adenine (or 2′O-methyl, etc.), it is understood to be an adenine for purposes of determining sequence identity with another sequence. Likewise, 3′ idT modifications to the polynucleotide sequences described herein also should not be considered in determining sequence identity.

Based on the general aptamer structure presented, for example, in FIG. 11A-11B, 12A-12B, 13A-13C, 14A-14D, 15A-15B, 16, 17, 18, 19, 20, 21, 22, 23, 24, 25, 26, 27, 28A-28B, 29, 30, 31, 32, 33, 34, 35, 36 or 37A-37B, a person of ordinary skill in the art would readily recognize that several modifications could be made to the sequence while preserving the overall structure and presumably the function of the aptamer. For example, in FIG. 11A, a person of ordinary skill in the art could simply switch the first stem forming region GGGA and the tenth stem forming region UCCC to CCCU and AGGG, respectively, and still retain the stem structure of the aptamer. Additionally, modifications to the stem regions could be made that change the bases within the stem region but conserve the overall pyrimidine and purine base composition so that the stem region hybridizes at a similar melting temperature. A person of ordinary skill would also recognize that changes made to the aptamer that disturbed the general aptamer stem loop structure would likely result in an aptamer incapable of efficiently binding its target.

In some embodiments, the aptamer may have a dissociation constant (K_{D}) for the nucleolin protein that is less than 1000, 800, 600, 500, 450, 350, 250, 150, 125, 100, 90, 80, 70, 65, 60, 55, 50, 45, 40, 35, 30, 25, 20, 15, 10, 5, 2.5, 2, 1, 0.5, or 0.1 nanomolar (nM). The K_{D} of an aptamer may be measured using the methodology used by the inventors in the Examples.

The aptamers may include a polynucleotide (RNA, DNA, or peptide nucleic acid (PNA)) that is in an unmodified form or may be in a modified form including at least one nucleotide base modification. Nucleotide base modifications of polynucleotides to, for example, protect the polynucleotide from nuclease degradation and/or increase the stability of the polynucleotide and are well-known in the art. Common nucleotide base modifications that may be used in accordance with the present invention include, without limitation, deoxyribonucleotides, 2′-O-Methyl bases, 2′-Fluoro bases, 2′ Amino bases, inverted deoxythymidine bases, 5′ modifications, and 3′ modifications. In some embodiments, the aptamer may include a polynucleotide including a modified form including at least one nucleotide base modification selected from the group consisting of a 2′fluoro modification, a 2′O-methyl modification, a 5′ modification, and a 3′modification.

Typical 5′ modifications may include, without limitation, inverted deoxythymidine bases, addition of a linker sequence such as C6, addition of a cholesterol, addition of a reactive linker sequence which could be conjugated to another moiety such as a PEG. Typical 3′ modifications may include, without limitation, inverted deoxythymidine bases, and inverted abasic residues.

As additional 5′ and/or 3′ modifications, the aptamer may include a polynucleotide including a 5′ linker and/or a 3′ linker. Common 5′ and/or 3′ linkers for polynucleotides are known in the art and may include peptides, amino acids, nucleic adds, as well as homofunctional linkers or heterofunctional linkers. Particularly useful conjugation reagents that can facilitate formation of a covalent bond with an aptamer may comprise an N-hydroxysuccinimide (NHS) ester and/or a maleimide or using click chemistry. Typical 5′ and/or 3′ linkers for polynucleotides may include without limitation, amino C3, C4, C5, C6, or C12-linkers. The aptamer may further include an agent. Suitable agents may include, without limitation, stability agents, detectable agents such as reporter moieties, and/or therapeutic agents.

As used herein, a “stability agent” refers to any substance(s) that may increase the stability and/or increase the circulation time of a polynucleotide in vivo. Typical stability agents are known in the art and may include, without limitation, polyethylene glycol (PEG), cholesterol, albumin, or Elastin-like polypeptide.

As used herein, a “detectable agent” refers to any substance(s) that may be detected using appropriate equipment. Suitable detectable agents may be, without limitation, a fluorophore moiety, an enzyme moiety, an optical moiety, a magnetic moiety, a radiolabel moiety, an X-ray moiety, an ultrasound imaging moiety, a photoacoustic imaging moiety, a nanoparticle-based moiety, or a combination of two or more of the listed moieties.

A “fluorophore moiety” may include any molecule capable of generating a fluorescent signal. Various fluorophore moieties are well-known in the art and/or commercially available. Exemplary fluorophore moieties include, without limitation, fluorescein, FITC, Alexa Fluor 488, Alexa Fluor 660, Alexa Fluor 680, Alexa Fluor 750, and Alexa Fluor 790 (Life Technologies); Cy2, Cy3, Cy3.5, Cy5, Cy5.5 and Cy7 (GE Healthcare); DyLight 350, DyLight 488, DyLight 594, DyLight 650, DyLight 680, DyLight 755 (Life Technologies); IRDye 800CW, IRDye 800RS, and IRDye 700DX (Li-Cor); VivoTag680, VivoTag-S680, and VivoTag-S750 (PerkinElmer).

An “enzyme moiety” refers to polypeptides that catalyze the production of a detectable signal. Exemplary enzyme moieties may include, without limitation, horseradish peroxidase (HRP), alkaline phosphatase (AP), glucose oxidase, or β-galactosidase.

“Optical moieties” may include, for example, any agents that may be used to produce contrast or signal using optical imaging such as luminescence or acousto-optical moieties.

“Magnetic moieties” may include, for example, a chelating agent for magnetic resonance agents. Chelators for magnetic resonance agents can be selected to form stable complexes with paramagnetic metal ions, such as Gd(III), Dy(III), Fe(III), and Mn(II).

Other exemplary detectable agents may include radiolabel moieties. Exemplary radioactive labels may include, without limitation, ⁹⁹Mo, ^{99m}Tc, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ⁿC, ^{X}3N, ¹⁵O , and ¹⁸F.

“X-ray moieties” may include, for example, any agents that may be used to produce contrast or signal using X-ray imaging such as iodinated organic molecules or chelates of heavy metal ions.

“Photoacoustic imaging moieties” may include photoacoustic imaging-compatible agents such as methylene blue, single-walled carbon nanotubes (SWNTs), and gold nanoparticles. Ultrasound imaging moieties may include, for example, any agents that may be used to produce contrast or signal using ultrasound imaging such as Levovist, Albunex, or Echovist.

A detectable agent may also be a nanoparticle-based moiety. A nanoparticle-based moiety is a nanoparticle that is capable of generating a signal. For example, silicon containing nanoparticles may be used to produce fluoresecence, luminescence, or another type of signal. Other exemplary nanoparticle-based moieties include, without limitation, nanospheres such as Kodak X-SIGHT 650, Kodak X-SIGHT 691, Kodak X-SIGHT 751 (Fisher Scientific); metal oxide nanoparticles; and quantum dots such as EviTags (Evident Technologies) or Qdot probes (Life Technologies).

As used herein, a “therapeutic agent” may be any substance that provides a therapeutic functionality when conjugated to any one of the aptamers described herein. Suitable therapeutic agents may include, without limitation, cytotoxic compounds, and particularly those shown to be effective in other drug conjugates. As used herein, a “cytotoxic compound” refers to any substance that disrupts the functioning of cells and/or causes the death of cells. Various therapeutic cytotoxic compounds are known in the art and may include, without limitation, DNA damaging agents, anti-metabolites, natural products and their analogs. Exemplary classes of cytotoxic compounds include enzyme inhibitors such as dihydrofolate reductase inhibitors, and thymidylate synthase inhibitors, tubulin inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins, dolastatins, auristatins, maytansinoids, differentiation inducers, and taxols. More specifically, suitable cytoxic compounds may include: [symbol] [U6;1 [R 7:1 diphtheria toxin, botulinum toxin, geldanamycin, maytansinoids (including DM1), monomethylauristatin E (MMAE), monomethylauristatin F (MMAF), and maytansinoids (DM4) and their analogues3 Exemplary cytotoxic compounds may also include therapeutic radiopharmaceuticals including, without limitation ¹⁸⁶Re, ¹⁸⁸Re, ¹⁵³Sm, ⁶⁷Cu, ¹⁰⁵Rh, ^{m}Ag, and ¹⁹²Ir.

The aptamer and agent may be “linked” either covalently or non-covalently. Additionally, the aptamer and agent may be linked using the 5′ and/or 3′ linkers described herein. The aptamer and agent may be linked at the 5′ end and/or the 3′ end of the aptamer. To link the aptamer and agent non-covalently, the aptamer and the agent may be linked by a tag system. A “tag system” may include any group of agents capable of binding one another with a high affinity. Several tag systems are well-known in the art and include, without limitation, biotin/avidin, biotin/streptavidin, biotin/NeutrAvidin, or digoxigenin (DIG) systems. In some embodiments, the tag system comprises biotin/avidin or biotin/streptavidin. In such embodiments, the aptamer may be modified at either the 5′ or 3′ end to include biotin while the agent may be modified to include streptavidin or avidin. Alternatively, the aptamer may be modified at either the 5′ or 3′ end to include streptavidin or avidin while the agent may be modified to include biotin.

In another aspect, the present invention relates to dimers, trimers, and tetramers including any one of the aptamers described herein. A “dimer” refers to the linking together of two aptamer molecules in order to, for example, to increase the stability and/or increase the circulation time of a polynucleotide in vivo. A “trimer” refers to the linking together of three aptamer molecules in order to, for example, to increase the stability and/or increase the circulation time of a polynucleotide in vivo. A “tetramer” refers to the linking together of four aptamer molecules in order to, for example, to increase the stability and/or increase the circulation time of a polynucleotide in vivo. The aptamer molecules may be linked together covalently, noncovalently, or a combination of both. The aptamer molecules may be linked at their 5′ or 3′ ends. To link the aptamers noncovalently, the aptamers may be linked by a tag system or through a scaffold system.

In a further aspect of the present invention, pharmaceutical compositions including any of the aptamers described herein are provided. The pharmaceutical compositions may include a pharmaceutical carrier, excipient, or diluent (i.e., agents), which are nontoxic to the cell or mammal being exposed thereto at the dosages and concentrations employed. Often a pharmaceutical composition may include an aqueous pH buffered solution. Examples of pharmaceutical carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ brand surfactant, polyethylene glycol (PEG), and PLURONICS™ surfactant. In some embodiments, the pharmaceutical carrier may include a buffer including about 20 mM Hepes, pH 7.4; 150 mM NaCl; 1 mM CaCl₂); 1 mM MgCl₂; 5 mM KCl.

In a still further aspect, the present invention relates to methods for treating cancer in a subject. The methods may include administering to the subject a therapeutically effective amount of any one of the aptamers, dimers, trimers, tetramers, or pharmaceutical compositions described herein. The subject may be any mammal, suitably a human, domesticated animal such as a dog or cat, or a mouse or rat. Optionally, the present methods may further include administering a chemotherapeutic agent or radiation therapy to the subject.

Exemplary cancers in accordance with the present invention include, without limitation, colon, primary and metastatic breast, ovarian, liver, pancreatic, prostate, bladder, lung, osteosarcoma, pancreatic, gastric, esophageal, skin cancers (basal and squamous carcinoma; melanoma), testicular, colorectal, urothelial, renal cell, hepatocellular, leukemia, lymphoma, multiple myeloma, head and neck, and central nervous system cancers or pre-cancers.

Treating cancer includes, but is not limited to, reducing the number of cancer cells or the size of a tumor in the subject, reducing progression of a cancer to a more aggressive form, reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc.

Optionally, the present methods may further include administering a chemotherapeutic agent and/or radiation therapy to the subject. Without being limited by theory, the present inventors conjecture (and demonstrate in the Examples) that aptamers that block nucleolin function in cancer cells can sensitize cancer cells to DNA-damaging agents such as chemotherapeutic agents and radiation therapy. In some embodiments, the aptamer-containing composition described herein is administered prior to, simultaneously with, or after the chemotherapeutic agent and/or radiation therapy. In some embodiments, the aptamer-containing composition is administered prior to the administration of the optional chemotherapeutic agent and/or radiation therapy.

Chemotherapeutic agents are compounds that may be used to treat cancer. Suitable chemotherapy agents [symbol] capecitabine, floxuridine, and gemcitabine, and the purine analogs 6-mercaptopurine, 8-azaguanine, fludarabine, and cladribine. The optional radiation therapy in the present methods may include one or more doses of between 1 Gy and 30 Gy. Suitably, the radiation therapy includes a single fraction dose of 12, 15, 18, 20, 21, 23, 25, or 28 Gy.

The chemotherapeutic agent and/or radiation therapy may be administered in any order in relation to the aptamer-containing compositions described herein, at the same time or as part of a unitary composition. The aptamer-containing composition and chemotherapeutic agent and/or radiation therapy may be administered such that one composition or therapy is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks or more.

An “effective amount” or a “therapeutically effective amount” as used herein means the amount of a composition that, when administered to a subject for treating a state, disorder or condition is sufficient to effect a treatment (as defined above). The therapeutically effective amount will vary depending on the composition, formulation or combination, the disease and its severity and the age, weight, physical condition and responsiveness of the subject to be treated. The compositions (i.e., those including the aptamers described herein) described herein may be administered by any means known to those skilled in the art, including, but not limited to, intratumoral, oral, topical, intranasal, intraperitoneal, parenteral, intravenous, intramuscular, subcutaneous, intrathecal, transcutaneous, nasopharyngeal, or transmucosal absorption. Thus the compositions may be formulated as an ingestable, injectable, topical or suppository formulation. Within broad limits, administration of larger quantities of the aptamer-containing compositions is expected to achieve increased beneficial biological effects than administration of a smaller amount. Moreover, efficacy is also contemplated at dosages below the level at which toxicity is seen.

It will be appreciated that the specific dosage administered in any given case will be adjusted in accordance with the aptamer-containing compositions being administered, the disease to be treated or inhibited, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations.

The maximal dosage for a subject is the highest dosage that does not cause undesirable or intolerable side effects. The number of variables in regard to an individual prophylactic or treatment regimen is large, and a considerable range of doses is expected. The route of administration will also impact the dosage requirements. It is anticipated that dosages of the compound will reduce symptoms of the condition at least 10%, 20%, 30%, 40%, 50%, 60%, 70%, 80%, 90% or 100% compared to pre-treatment symptoms or symptoms is left untreated. It is specifically contemplated that pharmaceutical preparations and compositions may palliate or alleviate symptoms of the disease without providing a cure, or, in some embodiments, may be used to cure the disease or disorder.

The effectiveness of the aptamer-containing composition in treating the cancer or reducing the likelihood of resistance can be measured by tracking the growth of the tumor or the growth rate of the tumor or cancer cells. A decrease in tumor size or in the rate of tumor growth is indicative of treatment of the cancer.

The aptamers disclosed herein may also be used in methods of labeling or inhibiting nucleolin. As disclosed herein the aptamers provided bind to nucleolin and may be used to inhibit nucleolin. In some instances the aptamers are trafficked with the nucleolin to the nucleus of the cell when the aptamer is contacts the cell. The aptamers may be combined with an agent as described above and if the agent is a reporter moiety the agent may allow nucleolin to be labeled within the cell or to bring the agent in contact with nucleolin. Nucleolin may be contacted with the aptamer directly or indirectly in vivo, in vitro, or ex vivo. Contacting encompasses administration to a cell, a culture of cells, tissue, mammal, patient, or human expressing nucleolin. Further, contacting a cell includes adding an agent to a cell culture. Other suitable methods may include introducing or administering an agent to a cell, tissue, mammal, or patient using appropriate procedures and routes of administration as defined above.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1—Development of Nucleolin-Binding Aptamers

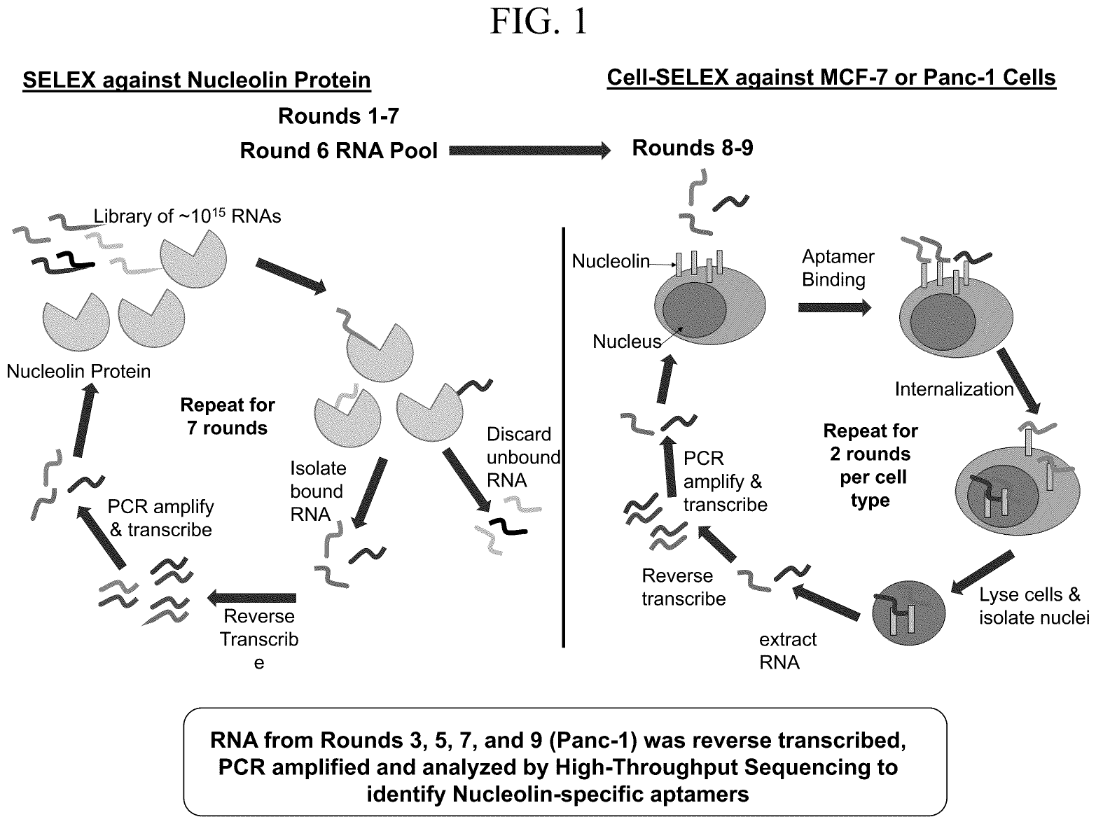

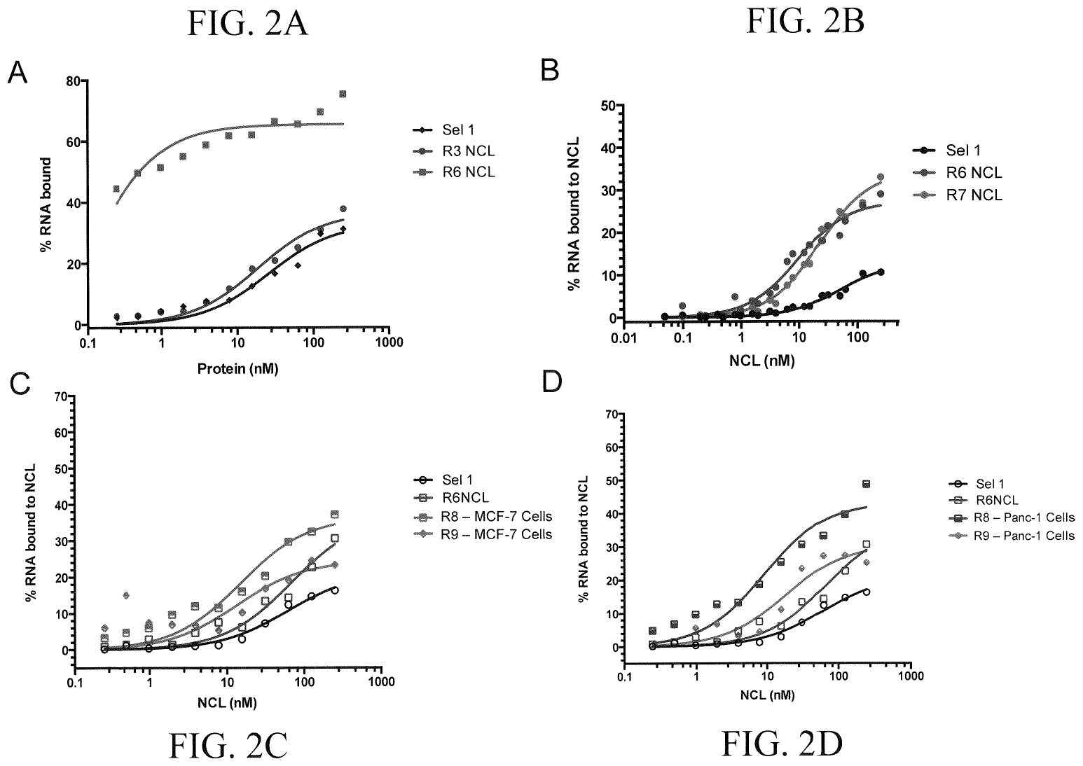

With the goal of developing an aptamer that binds and/or inhibits the nucleolin protein, we performed a dual protein and cell selection via systematic evolution of ligands by exponential enrichment (SELEX) using a modified RNA library (FIG. 1). First, SELEX was performed against a recombinant nucleolin protein (SEQ ID NO: 493) resulting in an RNA library enriched in clones specific for nucleolin after 6 rounds of selection (FIG. 2A). As a 7^{th} round of SELEX did not improve the aptamer pool's affinity for the nucleolin protein (FIG. 2B), we moved forward with the pool of RNA from the 6^{th} round of SELEX (R6 NCL). To identify nucleolin-specific RNAs capable of binding to nucleolin on cell surface and subsequently transporting to the nucleus, the R6 NCL RNA pool was incubated with either MCF-7 or Panc-1 cells (FIG. 1). The nuclei were then isolated and the aptamer pool that reached this compartment was amplified. After 2 rounds of cellular selection with either MCF-7 or Panc-1 cells, the RNA library was further enriched for aptamers capable of binding to the nucleolin protein (FIGS. 2C & 2D).

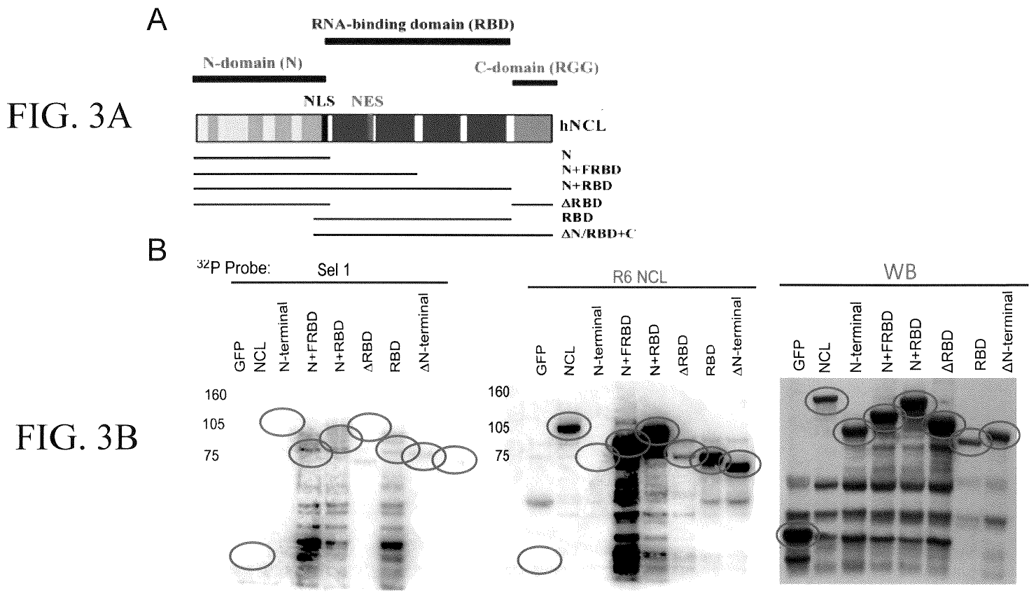

We previously demonstrated that nucleolin interacts with Rad50, a member of the MRN complex, through its C-terminal RGG domain and that this interaction is essential for recruitment of nucleolin to the DNA damage site and repair of the DSB (Goldstein et al. 2013, PNAS). Thus, we estimated that in order to achieve a disruption of the nucleolin-Rad50 interaction and the inhibition of DSB repair required for radiosensitization, our nucleolin aptamer would need to bind to either the RGG domain itself or to the RBD domain in the proximity of the C-terminus. In fact, we found that the R6 NCL RNA aptamer pool binds to the RBD domain (FIGS. 3A-3B), suggesting that these aptamers may be able to inhibit the nucleolin-Rad50 interaction that is crucial for DSB repair.

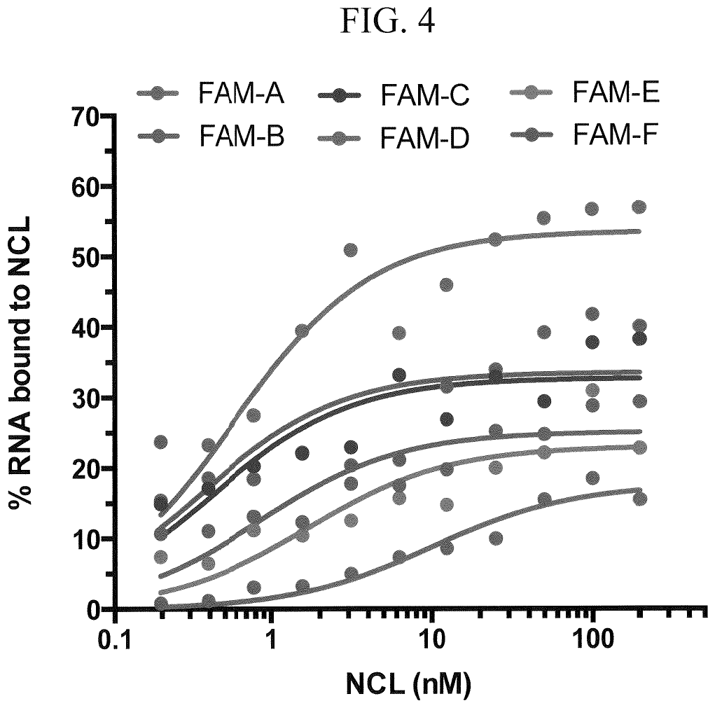

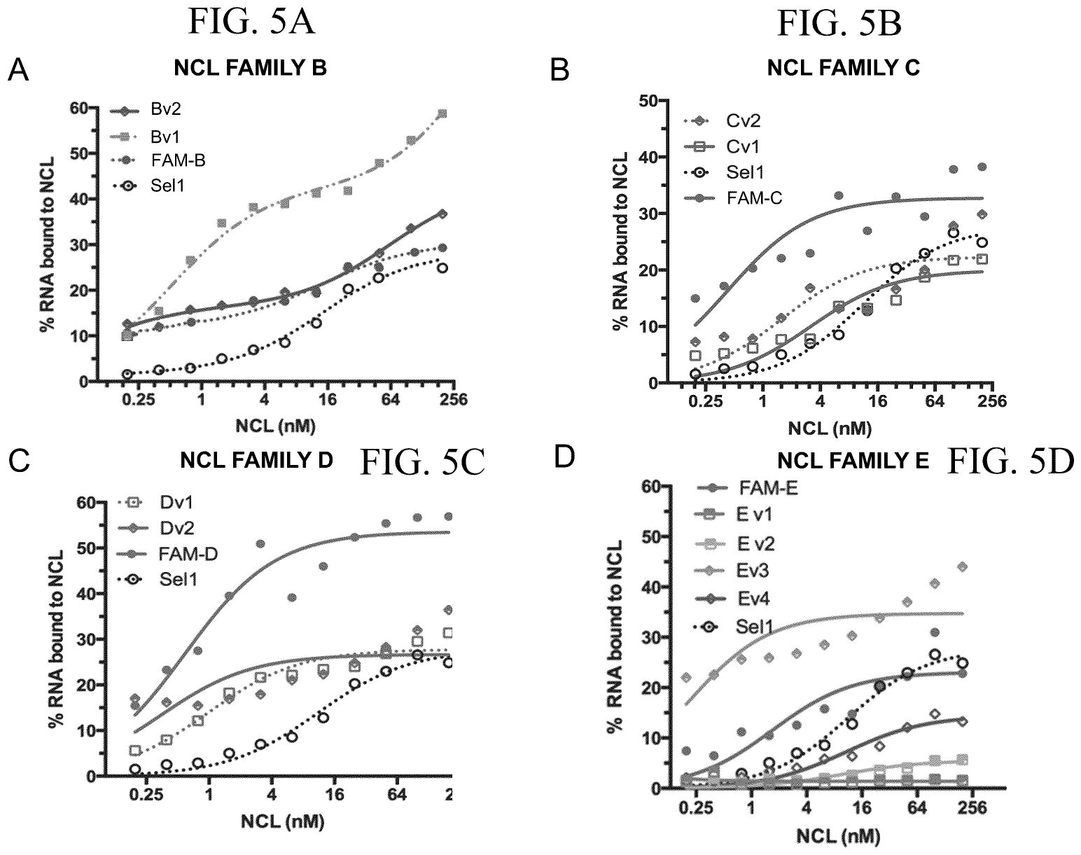

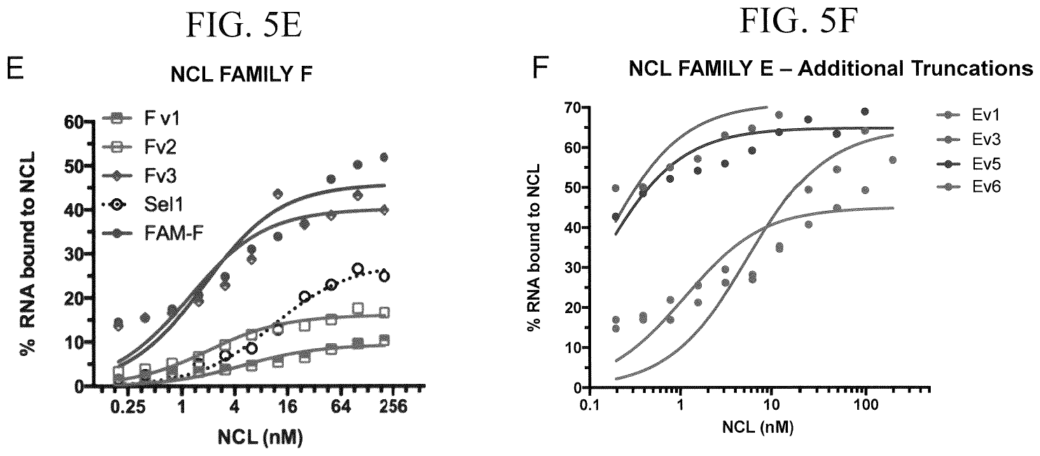

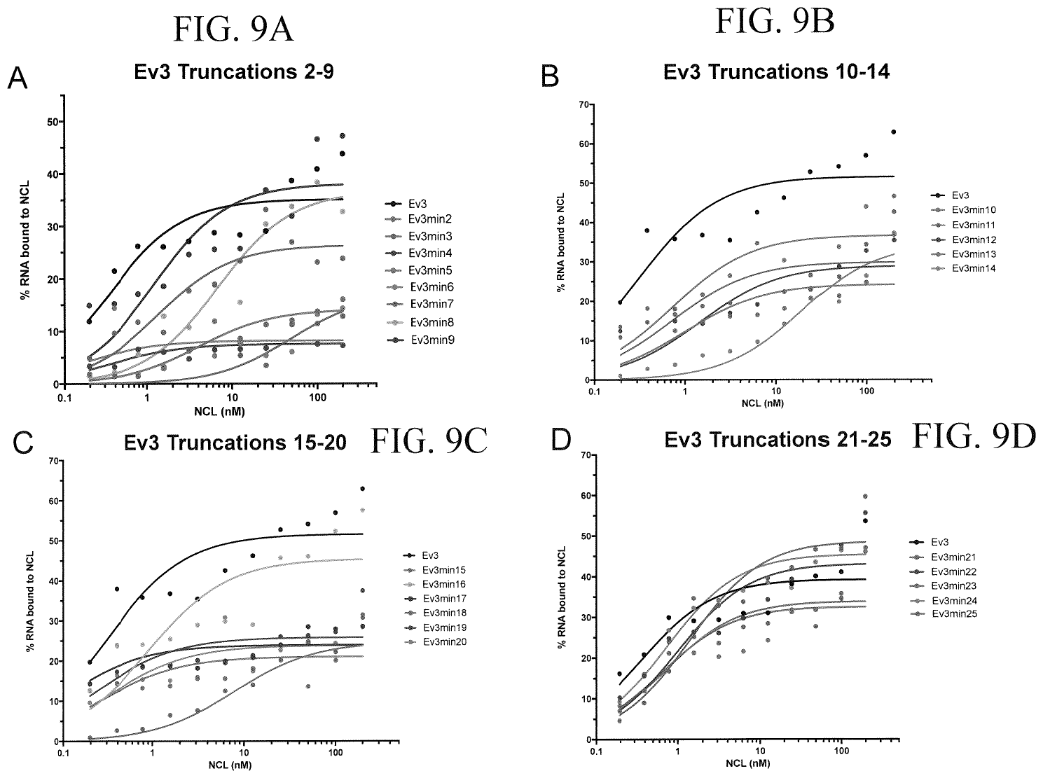

High throughput sequencing of the SELEX pools from various selection rounds (rounds 3, 5, 7, and 9—Panc-1 round 2), resulted in almost 8000 unique RNA families plus 78 ambiguous sequences, where RNA families are RNA sequences that differ by 4 nucleotides or less and ambiguous sequences are single RNA sequences that do not fit into a RNA family. The most representative sequence from each of the top 6 abundant families, designated Families A-F (FAM-A, etc., Tables 1-4), were transcribed to test their ability to bind to the nucleolin protein. Families B-F demonstrated specific binding to nucleolin while Family A did not appear to significantly bind the protein, suggesting that it may be an artifact resulting from PCR amplification (FIG. 4, Table 5). To make it easier to chemically synthesize the nucleolin aptamers, we sought to shorten their length. Thus, we designed truncates of the Families B-F aptamers (Tables 6-8). Several of these truncations resulted in improved affinity for nucleolin over the parent aptamers, with truncations Bv1, Dv2, Ev3, Ev5, and Fv3 demonstrating the best affinity (FIGS. 5A-5F). To further truncate the Ev3 aptamer, we designed 24 additional truncates of Ev3 (Tables 7 and 8). Several of these truncations, primarily Ev3.min21, Ev3.min22, and Ev3.min24 demonstrated a similar affinity for nucleolin compared to their parent Ev3 aptamer (FIGS. 9A-9D).

TABLE 1
Nucleolin Aptamer Sequences without 5′ and 3′ Constant Regions

| NCL | |
|---|---|
| Aptamer | Sequence |
| FAM-A | CCAUCUAGAUCUCCGUAGAUUCCCCCGGCUCUUUCUCGC (SEQ ID NO: |
| | 1) |
| FAM-B | AGCCAGCUUUGCAUACCACGUGCAAUUCACUCCACCCGUCA (SEQ ID |
| | NO: 2) |
| FAM-C | AAGAUCUGCUAAGUGCACGCACAAUCACCAUCGAGCGUCU (SEQ ID NO: |
| | 3) |
| FAM-D | CACAUGGUACGCCCAAAGCGAGGCCCGCUGCGUAGUGC (SEQ ID NO: 4) |
| FAM-E | CACGGUCCAGCGCUAACUGUACCUGCUGUGCCACCCACCG (SEQ ID NO: |
| | 5) |
| FAM-F | ACCACGCGCCAACGUGUCAGCUACACGCCGUGUUCCCCGG (SEQ ID NO: |
| | 6) |

TABLE 2
Nucleolin Aptamer Sequences with 5′ and 3′ Constant Regions

| NCL | |
|---|---|
| Aptamer | Sequence |
| FAM-A | GGGAGAGAGGAAGAGGGAUGGGCCAUCUAGAUCUCCGUAGAUUCCCC |
| | CGGCUCUUUCUCGCCAUAACCCAGAGGUCGAUAGUACUGGAUCCCCCC |
| | (SEQ ID NO: 7) |
| FAM-B | GGGAGAGAGGAAGAGGGAUGGGAGCCAGCUUUGCAUACCACGUGCAA |
| | UUCACUCCACCCGUCACAUAACCCAGAGGUCGAUAGUACUGGAUCCCC |
| | CC (SEQ ID NO: 8) |
| FAM-C | GGGAGAGAGGAAGAGGGAUGGGAAGAUCUGCUAAGUGCACGCACAAU |
| | CACCAUCGAGCGUCUCAUAACCCAGAGGUCGAUAGUACUGGAUCCCCC |
| | C (SEQ ID NO: 9) |
| FAM-D | GGGAGAGAGGAAGAGGGAUGGGCACAUGGUACGCCCAAAGCGAGGCC |
| | CGCUGCGUAGUGCCAUAACCCAGAGGUCGAUAGUACUGGAUCCCCCC |
| | (SEQ ID NO: 10) |
| FAM-E | GGGAGAGAGGAAGAGGGAUGGGCACGGUCCAGCGCUAACUGUACCUG |
| | CUGUGCCACCCACCGCAUAACCCAGAGGUCGAUAGUACUGGAUCCCCC |
| | C (SEQ ID NO: 11) |
| FAM-F | GGGAGAGAGGAAGAGGGAUGGGACCACGCGCCAACGUGUCAGCUACA |
| | CGCCGUGUUCCCCGGCAUAACCCAGAGGUCGAUAGUACUGGAUCCCCC |
| | C (SEQ ID NO: 12) |

TABLE 3
Representative Nucleolin Aptamer Sequences without 5′ and 3′
Constant Regions from All Families

| Family | | SEQ ID |
|---|---|---|
| Name | Representative Sequence | NO: |
| A | CCAUCUAGAUCUCCGUAGAUUCCCCCGGCUCUUUCUCGC | 13 |
| B | AGCCAGCUUUGCAUACCACGUGCAAUUCACUCCACCCGUCA | 14 |
| C | AAGAUCUGCUAAGUGCACGCACAAUCACCAUCGAGCGUCU | 15 |
| D | CACAUGGUACGCCCAAAGCGAGGCCCGCUGCGUAGUGC | 16 |
| E | CACGGUCCAGCGCUAACUGUACCUGCUGUGCCACCCACCG | 17 |
| F | ACCACGCGCCAACGUGUCAGCUACACGCCGUGUUCCCCGG | 18 |
| G | AAGAUCCUCGCGCAUCUGCCGAGCAAUCACCAUCGGACG | 19 |
| H | CCAAAUGCCAAGCCGUAGCCCGGCCAGUAGCCCACACGUC | 20 |
| I | UGCCAAGCCGAGGCCCGGCCACCAUCCACUGAUAGUGGGC | 21 |
| J | AAGAUCCUGACGCGACACAGCAAUCACCAUCGAACCAGCU | 22 |
| K | AAGAUCUGCGGCAACGCACAAUCACCAUCGAUUCCGAAUU | 23 |
| L | GAGCUCUCGAUUUCCUCCGCGACACCCAUCCAAACCUCA | 24 |
| M | CUCUCCGGUCUACCAUCCGGACCGGCGACAAAGUCAACUU | 25 |
| N | AAGAUCUGCUAUGCACAAUCACCAUCGGGCGCUCCGGGGAA | 26 |
| O | UUGACUCUGCUGCGUAGUUCGCACCAAGAUCAACCACUUC | 27 |
| P | UACCAAGUCGUGGCCCGACUACCCAGCACGAUGCGCAA | 28 |
| Q | CUAUUCGAGUUCCCACGAAUCCCCCCAUCGAGAACCUAC | 29 |
| R | UGCCAAGCCGAGGCCCGGCCACCGUCCCCGCGGCUGAUGA | 30 |
| S | AAUGAUCUCGCCAAUGGGCGACAAUCACCAUGUCUUCACA | 31 |
| T | UCAGUGCGCCAAGUGGAGGCCCCACCGCAGCCCAUCAA | 32 |
| U | UGUAUGCCAGCUUUGACGAUAACUGUCGCGCGUCAAUUCA | 33 |
| V | UACGCCAAAGUGGAGCCCACUCGUACCCCAUCAUGAGCUG | 34 |
| W | CCGCCAGCUUUGGGUACCCUGACCAAUUCACGGCCAUCCA | 35 |
| X | GUAAUUGUCUGAGACCACCGGACAAUCAACAAGAAAUCCU | 36 |
| Y | UCAGGCCAAAGUGUGAUAGCCACACCCGCACCCAUCAGGA | 37 |
| Z | CCGACCGCCGACCAGGGUGCCACUCGUACCCCUGUCCGCC | 38 |
| AA | UGCCAAGUCGAAGCCCGACCACGCCAUCCCUAACAGUGCC | 39 |
| AB | ACUUGUGCUGAGUCGCCAAAGUGAGGCCCACUCGCCAGCA | 40 |
| AC | CCGCCAGCUCCUCUGAGGCACAAGAGGUUCACGGUGAUCC | 41 |
| AD | CACCAGGUUCUGCUGUCCCCAAGCGCUGACCCAUCCUUCC | 42 |
| AE | AAGAUCCGGUAACUCCCCACCGCAAUCACCGUCGACUACU | 43 |
| AF | CCAUCUAGAUCUCCGUAGAUUCCCCCCGGCUCUUUCUCGC | 44 |
| AG | CCAUCUGAACCCACAGAUUCCCCCAUCAUCAGCCACAGUG | 45 |
| AH | CACUAAGUUGGUAGCCCCAACUGCCCCGACACGAGGAUGU | 46 |
| AI | UUGUGCUCCGUGGCUCCCCGGACCAACCGCUUCCAGCAGU | 47 |
| AJ | CAAUCACGCGUAGUACGUCGCGGAAGAUCCCCAUGCCGA | 48 |
| AK | CACAUGGUACGCCCAAAAGCGAGGCCCGCUGCGUAGUGC | 49 |
| AL | UGCCAUACGCGGUUCGAAGUCGAAGCCCGACAACCCGGCA | 50 |
| AM | GUUAUUCACAUGCCUCCCGUGAAUCAACAAGAAUUCCUUG | 51 |
| AN | AAAGAUCUAGACUGUAAGUCUCCAAUCGCCCAGUUAAUUC | 52 |
| AO | GCCCAAUCGCCAGUGGAACGCGCUGAAGGAUCUGCACCC | 53 |
| AP | UGCAACGUAAAAGAGAGUCAUCUCAGGCUAGUCGUCUACC | 54 |
| AQ | GUGUACGCCAAGUCGAGGCCCGACCGUACCCAUACGCGAC | 55 |
| AR | UUAGCUCUACUUUCCUCUUCAGUAAGACUAACCGCUUCUU | 56 |
| AS | UCCAAGCGGAGGCCCCGCACCCACCCUCCAACGGGCACGG | 57 |
| AT | UAUCGCUCCACAACGACUCCCGUGGACUACCCAAUUCCAA | 58 |
| AU | GUCGUGCCCAAGUGAAGGCCUCACGCACGCAUCCUAACCU | 59 |
| AV | AAGAUCUGCGCCAGCACAAUCACCAUCGUCCUGAGAAUGG | 60 |
| AW | AUGCCAAGCAGUGGCCCUGCCACCCACCUAUCACUGUCGA | 61 |
| AX | AACAGACCAAGCAGCGGCCCUGCUCUGCCAUCAUACGCCU | 62 |
| AY | GUCAUUCGCUGACGAAUCAACAUGAAUUCCUAACUGCUGA | 63 |
| AZ | ACACGCCAAGCUGGUAGCCCCAGCCGUGCCCAUUACGGCC | 64 |
| BA | UAGCCAAGCAGCAGCCCUGCCAACCCAUCCUACCCGGGCG | 65 |
| BB | GCCCAAGGCGAGGCCCGCCGCUCCAUCCAGACGCUGAGGG | 66 |
| BC | AAGAUCUCGUCAUGCUUUGACGUCAAUCACCAUUGUUCCC | 67 |
| BD | AUCCCCCAGGAUGAGCACGUUGCCAUGGACUGGCUAUCC | 68 |
| BE | CUGUUACAGUCUCGCGUAACCCCCCCAUCGAUGUCCUCGA | 69 |
| BF | AGCCAGCUUUCGGCAAACCGAAUUCACUCCACCCUGCUCA | 70 |
| BG | CACGGUAUAACCUCCUCAUAUACCUGCUGUGCCACCCGCG | 71 |
| BH | CCGGAAGAUCUGCUCGCACUAGCCGGAGCCCAAUCACGGC | 72 |
| BI | CCUGCCGAACGGCUAAGUCGCAGCCCGACCCGCGGCAGGG | 73 |
| BJ | CUCCGACCCGCGGACGAAGUCAACUUCCACAGUCCCACAC | 74 |
| BK | ACAUUAGGAUCUGCGUGAUGGGGAUCACCCGCUACAUGUC | 75 |
| BL | UCUAAGAUGGGGAAGAUCUCCGGAGCACCGGGCAAUCACC | 76 |
| BM | CUAUUCGAGUUCCCACGAAUCCCCCAUCGAGAACCUAC | 77 |
| BN | UGCCAAGCCGAGGCCCGGCCAGCAUCCCUCACGAGAGAGG | 78 |
| BO | GCCAAGCACGUAGCCCGUGCCCCCACCCGCCUGUGUGCUG | 79 |
| BP | UGCCAAGCACGAAGCCCGUGCCCCCAUCCAGAGUGUGAGA | 80 |
| BQ | AGCCAGCUUUUGCAUACCACGUGCAAUUCACUCCACCCGUCA | 81 |
| BR | CUUUGUAAACCCGGCAAACAAAAUCAACUUCCAUCAUCAA | 82 |
| BS | CCAUUGUAGCGACCACACAAUUCCCCAUCGGACAGCAUGG | 83 |
| BT | CUCUCGCCGUUCCCAGGCACGACAAAAUCAACUUCCCGCU | 84 |
| BU | AAGCCAAGCCGCGGCCCGGCCUUCCCAUGUGCUACUAGAG | 85 |
| BV | CCAAAUGCCAAAGCCGUAGCCCGGCCAGUAGCCCACACGUC | 86 |
| BW | CCAUUACGCGACGUAAUUCCCCCAUCGUUUCCUCGUUAAG | 87 |
| BX | CCAUCUAGAUCUCCGUAGAUUCCCCGGCUCUUUCUCGC | 88 |
| BY | ACUGUCUGCAUACACGGUAUGCCCAACGCCAUCCAAACCG | 89 |
| BZ | ACCUGCGGCUAUUGCCAGCGCCAUAAGACCCUCCACAGUA | 90 |

TABLE 4
Variant Nucleolin Aptamer Sequences without 5′ and 3′ Constant
Regions from All Families

| Family | | SEQ ID |
|---|---|---|
| Name | All Family Sequences | NO: |
| A | CCAUCUAGAUCUCCGUAGAUUCCCCCGGCUCUUUCUCGC | 91 |
| | CCAUCUAGAUCUCCGUAGAUUCCCCCGGCUCUUCCUCGC | 92 |
| | CCAUCUAGAUCUCCGUAGAUUCCCCCAGCUCUUUCUCGC | 93 |
| B | AGCCAGCUUUGCAUACCACGUGCAAUUCACUCCACCCGUCA | 94 |
| | AGCCAGCUUUGCAUACCACGUGCAAUUCACUCCACCCGUCG | 95 |
| C | AAGAUCUGCUAAGUGCACGCACAAUCACCAUCGAGCGUCU | 96 |
| | AAGAUCUGCUAAGUGCACGCACAAUCACCAUCGAGCGUCC | 97 |
| | AAGAUCUGCUAAGUGCACGCACAAUCACCAUCGAGCGCCU | 98 |
| | AAGAUCUGCUAAGUGCACGCACAAUCACCAUCGAGCGUC | 99 |
| | AAGAUCUGCUAAGUGCACGCACAAUCACCAUCGAGCGACU | 100 |
| D | CACAUGGUACGCCCAAAGCGAGGCCCGCUGCGUAGUGC | 101 |
| | CACACGGUACGCCCAAAGCGAGGCCCGCUGCGUAGUGC | 102 |
| E | CACGGUCCAGCGCUAACUGUACCUGCUGUGCCACCCACCG | 103 |
| | CACGGUCCAGCGCUAACUGUACCUGCUGUGCCACCCACCA | 104 |
| | CACGGUCCAGCGCUAACUGUACCUGCUGUGCCACCCACUG | 105 |
| | CACGGUCCAGCGCUAACUGUACCUGCUGUGCCACCCACCU | 106 |
| | CACGGUCCAGCGCUAACUGUACCUGCUGUGCCACCCGCCG | 107 |
| F | ACCACGCGCCAACGUGUCAGCUACACGCCGUGUUCCCCGG | 108 |
| | ACCACGCGCCAACGUGUCAGCUACACGCCGUGUUCCCCGA | 109 |
| | ACCACGCGCCAACGUGUCAGCUACACGCCGUGUUCCCCG | 110 |
| | CCACGCGCCAACGUGUCAGCUACACGCCGUGUUCCCCGG | 111 |
| G | AAGAUCCUCGCGCAUCUGCCGAGCAAUCACCAUCGGACG | 112 |
| | AAGAUCCUCGCGCAUCUGCCGAGCAAUCACCAUCGGACC | 113 |
| | AAGAUCCUCGCGCAUCUGCCGAGCAAUCACCAUCGGACA | 114 |
| | AAGAUCCUCGCGCAUCUGCCGAGCAAUCACCAUCGGACU | 115 |
| | AAAGAUCCUCGCGCAUCUGCCGAGCAAUCACCAUCGGACG | 116 |
| | AAGAUCCUCGCGCACCUGCCGAGCAAUCACCAUCGGACG | 117 |
| H | CCAAAUGCCAAGCCGUAGCCCGGCCAGUAGCCCACACGUC | 118 |
| | CCAAAAUGCCAAGCCGUAGCCCGGCCAGUAGCCCACACGUC | 119 |
| | CCAAAUGCCAAGCCGUAGCCCGGCCAGUAGCCCACACGAC | 120 |
| | CCAAAUGCCAAGCCGUAGCCCGGCCAGUAGCCCACACGUA | 121 |
| I | UGCCAAGCCGAGGCCCGGCCACCAUCCACUGAUAGUGGGC | 122 |
| | UGCCAAGCCGAGGCCCGGCCACCAUCCACUGAUAGUGGGA | 123 |
| | UGCCAAGCCGAGGCCCGGCCACCAUCCACUGAUAGUGGG | 124 |
| | UGCCAAGCCGAGGCCCGGCCACCAUCCACUGAUAGUGGGU | 125 |
| J | AAGAUCCUGACGCGACACAGCAAUCACCAUCGAACCAGCU | 126 |
| | AAGAUCCUGACGCGACACAGCAAUCACCAUCGAACCAGCC | 127 |
| K | AAGAUCUGCGGCAACGCACAAUCACCAUCGAUUCCGAAUU | 128 |
| | AAGAUCUGCGGCAACGCACAAUCACCAUCGAUUCCGAAUG | 129 |
| | AAGAUCUGCGGCAACGCACAAUCACCAUCGAUUCCGAAUC | 130 |
| | AAGAUCUGCGGCAACGCACAAUCACCAUCGAUUCCGAACU | 131 |
| | AAGAUCUGCGGCAACGUACAAUCACCAUCGAUUCCGAAUU | 132 |
| L | GAGCUCUCGAUUUCCUCCGCGACACCCAUCCAAACCUCA | 133 |
| | AGCUCUCGAUUUCCUCCGCGACACCCAUCCAAACCUCA | 134 |
| | GAGCUCUCGAUUUCCUCCGCGACACCCAUCCAAACCUCG | 135 |
| M | CUCUCCGGUCUACCAUCCGGACCGGCGACAAAGUCAACUU | 136 |
| | CUCUCCGGUCUACCACCCGGACCGGCGACAAAGUCAACUU | 137 |
| N | AAGAUCUGCUAUGCACAAUCACCAUCGGGCGCUCCGGGGAA | 138 |
| | AAGAUCUGCUAUGCACAAUCACCAUCGGGCGCUCCGGGAA | 139 |
| | AAGAUCUGCUACGCACAAUCACCAUCGGGCGCUCCGGGGAA | 140 |
| O | UUGACUCUGCUGCGUAGUUCGCACCAAGAUCAACCACUUC | 141 |
| | UUGACUCUGCUGCGUAGUUCGCACCAAGAUCAACCACUUCC | 142 |
| | UUGACUCUGCUGCGUAGCUCGCACCAAGAUCAACCACUUC | 143 |
| | UUGACUCUGCUGCGCAGUUCGCACCAAGAUCAACCACUUC | 144 |
| | UUGACUCUGCUGCGUAGUCCGCACCAAGAUCAACCACUUC | 145 |
| P | UACCAAGUCGUGGCCCGACUACCCAGCACGAUGCGCAA | 146 |
| | UACCAAAGUCGUGGCCCGACUACCCAGCACGAUGCGCAA | 147 |
| | UACCAAGUCGUGGCCCGACUACCCAGCACGGUGCGCAA | 148 |
| | UACCAAGUCGUGGCCCGACUACCCAGCACGAUGCGCAG | 149 |
| | UACCAAGUCGUGGCCCGACUACCCAGCACAAUGCGCAA | 150 |
| | UACCAAGUCGCGGCCCGACUACCCAGCACGAUGCGCAA | 151 |
| Q | CUAUUCGAGUUCCCACGAAUCCCCCCAUCGAGAACCUAC | 152 |
| | CUAUUCGAGUUCCCACGAAUCCCCCCAUCGAGAACCUA | 153 |
| | CUAUUCGAGUUCCCACGAAUCCCCCCAUCGAGAACCUAU | 154 |
| | CUAUUCGAGUUCCCACGAAUCCCCCCAUCGAGAACCUAA | 155 |
| R | UGCCAAGCCGAGGCCCGGCCACCGUCCCCGCGGCUGAUGA | 156 |
| | UGCCAAAGCCGAGGCCCGGCCACCGUCCCCGCGGCUGAUGA | 157 |
| | UGCCAAGCCGAGGCCCGGCCACCGUCCCCGCGGCUGAUCGA | 158 |
| | UGCCAAGCCGAGGCCCGGCCACCGUCCCCGCGGCUGAUGG | 159 |
| | UGCCAAGCCGAGGCCCGGCCACCGUCCCCGCGGCUGACGA | 160 |
| S | AAUGAUCUCGCCAAUGGGCGACAAUCACCAUGUCUUCACA | 161 |
| | AACGAUCUCGCCAAUGGGCGACAAUCACCAUGUCUUCACA | 162 |
| | AAUGAUCUCGCCAAUGGGCGACAAUCACCAUGUCUUCACG | 163 |
| | AAUGAUCUCGCCAAUGUGCGACAAUCACCAUGUCUUCACA | 164 |
| T | UCAGUGCGCCAAGUGGAGGCCCCACCGCAGCCCAUCAA | 165 |
| | UCAGUGCGCCAAGUGGAGGCCCCACCGCAGCCCAUCGA | 166 |
| | UCAGUGCGCCAAGUGGAGGCCCCACCGCAGCCCAUCAG | 167 |
| U | UGUAUGCCAGCUUUGACGAUAACUGUCGCGCGUCAAUUCA | 168 |
| V | UACGCCAAAGUGGAGCCCACUCGUACCCCAUCAUGAGCUG | 169 |
| | UACGCCAAAGUGGAGCCCACUCGUACCCCAUCAUGAGCCUG | 170 |
| | UACGCCAAAGUGGAGCCCACUCGUACCCCAUCAUGAGCUC | 171 |
| | UACGCCAAAGUGGAGCCCACUCGUACCCCAUCAUGGGCUG | 172 |
| | UACGCCAAAGUGGAGCCCACUCGUAUCCCAUCAUGAGCUG | 173 |
| | UACGCCAAAGUGGAGCCCACUCGUACCCCAUCGUGAGCUG | 174 |
| | UACGCCAAAGUGGAGCCCACUCGUACUCCAUCAUGAGCUG | 175 |
| | CACGCCAAAGUGGAGCCCACUCGUACCCCAUCAUGAGCUG | 176 |
| | UACGCCAAAGUGGAGCCCACUCGCACCCCAUCAUGAGCUG | 177 |
| | UACGCCAAAGUGGAGCCCACUCGUACCCCAUCAUGAGCUA | 178 |
| W | CCGCCAGCUUUGGGUACCCUGACCAAUUCACGGCCAUCCA | 179 |
| | CCGCCAGCUUUGGGUACCCUGACCAAUUCACGGCCAUCCG | 180 |
| | CCGCCCAGCUUUGGGUACCCUGACCAAUUCACGGCCAUCCA | 181 |
| X | GUAAUUGUCUGAGACCACCGGACAAUCAACAAGAAAUCCU | 182 |
| | GUAAUUGUCUGAGACCACCGGACAAUCAACAAGAAAAUCCU | 183 |
| | UAAUUGUCUGAGACCACCGGACAAUCAACAAGAAAUCCU | 184 |
| Y | UCAGGCCAAAGUGUGAUAGCCACACCCGCACCCAUCAGGA | 185 |
| | UCAGGCCAAAGUGUGAUAGCCACACCCGCACCCAUCAGA | 186 |
| | UCAGGCCAAAGUGUGAUAGCCACACCCGCACCCAUCAGG | 187 |
| Z | CCGACCGCCGACCAGGGUGCCACUCGUACCCCUGUCCGCC | 188 |
| | CCGACCGCCGACCAGGGUGCCACUCGUACCCCUGUCCGCCC | 189 |
| | CCGACCGCCGACCAGGGUGCCACUCGUACCCCUGUCCCGCC | 190 |
| | CCGACCGCCGACCAGGGUGCCACUCGUACCCCUGUCCGC | 191 |
| AA | UGCCAAGUCGAAGCCCGACCACGCCAUCCCUAACAGUGCC | 192 |
| | UGCCAAAGUCGAAGCCCGACCACGCCAUCCCUAACAGUGCC | 193 |
| | UGCCAAGUCGAAGCCCGACCACGCCAUCCCUAACAGUGC | 194 |
| | UGCCAAGUCGAAGCCCGACCACGCCAUCCCUAACGGUGCC | 195 |
| | UGCCAAGUCGAAGCCCGACCACGCCAUCCCUAACAGUGCA | 196 |
| | UGCCAAGUCGAGGCCCGACCACGCCAUCCCUAACAGUGCC | 197 |
| | UGCCAAGCCGAAGCCCGACCACGCCAUCCCUAACAGUGCC | 198 |
| AB | ACUUGUGCUGAGUCGCCAAAGUGAGGCCCACUCGCCAGCA | 199 |
| | GCUUGUGCUGAGUCGCCAAAGUGAGGCCCACUCGCCAGCA | 200 |
| | ACCUGUGCUGAGUCGCCAAAGUGAGGCCCACUCGCCAGCA | 201 |
| AC | CCGCCAGCUCCUCUGAGGCACAAGAGGUUCACGGUGAUCC | 202 |
| | CCGCCAGCUCCUCUGAGGCACAAGAGGUUCACGGUGAUCCC | 203 |
| AD | CACCAGGUUCUGCUGUCCCCAAGCGCUGACCCAUCCUUCC | 204 |
| | CACCAGGUUCUGCUAUCCCCAAGCGCUGACCCAUCCUUCC | 205 |
| | CACCAGGUUCUGCUGUCUCCAAGCGCUGACCCAUCCUUCC | 206 |
| | CACCAGGUUCUGCUGUUCCCAAGCGCUGACCCAUCCUUCC | 207 |
| | CACCAGGUCCUGCUGUCCCCAAGCGCUGACCCAUCCUUCC | 208 |
| | CACCAGGCUCUGCUGUCCCCAAGCGCUGACCCAUCCUUCC | 209 |
| | CACCAGGUUCUGCUGUCCUCAAGCGCUGACCCAUCCUUCC | 210 |
| AE | AAGAUCCGGUAACUCCCCACCGCAAUCACCGUCGACUACU | 211 |
| | AAGAUCCGGUGACUCCCCACCGCAAUCACCGUCGACUACU | 212 |
| | AAGAUCCGGUAACUCCCUACCGCAAUCACCGUCGACUACU | 213 |
| | AAAGAUCCGGUAACUCCCCACCGCAAUCACCGUCGACUACU | 214 |
| AF | CCAUCUAGAUCUCCGUAGAUUCCCCCCGGCUCUUUCUCGC | 215 |
| | CCAUCUAGAUCUCCGUAGAUUCCCCCGGGCUCUUUCUCGU | 216 |
| | CCAUCUAGAUCUCCGUAGAUUCCCCCGGGCUCUUUCUCGA | 217 |
| | CCAUCUAGAUCUCCGUAGAUUCCCCCGGGCUCUUUCUCG | 218 |
| | CCAUCUAGAUCUCCGUAGAUUCCCCCGGGCUCUUUCUCGC | 219 |
| | CCAUCUAGAUCUCCGUAGAUUCCCCCGGGCUCUUUCUCAC | 220 |
| | CCAUCUAGAUCUCCGUAGAUUUCCCCCGGCUCUUUCUCGC | 221 |
| | CCAUCUAGAUCUCCGUAGAUUCCCCCGGGCUCUUCCUCGC | 222 |
| | CCAUCUAGAUCUCCGUAGAUUCCCCCGGGCUCUCUCUCGC | 223 |
| | CCAUCUAGAUCUCCGUAGAUUCCCCCGGGCUCUUUCUUGC | 224 |
| | CCAUCUAGAUCUCCGUAGAUUCCCCCGGGCCCUUUCUCGC | 225 |
| | CCAUCUAGAUCUCCGUAGAUUCCCCCGGGCUCUUUCUCUC | 226 |
| | CCAUCUAGAUCUCCGUAGAUUCCCCCGGCCUCUUUCUCGC | 227 |
| | CCAUCUAGAUCUCCGUAGAUUCCCCCGGGCUCUUUCUCCC | 228 |
| | CCAUCUAGAUCUCCGUAGAUUCCCCCGGGCUCUUUCUCGUC | 229 |
| AG | CCAUCUGAACCCACAGAUUCCCCCAUCAUCAGCCACAGUG | 230 |
| | CCAUCUGAACCCACAGAUUCCCCCAUCAUCAGCCACAGUA | 231 |
| | CCAUCUGAACCCACAGAUUCCCCCAUCAUCAGCCACAGCG | 232 |
| | CCAUCUGAACCCACAGAUUCCCCCAUCAUCAGCCACAGUC | 233 |
| | CCAUCUGAACCCACAGAUUCCCCCAUCAUCAGCCACGGUG | 234 |
| AH | CACUAAGUUGGUAGCCCCAACUGCCCCGACACGAGGAUGU | 235 |
| | CACUAAGUUGGUAGCCCCAACUGCCCCGACACGAGGAUGUC | 236 |
| | CACUAAGUUGGUAGCCCCAACUGCCCCGACACGAGGAUGC | 237 |
| AI | UUGUGCUCCGUGGCUCCCCGGACCAACCGCUUCCAGCAGU | 238 |
| | UUGUGUUCCGUGGCUCCCCGGACCAACCGCUUCCAGCAGU | 239 |
| | UUGUGCUCCGUGGCUCCCCGGACCAACCGCUUCCAGCAGC | 240 |
| | UUGCGCUCCGUGGCUCCCCGGACCAACCGCUUCCAGCAGU | 241 |
| AJ | CAAUCACGCGUAGUACGUCGCGGAAGAUCCCCAUGCCGA | 242 |
| | CAAUCACGCGUAGUACGUCGCGGAAGAUCCCCAUGCCGG | 243 |
| | CAAUCACGCGUAGUACGUCGCGGAAGAUCCCCAUGCCAA | 244 |
| | CAAUCACGCGUAGUACGUCGCGGAAGAUCCCCAUGCCGU | 245 |
| | CAAUCACGCGUAGCACGUCGCGGAAGAUCCCCAUGCCGA | 246 |
| | CAAUCACGCGUAGUACGUCGCGGAGGAUCCCCAUGCCGA | 247 |
| AK | CACAUGGUACGCCCAAAAGCGAGGCCCGCUGCGUAGUGC | 248 |
| | CACAUGGUACGCCCCAAAGCGAGGCCCGCUGCGUAGUGC | 249 |
| | CACAUGGUACGCCCAAAGCCGAGGCCCGCUGCGUAGUGC | 250 |
| | CACAUGGUACGCCCAAAAGCGAGGCCCGCUGCGUAGUG | 251 |
| AL | UGCCAUACGCGGUUCGAAGUCGAAGCCCGACAACCCGGCA | 252 |
| | UGCCAUACGCGGUUCGAAGUCGAAGCCCGACAACCCCGGCA | 253 |
| | UGCCAUACGCGGUUCGAAGUCGAGGCCCGACAACCCGGCA | 254 |
| AM | GUUAUUCACAUGCCUCCCGUGAAUCAACAAGAAUUCCUUG | 255 |
| | UUAUUCACAUGCCUCCCGUGAAUCAACAAGAAUUCCUUG | 256 |
| | GUUAUUCACAUGCCUCCCGUGAAUCAACAAGAAUUCCUCG | 257 |
| | GUUAUUCACAUGCCUCUCGUGAAUCAACAAGAAUUCCUUG | 258 |
| | | 259 |
| AN | AAAGAUCUAGACUGUAAGUCUCCAAUCGCCCAGUUAAUUC | 260 |
| | AAAAGAUCUAGACUGUAAGUCUCCAAUCGCCCAGUUAAUUC | 261 |
| | AAAGAUCUAGACUGUAAGUCUCCAAUCGCCCAGUAAUUC | 262 |
| AO | GCCCAAUCGCCAGUGGAACGCGCUGAAGGAUCUGCACCC | 263 |
| | GCCCAAUCGCCAGUGGAACGCGCUGAAGGAUCUGCACC | 264 |
| | GCCCAAUCGCCAGUGGAACGCACUGAAGGAUCUGCACCC | 265 |
| | GCCCAAUCGCCAGUGGAACGCGCUGAAGGAUCUGCACCCC | 266 |
| | CCCAAUCGCCAGUGGAACGCGCUGAAGGAUCUGCACCC | 267 |
| | GCCCAAUCGCCAGCGGAACGCGCUGAAGGAUCUGCACCC | 268 |
| AP | UGCAACGUAAAAGAGAGUCAUCUCAGGCUAGUCGUCUACC | 269 |
| | UGCAACGUAAAAGAGAGUCAUCUCAGGCUAGUCGUCUAC | 270 |
| AQ | GUGUACGCCAAGUCGAGGCCCGACCGUACCCAUACGCGAC | 271 |
| | UGUACGCCAAGUCGAGGCCCGACCGUACCCAUACGCGAC | 272 |
| | GUGUACGCCAAGUCGAGGCCCGACCGUACCCAUACGCGGC | 273 |
| | GUGUACGCCAAGUCGAGGCCCGACCGUACCCAUACGCGAU | 274 |
| AR | UUAGCUCUACUUUCCUCUUCAGUAAGACUAACCGCUUCUU | 275 |
| | UUAGCUCUACUUUCCUCUUCAGUAAGACUAACCGCUUCCU | 276 |
| | UUAGCUCUACUUUCCUCUUCAGUAAGACUAACCGCUUCUC | 277 |
| | UUAGCUCUACUUUCCUCUUCAGUAAGACUAACCGCUCCUU | 278 |
| AS | UCCAAGCGGAGGCCCCGCACCCACCCUCCAACGGGCACGG | 279 |
| | UCCAAGCGGAGGCCCCGCACCCACCCUCCAACGGGCACGC | 280 |
| | UCCAAGCGGAGGCCCCGUACCCACCCUCCAACGGGCACGG | 281 |
| | UCCAAGCGGAGGCCCCGCACCCACCCCCCAACGGGCACGG | 282 |
| | UCCAAGCGGAGGCCCCGCACCCACCCUCCAACGGGCACGA | 283 |
| | UCCAAAGCGGAGGCCCCGCACCCACCCUCCAACGGGCACGG | 284 |
| | UCCAAGCGGAGGCCCCGCACCCACCCUCCAACGGGCACAG | 285 |
| AT | UAUCGCUCCACAACGACUCCCGUGGACUACCCAAUUCCAA | 286 |
| | UAUCGCUCCACAACGACUCCCGUGGACUACCCAAUUCCAG | 287 |
| | UAUCGCUCCACAACGACUCCCGUGGACUACCCAAUUCCAAA | 288 |
| | UAUCGCUCCACAACGACUCCCGUGGACUACCCAAUUCCAU | 289 |
| AU | GUCGUGCCCAAGUGAAGGCCUCACGCACGCAUCCUAACCU | 290 |
| | UCGUGCCCAAGUGAAGGCCUCACGCACGCAUCCUAACCU | 291 |
| | GUCGUGCCCAAGUGAAGGCCUCACGCACGCAUCCUAACCC | 292 |
| AV | AAGAUCUGCGCCAGCACAAUCACCAUCGUCCUGAGAAUGG | 293 |
| | AAGAUCUGCGCCAGCACAAUCACCAUCGUCCUGAGAAUGC | 294 |
| | AAGAUCUGCGCCAGCACAAUCACCAUCGUCCUGAGAAUGA | 295 |
| | AAGAUCUGCGCCAGCACAAUCACCAUCGUCCUGAGAGUGG | 296 |
| | AAGAUCUGCGCCAGCACAAUCACCAUCGUCCUGGGAAUGG | 297 |
| AW | AUGCCAAGCAGUGGCCCUGCCACCCACCUAUCACUGUCGA | 298 |
| | AUGCCAAGCAGUCGGCCUGCCACCCACCUAUCACUGUCGA | 299 |
| | AUGCCAAGCAGUGGCCCUGCCACCCACCUAUCACUAUCGA | 300 |
| | AUGCCAAGCAGUGGCCCUGCCACCCACCUACCACUGUCGA | 301 |
| | AUGCCAAGCAGCGGCCCUGCCACCCACCUAUCACUGUCGA | 302 |
| AX | AACAGACCAAGCAGCGGCCCUGCUCUGCCAUCAUACGCCU | 303 |
| | GACAGACCAAGCAGCGGCCCUGCUCUGCCAUCAUACGCCU | 304 |
| | AACAGACCAAGCAGUGGCCCUGCUCUGCCAUCAUACGCCU | 305 |
| | AACAGACCAAGCAGCGGCCCUGCUCUGCCAUCAUACGCCC | 306 |
| | AACAGACCAAGCAGCGGCCCUGCUCUGCCAUCAUACACCU | 307 |
| | ACAGACCAAGCAGCGGCCCUGCUCUGCCAUCAUACGCCU | 308 |
| | AACAGACCAAGCAGCGGCCCUGCUCUGCCAUCAUACGCCCU | 309 |
| AY | GUCAUUCGCUGACGAAUCAACAUGAAUUCCUAACUGCUGA | 310 |
| | UCAUUCGCUGACGAAUCAACAUGAAUUCCUAACUGCUGA | 311 |
| | GUCAUUCGCUGACGAAUCAACAUGAAUUCCUAACUGCCGA | 312 |
| | GUCAUUCGCUGACGAAUCAACAUGAAUUCCUAACUGCUGG | 313 |
| AZ | ACACGCCAAGCUGGUAGCCCCAGCCGUGCCCAUUACGGCC | 314 |
| | ACACGCCAAGCUGGUAGCCCCAGCCGUGCCCAUUACGGC | 315 |
| | ACACGCCAAGCUGGUAGCCCCAGCCGUGCCCAUUACGGUC | 316 |
| | ACACGCCAAGCUGGUAGCCCCAGCCGUACCCAUUACGGCC | 317 |
| BA | UAGCCAAGCAGCAGCCCUGCCAACCCAUCCUACCCGGGCG | 318 |
| | UAGCCAAGCAGCAGCCCUGCCAACCCAUCCUACCCGGCG | 319 |
| | UAGCCAAGCAGCAGCCCUGCCAACCCAUCCUACCCGGGCA | 320 |
| | UAGCCAAGCAGCAGCCCUGCCAACCCAUCCUACCCGGGUG | 321 |
| | UAGCCAAGCAGCGGCCCUGCCAACCCAUCCUACCCGGGCG | 322 |
| BB | GCCCAAGGCGAGGCCCGCCGCUCCAUCCAGACGCUGAGGG | 323 |
| | GCCCAAGGCGAGGCCCGCCGCUCCAUCCAGACGCUGAGG | 324 |
| | CCCAAGGCGAGGCCCGCCGCUCCAUCCAGACGCUGAGGG | 325 |
| | CCCAAGGCGAGGCCCGCCGCUCCAUCCAGACGCUGAGG | 326 |
| | GCCCAAGGCGAGGCCCGCCGCUCCAUCCAGACGCUGAGGC | 327 |
| | GCCCAAAGGCGAGGCCCGCCGCUCCAUCCAGACGCUGAGGG | 328 |
| | GCCCAAGGCGAGGCCCGCCGCUCCAUCCAGACGCUGAGGA | 329 |
| | GCCCCAAGGCGAGGCCCGCCGCUCCAUCCAGACGCUGAGGG | 330 |
| BC | AAGAUCUCGUCAUGCUUUGACGUCAAUCACCAUUGUUCCC | 331 |
| | AAGAUCUCGUCAUGCUUUGACGUCAAUCACCAUUGUUCC | 332 |
| | AAGAUCUCGUCAUGCUUUGACGCCAAUCACCAUUGUUCCC | 333 |
| | AAGAUCUCGUCAUGCUUUGACGUCAAUCACCAUUGUUCCA | 334 |
| | AAGAUCUCGUCAUGCUUUGACGUCAAUCACCAUUGUUCCU | 335 |
| | AAGAUCUCGUCAUGCUUUGACGUCAAUCACCAUUGUUCCCC | 336 |
| | AAAGAUCUCGUCAUGCUUUGACGUCAAUCACCAUUGUUCCC | 337 |
| | AAGAUCUCGUCAUGCCUUGACGUCAAUCACCAUUGUUCCC | 338 |
| BD | AUCCCCCAGGAUGAGCACGUUGCCAUGGACUGGCUAUCC | 339 |
| | AUCCCCAGGAUGAGCACGUUGCCAUGGACUGGCUAUCC | 340 |
| BE | CUGUUACAGUCUCGCGUAACCCCCCCAUCGAUGUCCUCGA | 341 |
| | CUGUUACAGUCUCGCGUAACCCCCCCAUCGAUGUCCUCGG | 342 |
| | CUGUUACAGUCUCGAGUAACCCCCCCAUCGAUGUCCUCGA | 343 |
| | CUGUUACAGUCUCGCGUAACCCCUCCAUCGAUGUCCUCGA | 344 |
| | CUGUUACAGCCUCGCGUAACCCCCCCAUCGAUGUCCUCGA | 345 |
| | CUGUUACAGUCUCCCGUAACCCCCCCAUCGAUGUCCUCGA | 346 |
| BF | AGCCAGCUUUCGGCAAACCGAAUUCACUCCACCCUGCUCA | 347 |
| | AGCCAGCUUUCGGCAAACCGAAUUCACUCCACCCUCCUCA | 348 |
| | AGCCAGCUUUCGGCAAACCGAAUUCACUCCGCCCUGCUCA | 349 |
| | AGCCAGCUUUCGGCAAACCGAAUUCACUCCACCCUGCU | 350 |
| | AGCCAGCUUUCGGCGAACCGAAUUCACUCCACCCUGCUCA | 351 |
| | AGCCAGCUUUCGGCAAACCGAAUUCACUCCACCCUGCUCG | 352 |
| | AGCCAGCUUUCGGCAAACCGAAUUCACUCCACCCUGCUC | 353 |
| | AGCCAGCUUUCGGCAAACCGAAUUCACUCCACCCUGCACA | 354 |
| BG | CACGGUAUAACCUCCUCAUAUACCUGCUGUGCCACCCGCG | 355 |
| | CACGGUAUAACCUCCUCAUAUACCUGCUGUGCCACCCGCA | 356 |
| | CACGGUAUAACCUCCUCAUAUACCUGCUGUGCCACCCACCG | 357 |
| | CACGGUAUAACCUCCUCAUAUACCUGCUGUGCCACCCGCU | 358 |
| | CACGGUAUAACCUCCUCAUAUACCUGCUGUGCCACCCACG | 359 |
| | CACGGUAUAACCUCCUCAUAUACCUGCUGUGCCACCCGUG | 360 |
| | CACGGUAUAACCUCCUCAUAUACCUGCUGUGCCGCCCGCG | 361 |
| BH | CCGGAAGAUCUGCUCGCACUAGCCGGAGCCCAAUCACGGC | 362 |
| | CCGGAAGAUCUGCUCGCACUAGUCGGAGCCCAAUCACGGC | 363 |
| | CCGGAGGAUCUGCUCGCACUAGCCGGAGCCCAAUCACGGC | 364 |
| | CCGGAAGAUCUGCUCGCAUUAGCCGGAGCCCAAUCACGGC | 365 |
| BI | CCUGCCGAACGGCUAAGUCGCAGCCCGACCCGCGGCAGGG | 366 |
| | CCUGCCGAACGGCUAAGUCGCAGCCCGACCCGCGGCAGG | 367 |
| | CCUGCCGAACGGCUAAGUCGCAGCCCGACCCGCGGCAGGA | 368 |
| | CCUGCCGAACGGCCAAGUCGCAGCCCGACCCGCGGCAGGG | 369 |
| | CCUGCCGAACGGCUAAGUCGCGGCCCGACCCGCGGCAGGG | 370 |
| BJ | CUCCGACCCGCGGACGAAGUCAACUUCCACAGUCCCACAC | 371 |
| | CUCCGACCCGCGGACGAAGUCAACUUCCACAGUCCCACAA | 372 |
| | CUCCGACCCGCGGACGAAGUCAACUUCCACAGUCCCACACAC | 373 |
| | CUCCGACCCGCGGACGAAGUCAACUUCCACAGUCUCACAC | 374 |
| | CUCCGACCCGCGGACGAAGUCAACUUCCACAGUCCCACAU | 375 |
| | CUCCGACCCGCGGACGAAGUCAACUUCCACAGUCCCGCAC | 376 |
| | CUCCGACCCGCGGACGAAGUCAACUUCCACGGUCCCACAC | 377 |
| | CUCCGACCCGCGGACGAAGUCAACUUCCACAGUCCCAUAC | 378 |
| BK | ACAUUAGGAUCUGCGUGAUGGGGAUCACCCGCUACAUGUC | 379 |
| | ACAUUUAGGAUCUGCGUGAUGGGGAUCACCCGCUACAUGUC | 380 |
| | GCAUUAGGAUCUGCGUGAUGGGGAUCACCCGCUACAUGUC | 381 |
| | ACAUUAGGAUCUGCGCGAUGGGGAUCACCCGCUACAUGUC | 382 |
| BL | UCUAAGAUGGGGAAGAUCUCCGGAGCACCGGGCAAUCACC | 383 |
| | UCUAAGAUGGGGAAGAUCUCCGGAGCACCGGGCAAUCACCC | 384 |
| | CCUAAGAUGGGGAAGAUCUCCGGAGCACCGGGCAAUCACC | 385 |
| | UCUAAGGUGGGGAAGAUCUCCGGAGCACCGGGCAAUCACC | 386 |
| | UCUAAGAUGGGGAAGAUCUCCGGAGCGCCGGGCAAUCACC | 387 |
| BM | CUAUUCGAGUUCCCACGAAUCCCCCAUCGAGAACCUAC | 388 |
| | CUAUUCGAGUUCCCACGAAUCCCCCCAUCAGAACCUAC | 389 |
| | CUACUCGAGUUCCCACGAAUCCCCCAUCGAGAACCUAC | 390 |
| | CUAUUCGAGUUCCCACGAAUCCCCCAUCAAGAACCUAC | 391 |
| BN | UGCCAAGCCGAGGCCCGGCCAGCAUCCCUCACGAGAGAGG | 392 |
| | UGCCAAAGCCGAGGCCCGGCCAGCAUCCCUCACGAGAGAGG | 393 |
| | UGCCAAGCCGAGGCCCGGCCAGCAUCCCUCACGAGAGAGC | 394 |
| | UGCCAAGCCGAGGCCCGGCCAGCAUCCCUCACGAGAGAG | 395 |
| | UGCCAAGCCGAGGCCCGGCCAGCAUCCCCCACGAGAGAGG | 396 |
| | UGCCAAGCCGAGGCCCGGCCAGCAUCCCUCACGAGAGAGA | 397 |
| | UGCCAAGCCGGGGCCCGGCCAGCAUCCCUCACGAGAGAGG | 398 |
| | UGCCAAGCCGAGGCCCGGCCAGCAUCCCUCACGAGAGGG | 399 |
| BO | GCCAAGCACGUAGCCCGUGCCCCCACCCGCCUGUGUGCUG | 400 |
| | CCAAGCACGUAGCCCGUGCCCCCACCCGCCUGUGUGCUG | 401 |
| | GCCAAGCACGUAGCCCGUGCCCCCACCCGCCUGUGUGCGG | 402 |
| | GCCAAGCACGUAGCCCGUGCCCCCACCCACCUGUGUGCUG | 403 |
| | GCCAAGCACGUAGCCCGUGCCCCCACCCGCCUGUGUGCUC | 404 |
| | GCCAAGCACGUAGCCCGUGCCCCCACCCGCCUGUGUGCCG | 405 |
| | GCCAAAGCACGUAGCCCGUGCCCCCACCCGCCUGUGUGCUG | 406 |
| | GCCAAGCACGUAGCCCGUGCCCCCACCCGCCUGUGUGCUA | 407 |
| BP | UGCCAAGCACGAAGCCCGUGCCCCCAUCCAGAGUGUGAGA | 408 |
| | UGCCAAAGCACGAAGCCCGUGCCCCCAUCCAGAGUGUGAGA | 409 |
| | UGCCAAGCACGAAGCCCGUGCCCCCAUCCAGAGUGUGGGA | 410 |
| | UGCCAAGCACGAGGCCCGUGCCCCCAUCCAGAGUGUGAGA | 411 |
| | UGCCAAGCACGAAGCCCGUGCCCCCAUUCAGAGUGUGAGA | 412 |
| | UGCCAAGCACGAAGCCCGUGCCCCCAUCCAGAGUGCGAGA | 413 |
| | UGCCAAGCACGAAGCCCGUGCCCCCAUCCAGAGCGUGAGA | 414 |
| | UGCCAAGCACGAAGCCCGUGCCCCCAUCCAGAGUGUGAGG | 415 |
| | UGCCAAGCACGAAGCCCGUGCCCCCAUCCAGGGUGUGAGA | 416 |
| BQ | AGCCAGCUUUUGCAUACCACGUGCAAUUCACUCCACCCGUCA | 417 |
| | AGCCAGCUUUGCCAUACCACGUGCAAUUCACUCCACCCGUCA | 418 |
| | AGCCAGCCUUUGCAUACCACGUGCAAUUCACUCCACCCGUCA | 419 |
| | AGCCAGCUUUUGCAUACCACGUGCAAUUCACUCCACCCGUCG | 420 |
| | AGCCAGCUUUUGCACACCACGUGCAAUUCACUCCACCCGUCA | 421 |
| | AGCCAAGCUUUGCAUACCACGUGCAAUUCACUCCACCCGUCA | 422 |
| BR | CUUUGUAAACCCGGCAAACAAAAUCAACUUCCAUCAUCAA | 423 |
| | CUUUGUAAACCCGGCAAACAAAAUCAACUUCCAUCACCAA | 424 |
| BS | CCAUUGUAGCGACCACACAAUUCCCCAUCGGACAGCAUGG | 425 |
| | CCAUUGUAGCGACCACACAAUUCCCCAUCGGACAGCAUG | 426 |
| | CCAUUGUAGCGACCACACAAUUCCCCAUCGGACAGCGUGG | 427 |
| | CCAUUGUAGCGACCACACAAUUCCCCAUCGGACAGCACGG | 428 |
| | CCAUUGUAGCGACCACACAAUUCCCCAUCGGACAGCAUGC | 429 |
| | CCAUUGUAGCGACCACACAAUCCCCCAUCGGACAGCAUGG | 430 |
| | CCAUUGUAGCGACCACACAAUUCCCCAUCGGACAGCAUGU | 431 |
| BT | CUCUCGCCGUUCCCAGGCACGACAAAAUCAACUUCCCGCU | 432 |
| | CUCUCGCCGUUCCCAGGCGCGACAAAAUCAACUUCCCGCU | 433 |
| | CUCUCGCCGUUCCCGGGCACGACAAAAUCAACUUCCCGCU | 434 |
| | CUCUCGCCGUUCCCAGGCACGACAAAAUCAACUUCCCGCA | 435 |
| BU | AAGCCAAGCCGCGGCCCGGCCUUCCCAUGUGCUACUAGAG | 436 |
| | AAAGCCAAGCCGCGGCCCGGCCUUCCCAUGUGCUACUAGAG | 437 |
| | AAGCCAAAGCCGCGGCCCGGCCUUCCCAUGUGCUACUAGAG | 438 |
| | GAGCCAAGCCGCGGCCCGGCCUUCCCAUGUGCUACUAGAG | 439 |
| | AGCCAAGCCGCGGCCCGGCCUUCCCAUGUGCUACUAGAG | 440 |
| | AAGCCAAGCCGUGGCCCGGCCUUCCCAUGUGCUACUAGAG | 441 |
| | UGCCAAGCCGCGGCCCGGCCUUCCCAUGUGCUACUAGAG | 442 |
| | AAGCCAAGCCGAGGCCCGGCCUUCCCAUGUGCUACUAGAG | 443 |
| BV | CCAAAUGCCAAAGCCGUAGCCCGGCCAGUAGCCCACACGUC | 444 |
| | CCAAAAUGCCAAAGCCGUAGCCCGGCCAGUAGCCCACACGUC | 445 |
| | CCAAAUGCCAAGCCCGUAGCCCGGCCAGUAGCCCACACGUC | 446 |
| BW | CCAUUACGCGACGUAAUUCCCCCAUCGUUUCCUCGUUAAG | 447 |
| | CCAUUACGCGACGUAAUUCCCCCAUCGUCUCCUCGUUAAG | 448 |
| | CCAUUACGCGACGUAAUUCCCCCAUCGCUUCCUCGUUAAG | 449 |
| | CCAUUACGCGGCGUAAUUCCCCCAUCGUUUCCUCGUUAAG | 450 |
| | CCAUUACGCGACGUAAUUCCCCCAUCGUUUCCUCGUUAGG | 451 |
| | CCAUUACGCGACGUAAUUCCCCCAUCGUUUCCUCGCUAAG | 452 |
| | CCAUUACGCGACGUAAUUCCCCCAUCGUUUCCUCGUUAUG | 453 |
| | CCAUUACGCGACGUAAUUCCCCCAUCGUUUCCUCGUUAAA | 454 |
| BX | CCAUCUAGAUCUCCGUAGAUUCCCCGGCUCUUUCUCGC | 455 |
| | CCAUCUAGAUCUCCGUAGAUUCCCCAGCUCUUUCUCGC | 456 |
| | CCAUCUAGAUCUCCGUAGAUCCCCCGGCUCUUUCUCGC | 457 |
| | CCAUCUAGAUCUCCGUAGAUUCCCCCGCUCUUUCUCGC | 458 |
| | CCAUCUAGAUCUCCGUAGAUUCCCCGGCUCUUCCUCGC | 459 |
| | CCAUCUAGAUCUCCGUGAUUCCCCCGGCUCUUUCUCGC | 460 |
| | CCAUCUAGAUCUCCGUAGUUCCCCCGGCUCUUUCUCGC | 461 |
| | CCAUCUAGAUCCCCGUAGAUUCCCCGGCUCUUUCUCGC | 462 |
| | CCAUCUAGAUCUCCGUAGAUUCCCCGGCUCCUUCUCGC | 463 |
| | CCAUCUAUAUCUCCGUAGAUUCCCCGGCUCUUUCUCGC | 464 |
| BY | ACUGUCUGCAUACACGGUAUGCCCAACGCCAUCCAAACCG | 465 |
| | ACUGUCUGCAUACACGGUAUGCCCAACGCCAUCCAAACCGC | 466 |
| | ACUGUCUGCAUACAUGGUAUGCCCAACGCCAUCCAAACCG | 467 |
| | ACUGUCUGCAUACACGGUAUGCCCAACGCCAUCCAAAACCG | 468 |
| BZ | ACCUGCGGCUAUUGCCAGCGCCAUAAGACCCUCCACAGUA | 469 |
| | ACCUGCGGCUAUUGCCAGCGCCAUAAGACCCUCCACAGCA | 470 |
| | CCUGCGGCUAUUGCCAGCGCCAUAAGACCCUCCACAGUA | 471 |
| | ACCUGCGGCUAUUGCCAGCGCCAUAAGACCUUCCACAGUA | 472 |
| | ACCUGCGGCUAUUGCCAGCGCCAUAAGACCCUCCGCAGUA | 473 |

TABLE 5
Nucleolin Binding of Aptamer Families A-F

| Clone | Kd (nM) | Bmax (%) | R² |
|---|---|---|---|
| FAM-A | 10.07 | 17.66 | 0.9499 |
| FAM-B | 0.8508 | 25.2 | 0.8335 |
| FAM-C | 0.4285 | 32.76 | 0.869 |
| FAM-D | 0.586 | 53.6 | 0.9447 |
| FAM-E | 1.69 | 23.08 | 0.7941 |
| FAM-F | 0.37 | 33.6 | 0.6520 |

TABLE 6
Nucleolin Aptamer Truncates

| NCL | |
|---|---|
| Aptamer | Sequence |
| Bv1 | GGAAGAGGGAUGGGUGCCAGCUUUGCAUACCACGUGCAAUUCACUCCAC |
| | CCGUCAC (SEQ ID NO: 474) |
| Bv2 | GGGAGAGAGGAAGAGGGAUGGGAGCCAGCUUUGCAUACCACGUGCAAUU |
| | CACUCCACCCGUCAC (SEQ ID NO: 475) |
| Dv1 | GGGAUGGGCACAUGGUACGCCCAAAGCGAGGCCCGCUGCGUAGUGCCAU |
| | AACCCAG (SEQ ID NO: 476) |
| Dv2 | GGGAGAGAGGAAGAGGGAUGGGCACAUGGUACGCCCAAAGCGAGGCCCG |
| | CUGCGUAGUGCC (SEQ ID NO: 477) |
| Ev1 | GGGAUGGGCACGGUCCAGCGCUAACUGUACCUGCUGUGCCACCCACCGCA |
| | UAACCCAGAGGUCGAU (SEQ ID NO: 478) |
| Ev2 | GGGAUGGGCACGGUCCAGCGCUAACUGUACCUGCUGUGCCACCCACCGC |
| | (SEQ ID NO: 479) |
| Ev3 | GGGAGAGAGGAAGAGGGAUGGGCACGGUCCAGCGCUAACUGUACCUGCU |
| | GUGCCACCCACCG (SEQ ID NO: 480) |
| Ev4 | GGGAGAGAGGAAGAGGGAUGGGCACGGUCCAGCGCUAACUGUACC (SEQ |
| | ID NO: 481) |
| Ev5 | GGAAGAGGGAUGGGCACGGUCCAGCGCUAACUGUACCUGCUGUGCCACC |
| | CACC (SEQ ID NO: 482) |
| Fv1 | GGGACCACGCGCCAACGUGUCAGCUACACGCCGUGUUCCCCGG (SEQ ID |
| | NO: 483) |
| Fv2 | GGGACCACGCGCCAACGUGUCAGCUACACGCCGUGUUCCCCGGCAUAACC |
| | CAGAGGUCGAU (SEQ ID NO: 484) |
| Fv3 | GGGAGAGAGGAAGAGGGAUGGGACCACGCGCCAACGUGUCAGCUACACG |
| | CCGUGUUCCCCGG (SEQ ID NO: 485) |

TABLE 7
Ev3 Truncates

| NCL | |
|---|---|
| Aptamer | Sequence |
| Ev3.min21 | GGGAUGGGCACGGUCCAGCGCUAACUGUACCUGCUGUGCCACCC |
| | (SEQ ID NO: 486) |
| Ev3.min22 | GGGAGGAAGAGGGAUGGGCACGGUCCAGCGCUAACUGUACCUGCUG |
| | UGCCACCC (SEQ ID NO: 487) |
| Ev3.min23 | GGGAGGAAGAGGAUGGGCACGGUCCAGCGCUAACUGUACCUGCUGU |
| | GCCACC (SEQ ID NO: 488) |
| Ev3.min24 | GGGAGGAAGAGGGAUGGGCACGGUCCAGCGCACUGUACCUGCUGUG |
| | CCACCC (SEQ ID NO: 489) |
| Ev3.min25 | GGGAGGAAGAGGAUGGGCACGGUCCAGCGCACUGUACCUGCUGUGCC |
| | ACC (SEQ ID NO: 490) |

TABLE 8
Additional Nucleolin Aptamers

| NCL | |
|---|---|
| Aptamer | Sequence |
| Cv1 | GGGAUGGGAAGAUCUGCUAAGUGCACGCACAAUCACCAUCGAGCGUC |
| | UC (SEQ ID NO: 494) |
| Cv2 | GGGAGAGAGGAAGAGGGAUGGGAAGAUCUGCUAAGUGCACGCACAA |
| | UCACCAUCGAGCGUCUC (SEQ ID NO: 495) |
| Ev6 | GGGAGAGAGGAAGAGGGAUGGGCACGGUCCAGCGCUAACUGUACCU |
| | GCUGUGCC (SEQ ID NO: 496) |
| Ev3min2 | GGGAGAGAGAGGGAUGGGCACGGUCCAGCGCUAACUGUACCUGCUG |
| | UGCCACCCACCG (SEQ ID NO: 497) |
| Ev3min3 | GGGAGAGAGGAAGAGGAUGGGCACGGUCCAGCGCUAACUGUACCUG |
| | CUGUGCCACCACCG (SEQ ID NO: 498) |
| Ev3min4 | GGGAGAGAGGAAGAGGGAGGGCACGGUCCAGCGCUAACUGUACCUG |
| | CUGUGCCCCCACCG (SEQ ID NO: 499) |
| Ev3min5 | GGGAGAGAGGAAGAGGGAUGGGUCCAGCGCUAACUGUACCUGCCAC |
| | CCACCG (SEQ ID NO: 500) |
| Ev3min6 | GGGAGAGAGGAAGAGGGAUGGGCGGUCCAGCGCUAACUGUACCUGC |
| | UGCCACCCACCG (SEQ ID NO: 501) |
| Ev3min7 | GGGAGAGAGGAAGAGGGAUGGGCACGGUCCAGCGCUAUGUCUGCUG |
| | UGCCACCCACCG (SEQ ID NO: 502) |
| Ev3min8 | GGGAGAGAGGAAGAGGGAUGGGCACGGUCCAGCGCUAACUGUACCU |
| | GCUGUGCCACCC (SEQ ID NO: 503) |
| Ev3min9 | GGGAGGAAGAGGGAUGGGCACGGUCCAGCGCUAACUGUACCUGCUG |
| | UGCCACCCACCG (SEQ ID NO: 504) |
| Ev3min10 | GGAAGAGGGAUGGGCACGGUCCAGCGCUAACUGUACCUGCUGUGCCA |
| | CCCACCG (SEQ ID NO: 505) |
| Ev3min11 | GAGAGGAAGAGGGAUGGGCACGGUCCAGCGCUAACUGUACCUGCUG |
| | UGCCACCCACCG (SEQ ID NO: 506) |
| Ev3min12 | GGGAGAGAGGAAGAGGGAUGGGCACGGUCCAGCGCUAACUGUACCU |
| | GCUGUGCCACCCAC (SEQ ID NO: 507) |
| Ev3min13 | GGGAGAGAGGAAGAGGGAUGGGCACGGUCCAGCGCUAACUGUACCU |
| | GCUGUGCCACCCCG (SEQ ID NO: 508) |
| Ev3min14 | GGGAGAGAGGAAGAGGGAUGGGCACGGUCCGCGCUAACUGUACCUG |
| | CUGGCCACCCACCG (SEQ ID NO: 509) |
| Ev3min15 | GGGAGAGAGGAAGAGGGAUGGGCACGGUCCGCGCUAACUGUACCGC |
| | UGUGCCACCCACCG (SEQ ID NO: 510) |
| Ev3min16 | GGGAGAGAGGAAGAGGGAUGGGCACGGUCCAGCGCACUGUACCUGC |
| | UGUGCCACCCACCG (SEQ ID NO: 511) |
| Ev3min17 | GGGAGAGAGGAAGAGGGAUGGGCACGGUCCAGCGCUAACUGUACCU |
| | GCUGUGCCACCCACCG (SEQ ID NO: 512) |
| Ev3min18 | GGGAGAGGAAGAGGGAUGGGCACGGUCCAGCGCUAACUGUACCUGC |
| | UGUGCCACCCACCG (SEQ ID NO: 513) |
| Ev3min19 | GGGAGAGGAAGAGGGAUGGGCACGGUCCAGCGCUAACUGUACCUGC |
| | UGUGCCACCCACCG (SEQ ID NO: 514) |
| Ev3min20 | GAGGAAGAGGGAUGGGCACGGUCCAGCGCUAACUGUACCUGCUGUG |
| | CCACCCACCG (SEQ ID NO: 515) |

### Example 2—Sensitizing Cancer Cells with Nucleolin Aptamers

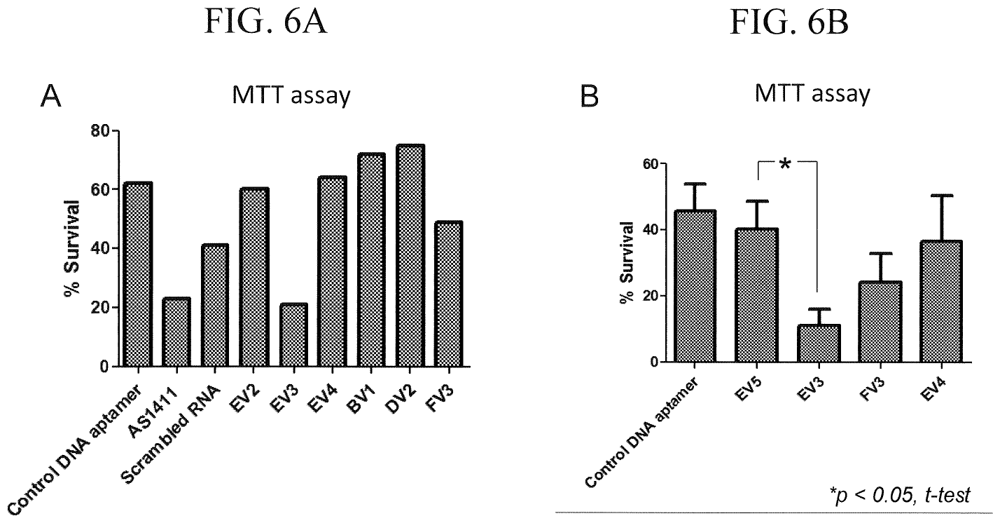

We next tested the ability of the nucleolin aptamer truncates Bv1, Ev3, Ev4, Dv2, and Fv3 to sensitize cancer cells that overexpress nucleolin on the cell surface to ionizing radiation (IR). We also included the Ev2 aptamer as a non-binding aptamer control. As shown in FIG. 6A, Ev3 appears to be a potent radiosensitizer, significantly decreasing post-IR survival in HCT116 p53-null cells. Further radiation sensitization studies showed that Ev3 decreased post-IR survival by approximately 5-fold in HCT116 p53-null cells compared to the aptamer control Ev5, which was used as a control due to its ability to bind nucleolin protein yet lack of radiosensitizing properties (FIG. 6B). Given that a large number of tumors lack functional p53, which is associated with resistance to therapy, it is encouraging that the specific nucleolin aptamer Ev3 can efficiently sensitize p53-null cells to IR.

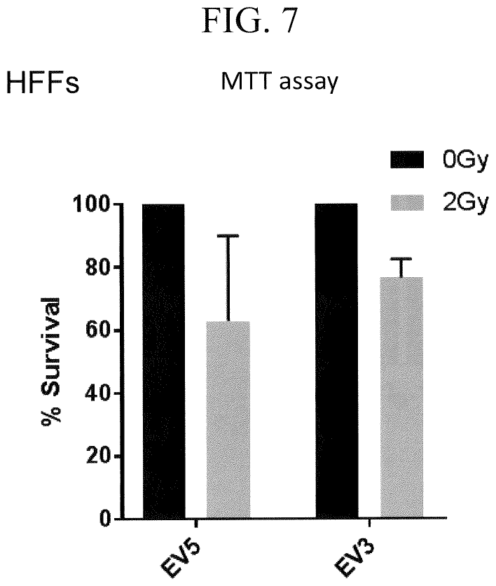

To determine whether the Ev3 aptamer's ability to sensitize cancer cells to ionizing radiation was specific to the nucleolin protein, we tested the aptamer on hTERT-immortalized HFF cells (FIG. 7). hTERT-immortalized HFF cells that do not express nucleolin on cell surface were treated with 5 ug of indicated aptamers and exposed to 2Gy IR 48h later. Cells were cultivated for 10d and survival was assessed by MTT assay. As seen in FIG. 7, Ev3 does not sensitize HFF (human foreskin fibroblasts) that do not express nucleolin on cell surface to radiation.

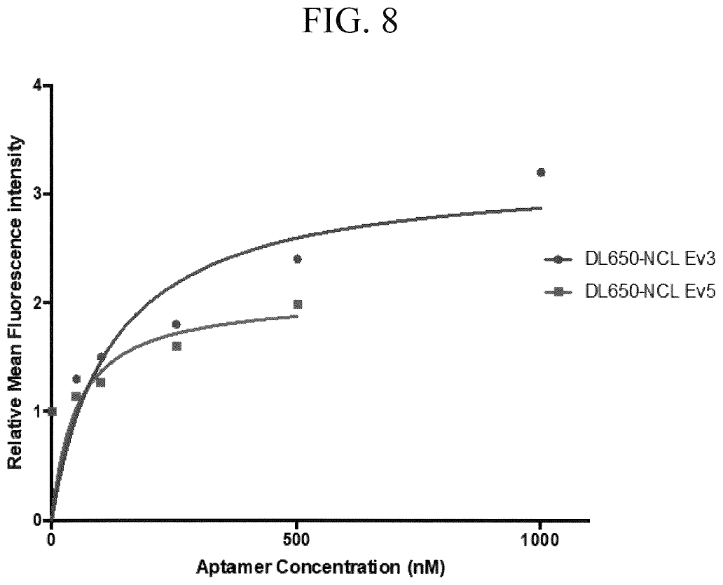

To determine the Ev3 and Ev5 aptamers could bind nucleolin expressed on a cell surface in a concentration-dependent manner, we performed a flow cytometry analysis with HCT116 p53−/− cells. Flow cytometry analysis of MFI (mean fluorescence intensity) of DL650-labeled Ev3 and Ev5 after incubation of HCT116 p53−/− cells with indicated aptamer concentrations. As shown in FIG. 8 and Table 9, Ev3 and Ev5 bind to nucleolin expressed on the cell surface in a concentration dependent manner.

TABLE 9
Ev3 and Ev5 Binding Data

| One site binding (hyperbola) | DL650-NCL | DL650-NCL |
|---|---|---|
| Best-fit values | Ev3 | Ev5 |
| Bmax | 3.214 | 2.064 |
| Kd | 119.2 | 50.7 |

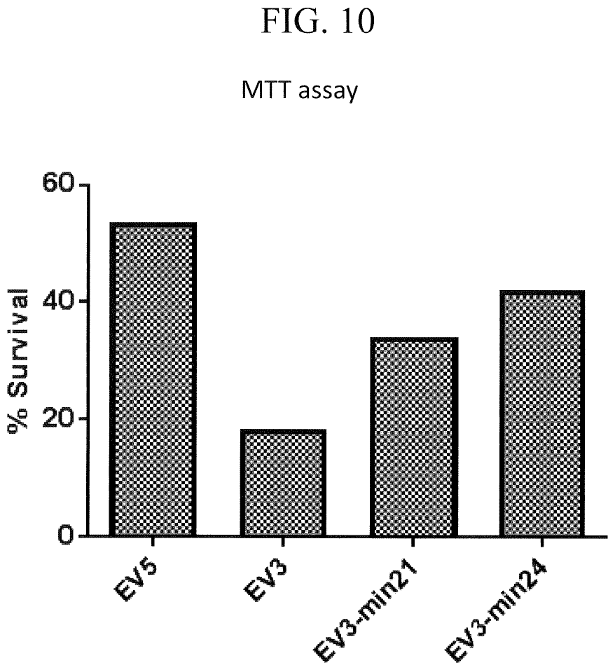

To determine whether the EV3 aptamer could be truncated without affecting its radiosensitization function, we tested some Ev3 aptamer truncates (FIG. 10). HCT 116 p53−/− colon cancer cells were treated with 5 ug of indicated full-length aptamers or Ev3 truncates and exposed to 2Gy IR 48h later. Cells were cultivated for 10d and survival was assessed by MTT assay. FIG. 10 shows truncation of Ev3 resulted in reduced activity as radiosensitizer.

The Ev3 nucleolin aptamer has the potential for clinical application as a cancer-specific radio- and chemosensitizer and could improve the current regimens of cancer therapy. Further, the aptamer can be radiolabeled for use as a DNA damaging agent that will preferentially target tumors and simultaneously blunt the ability of the tumor cell to repair the radiation damage, thus enhancing the sensitivity of the tumor to the radioisotope.

### Example 3—Predicted Secondary Structures for Nucleolin Aptamers

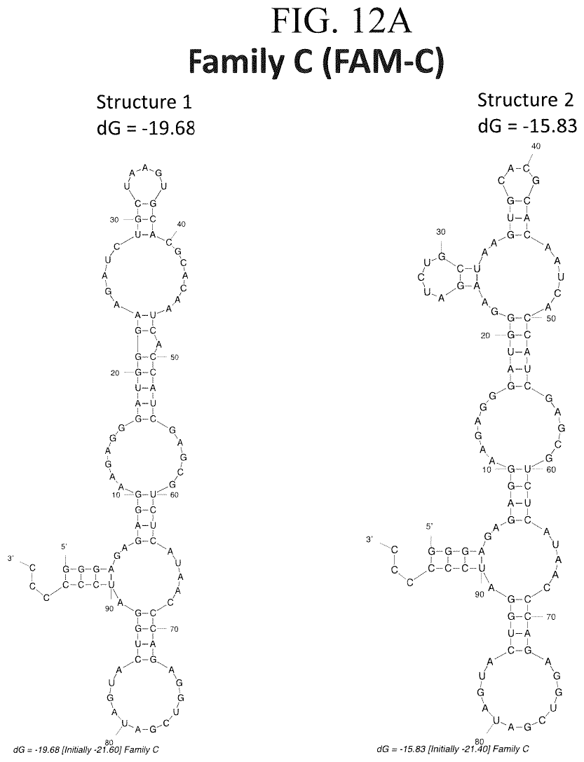

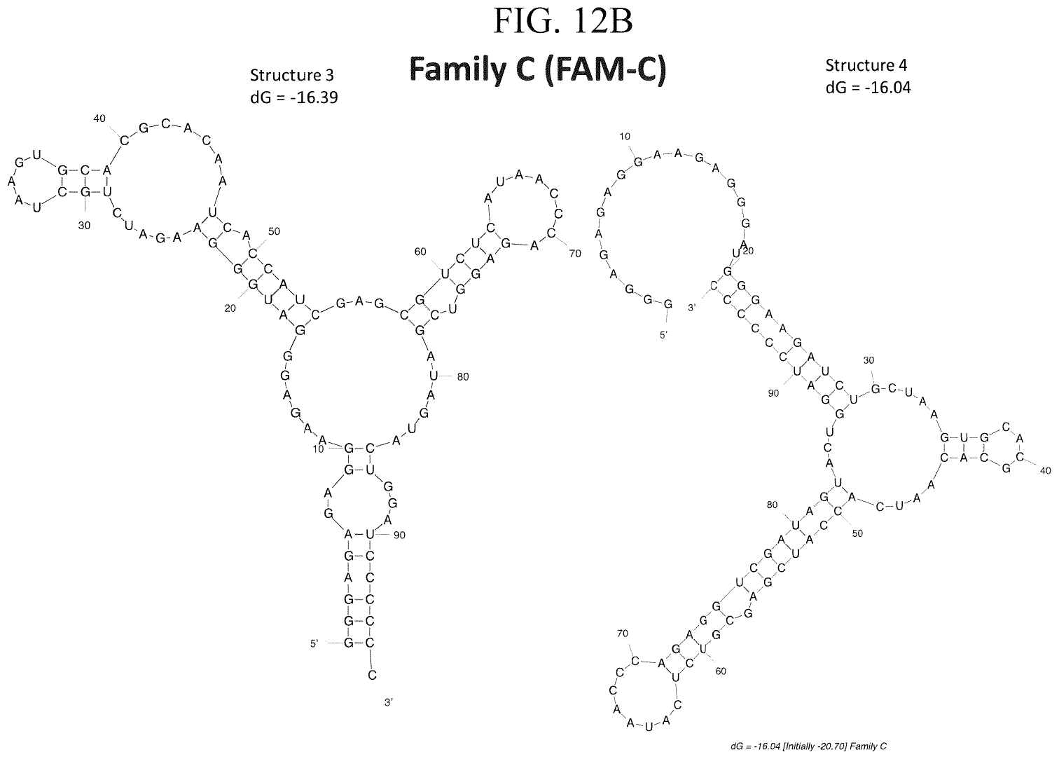

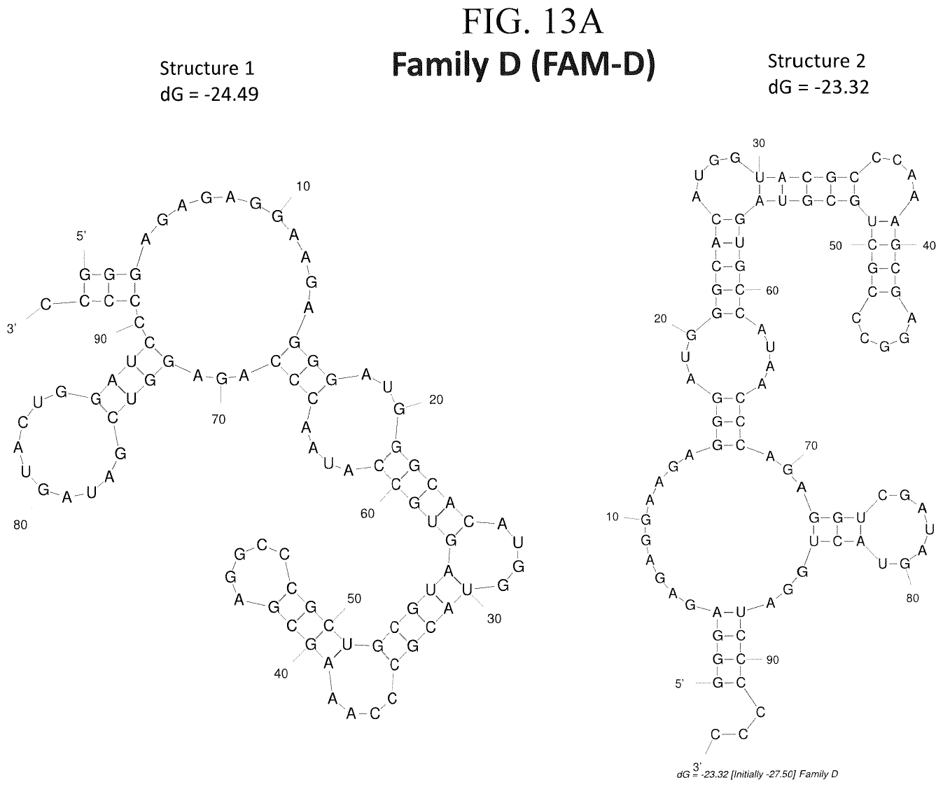

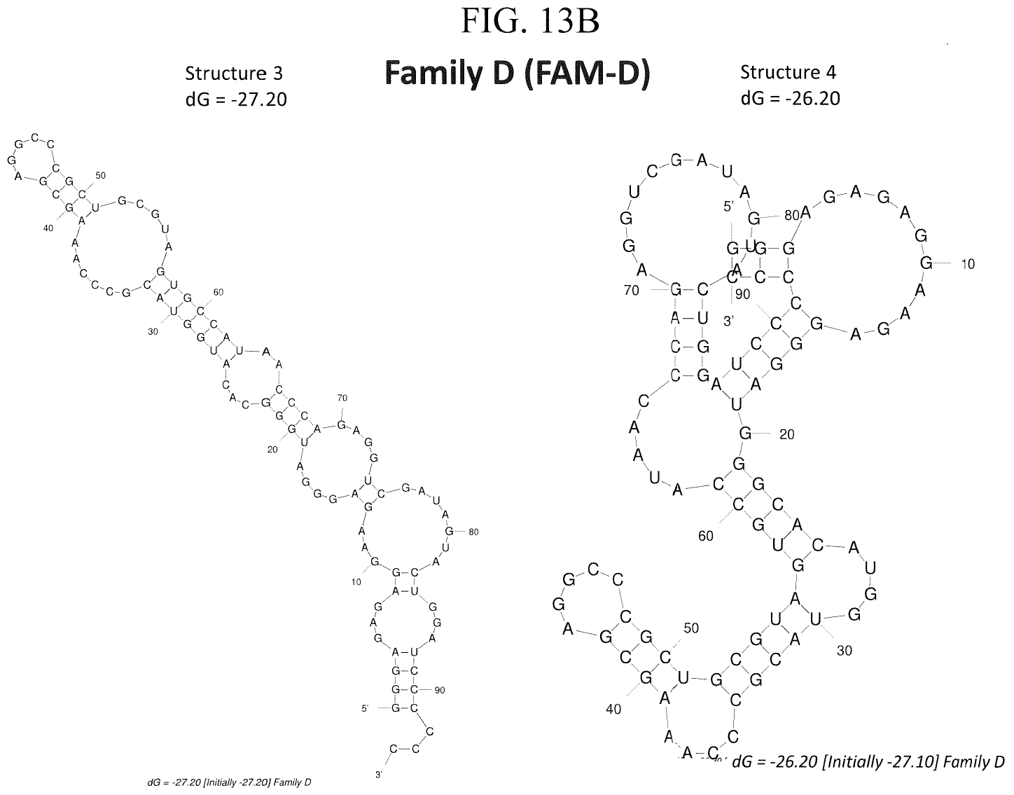

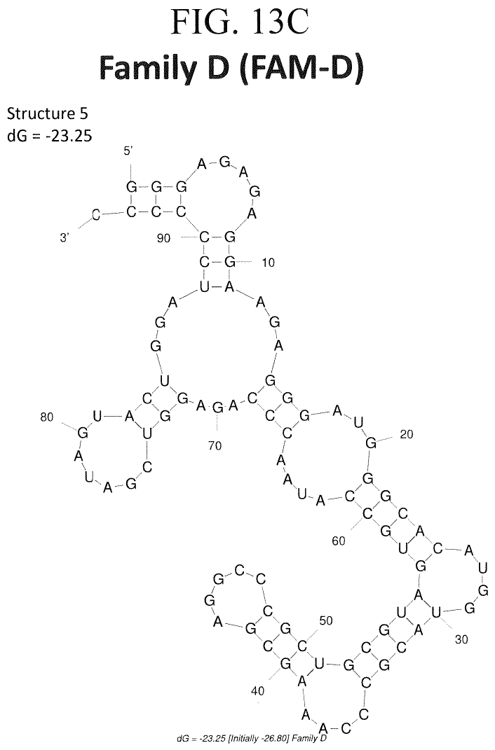

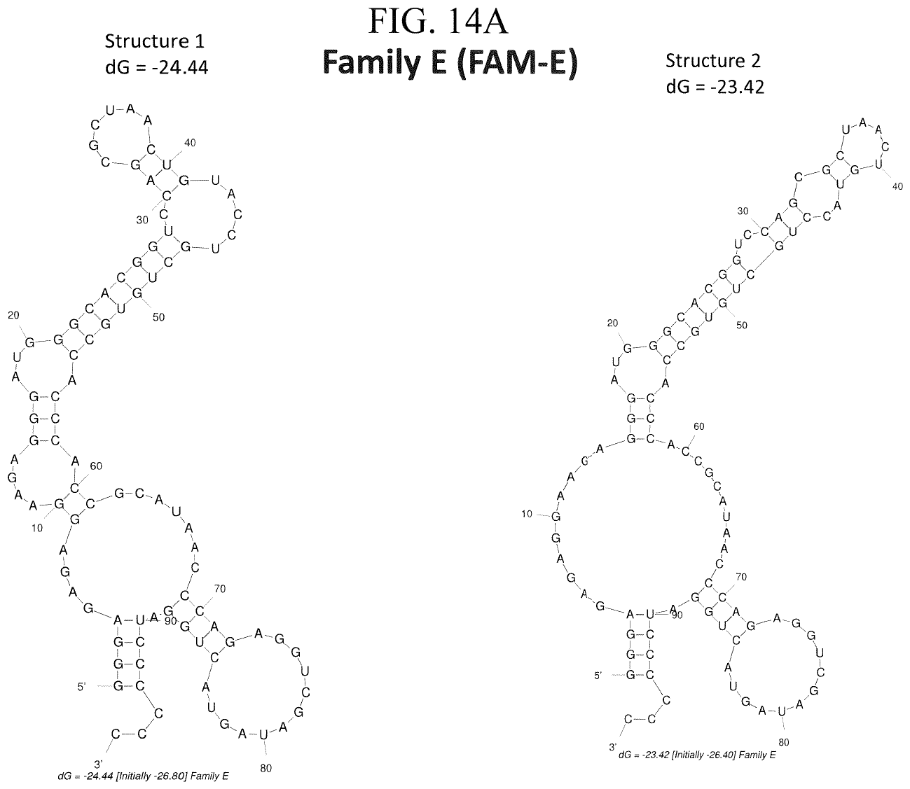

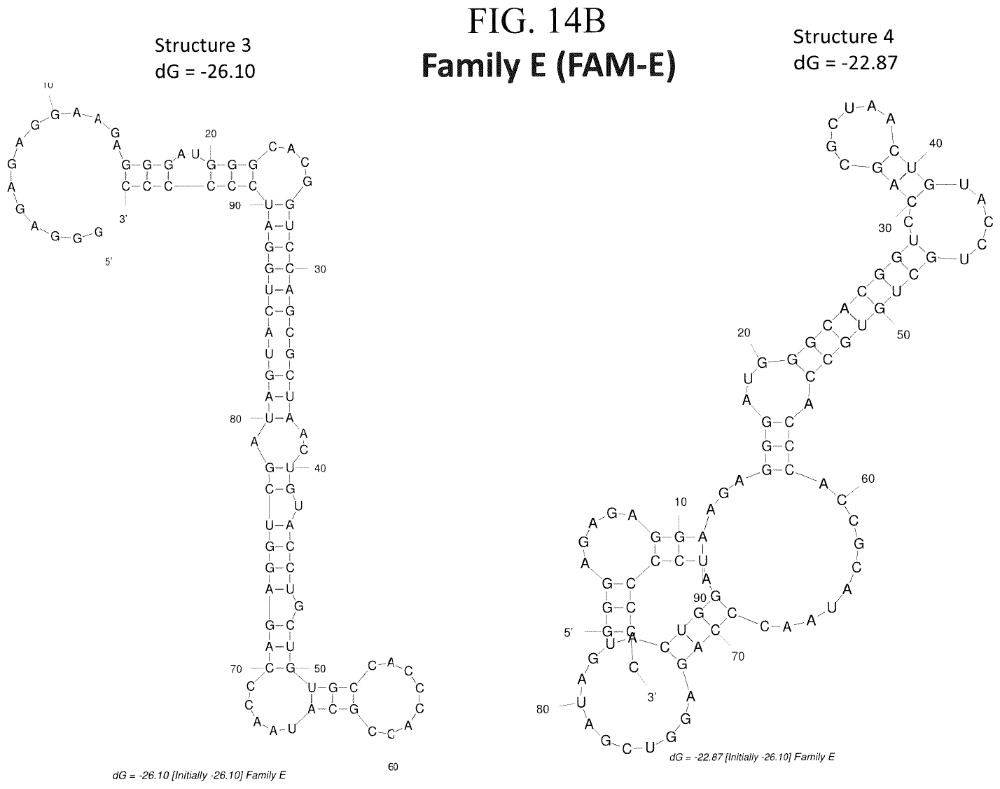

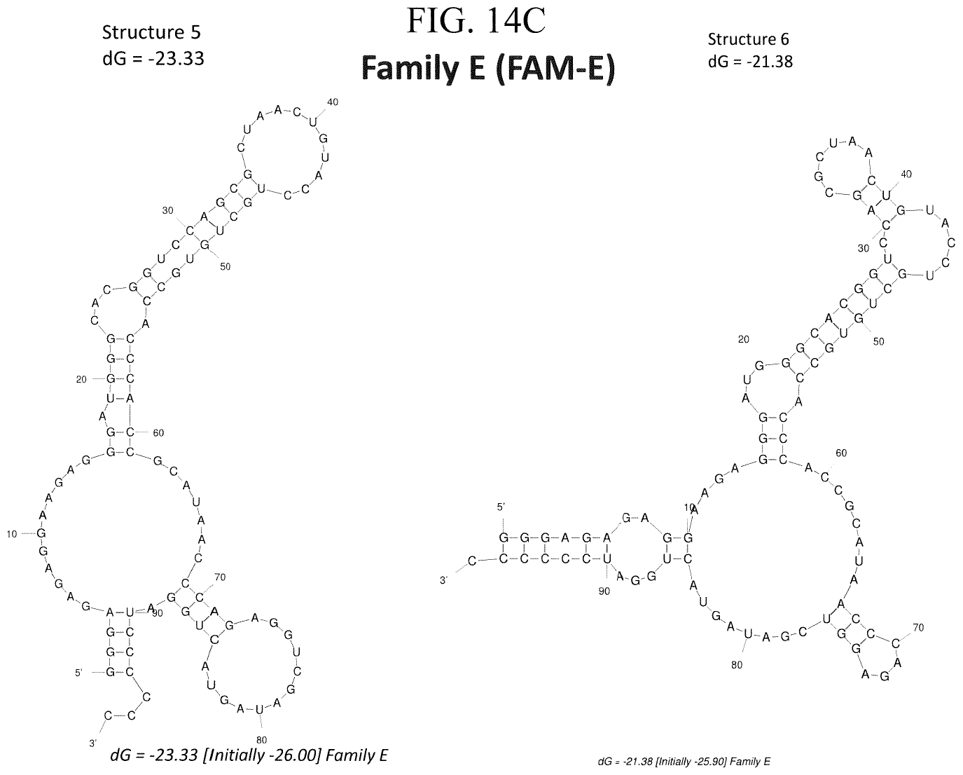

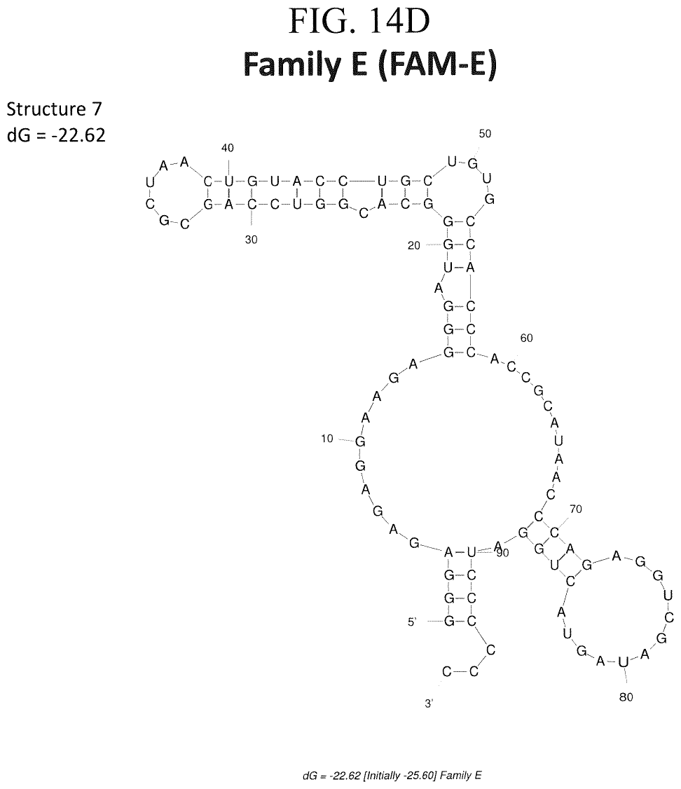

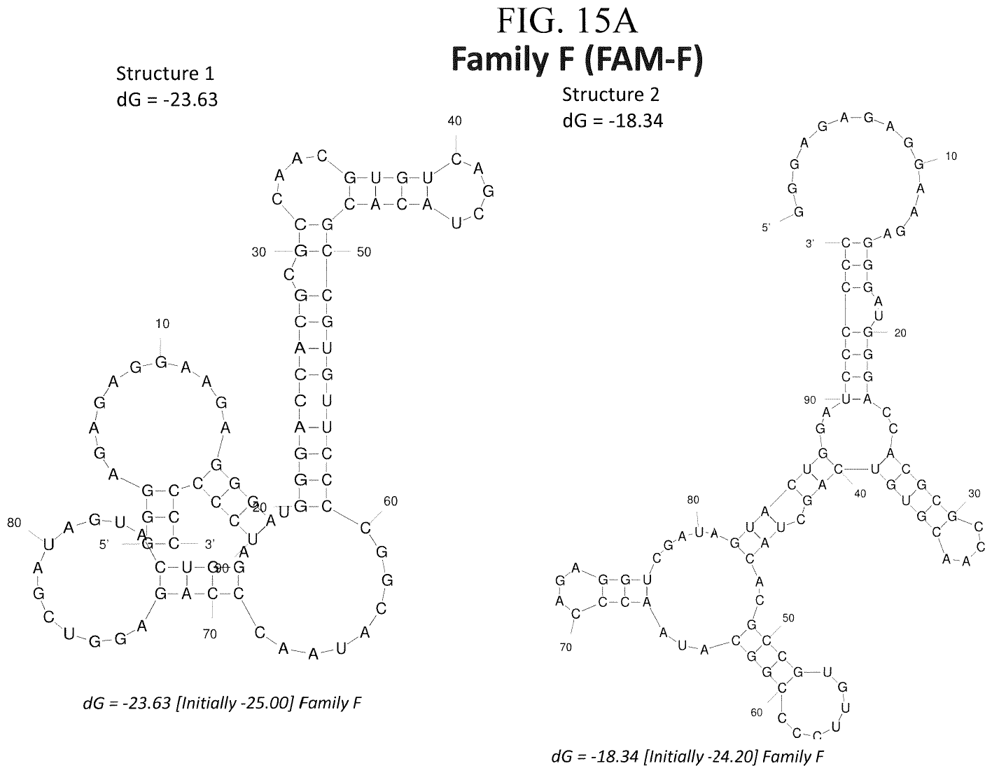

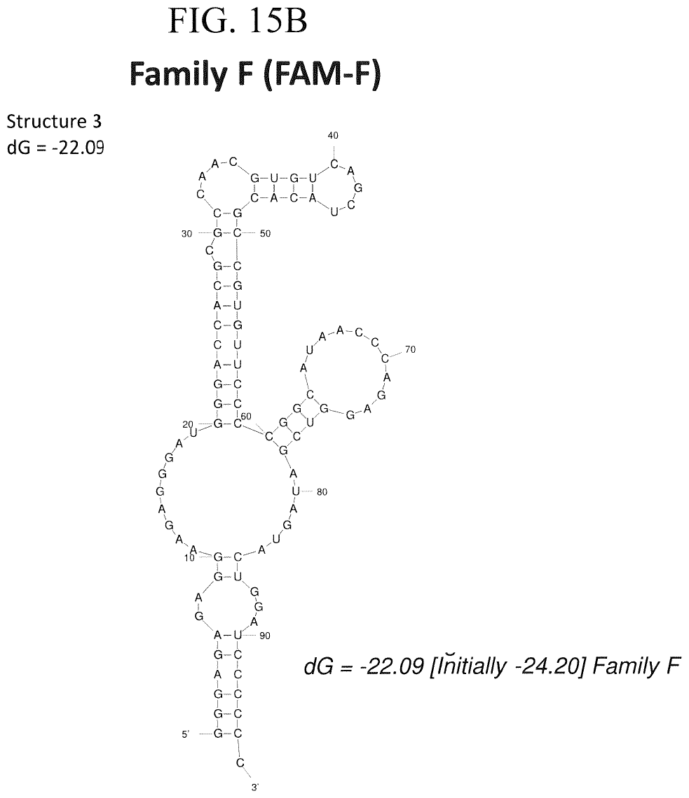

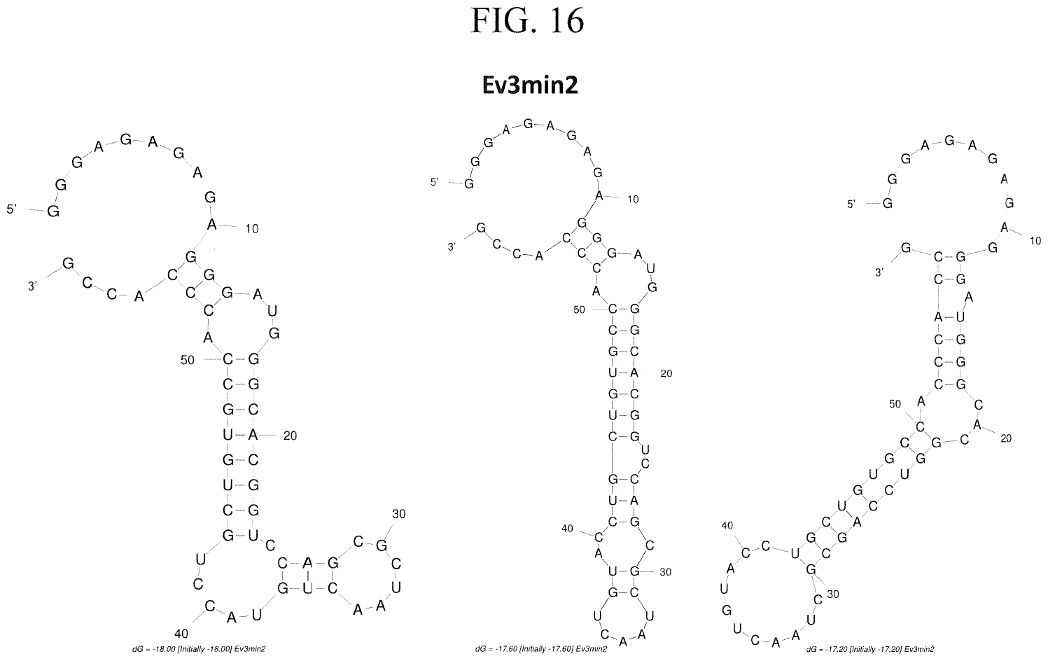

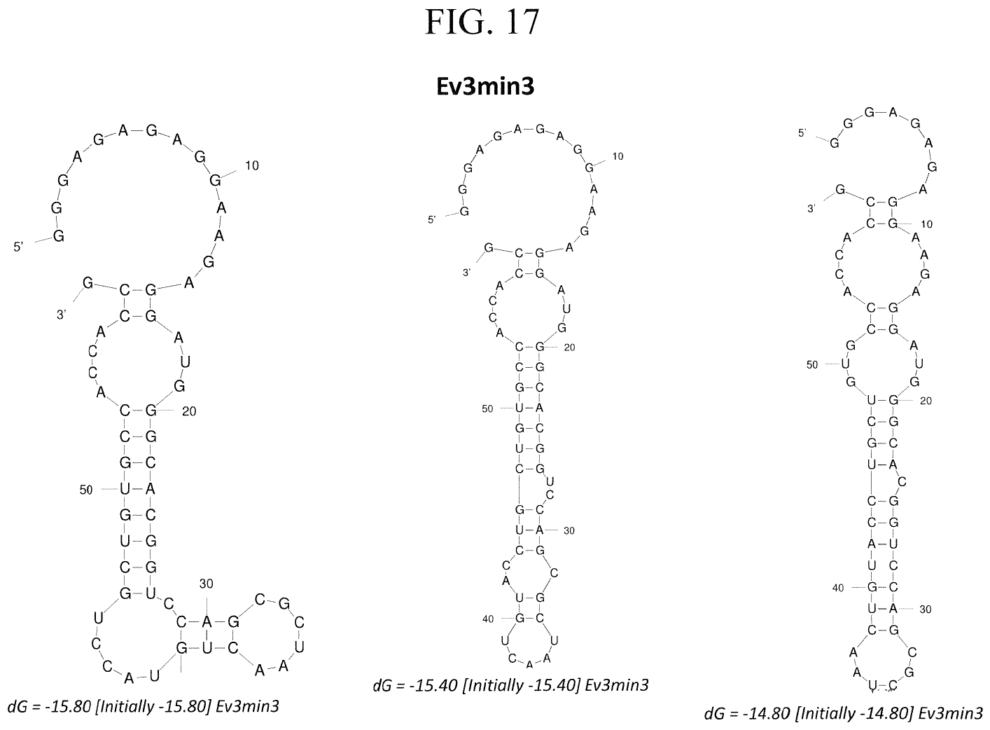

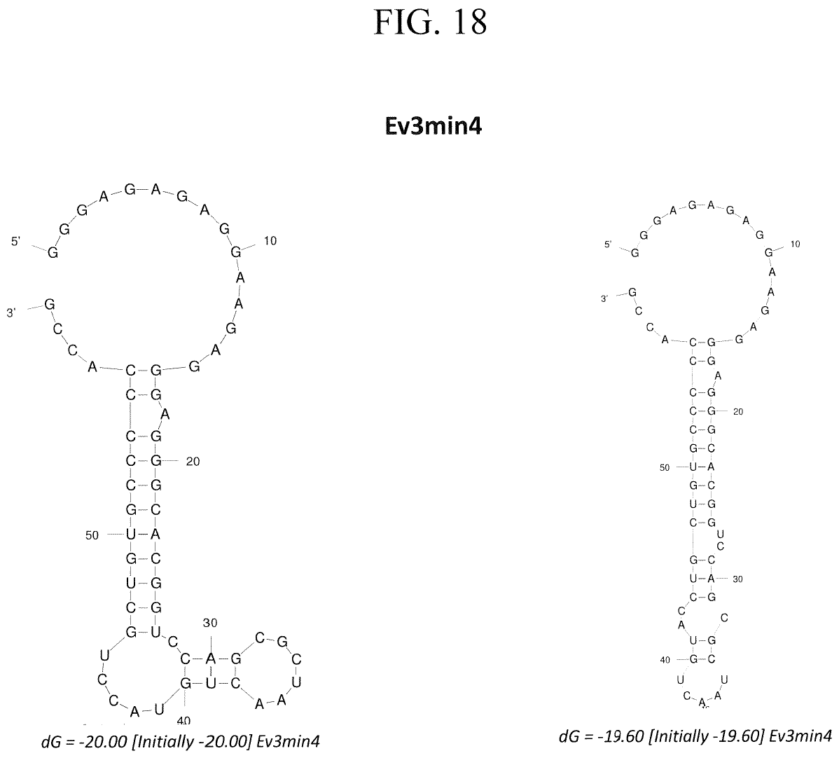

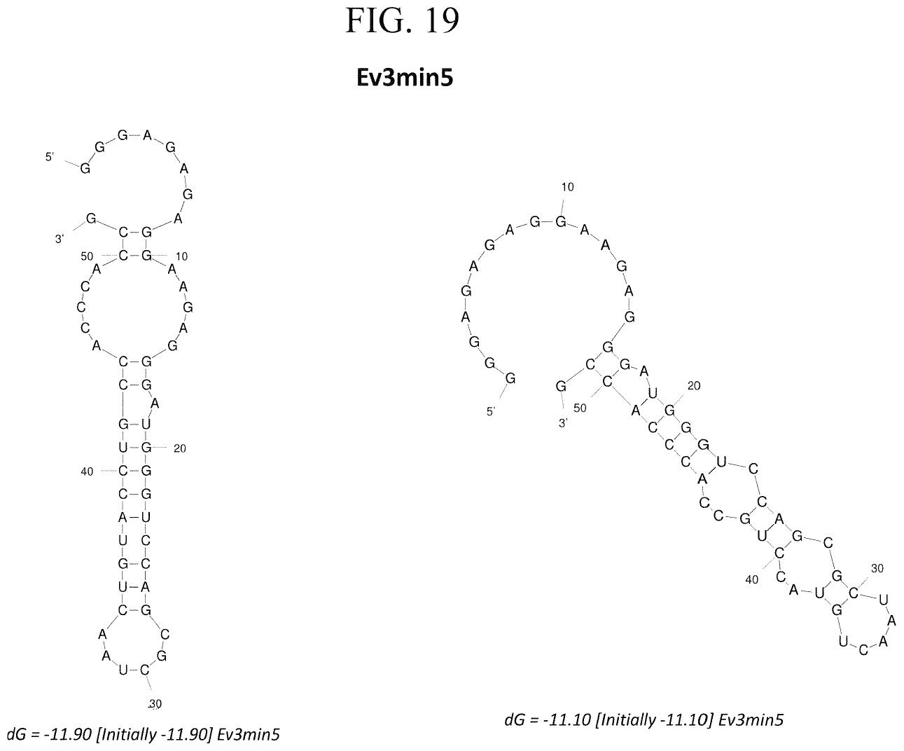

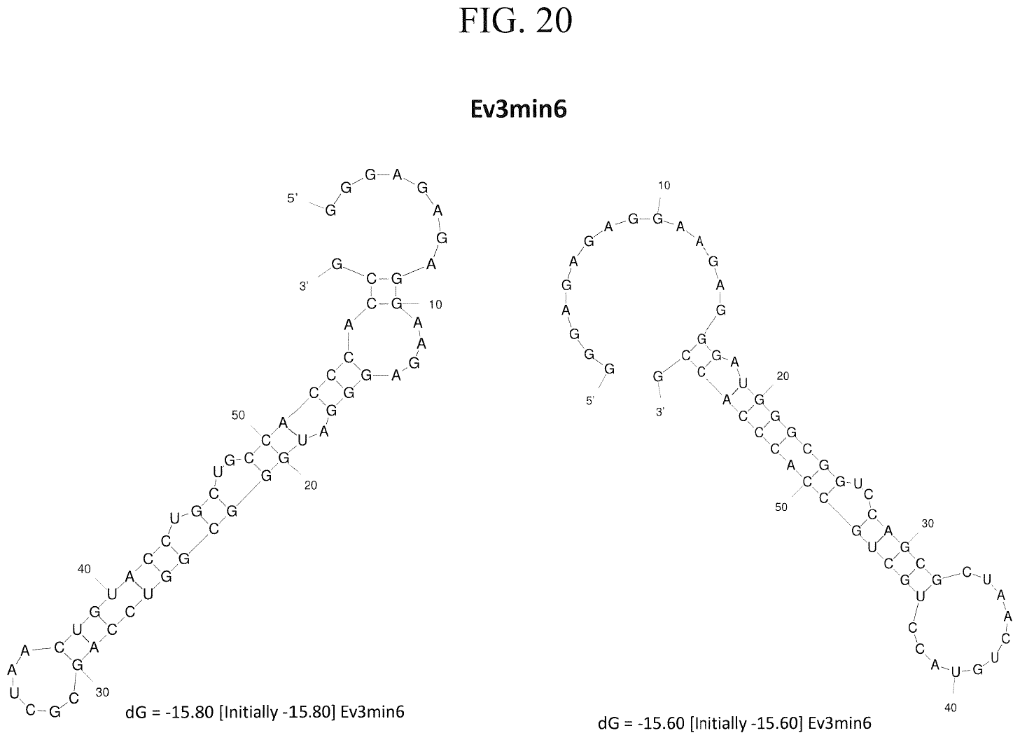

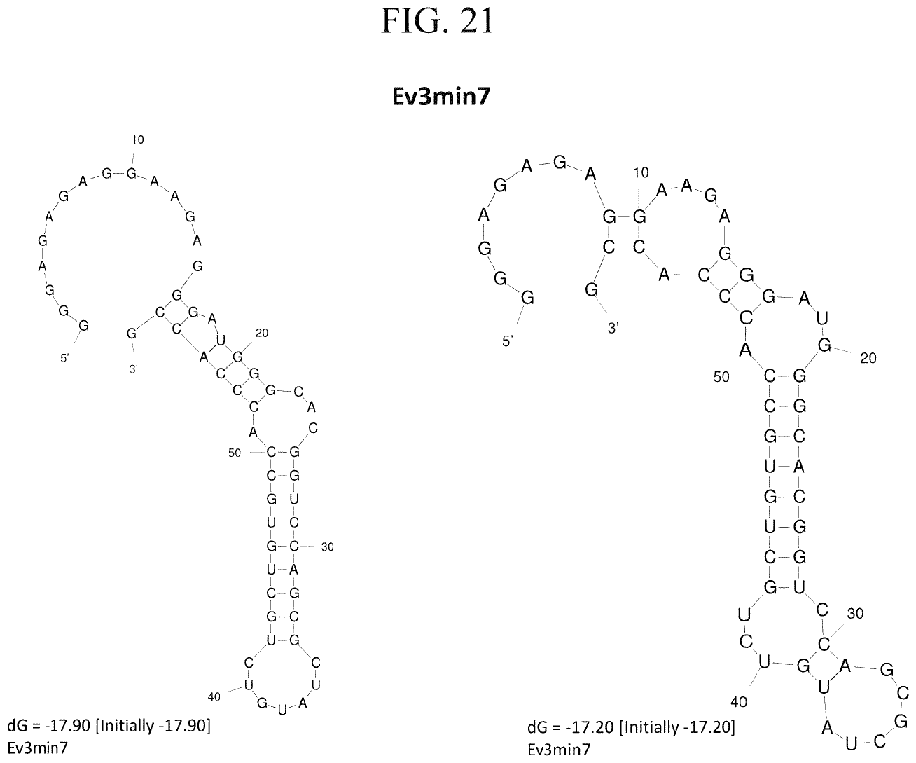

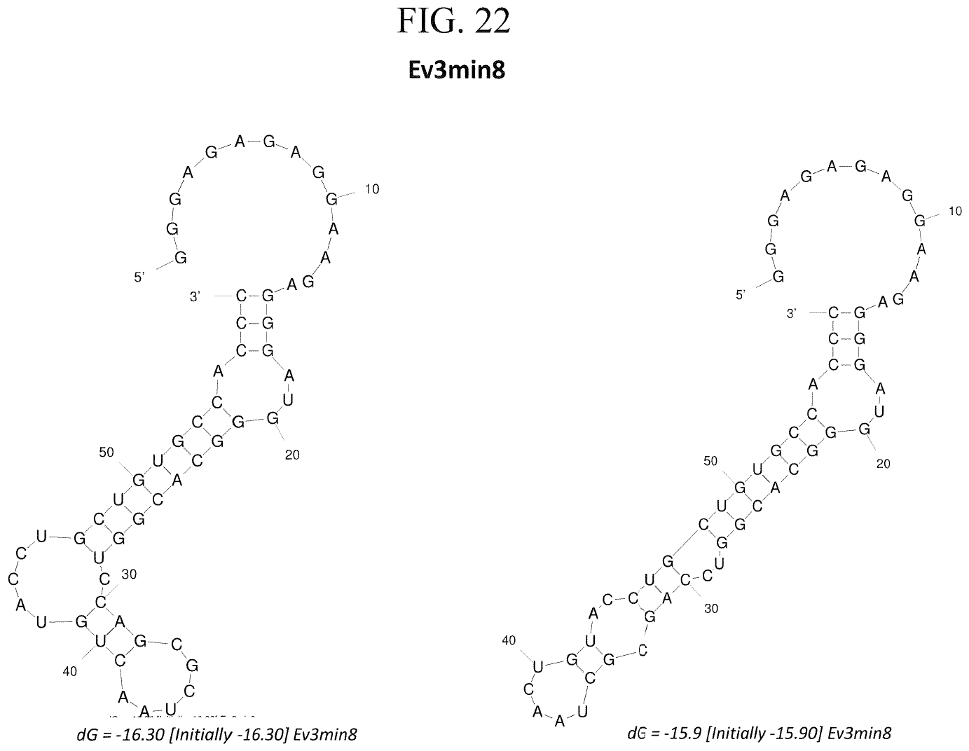

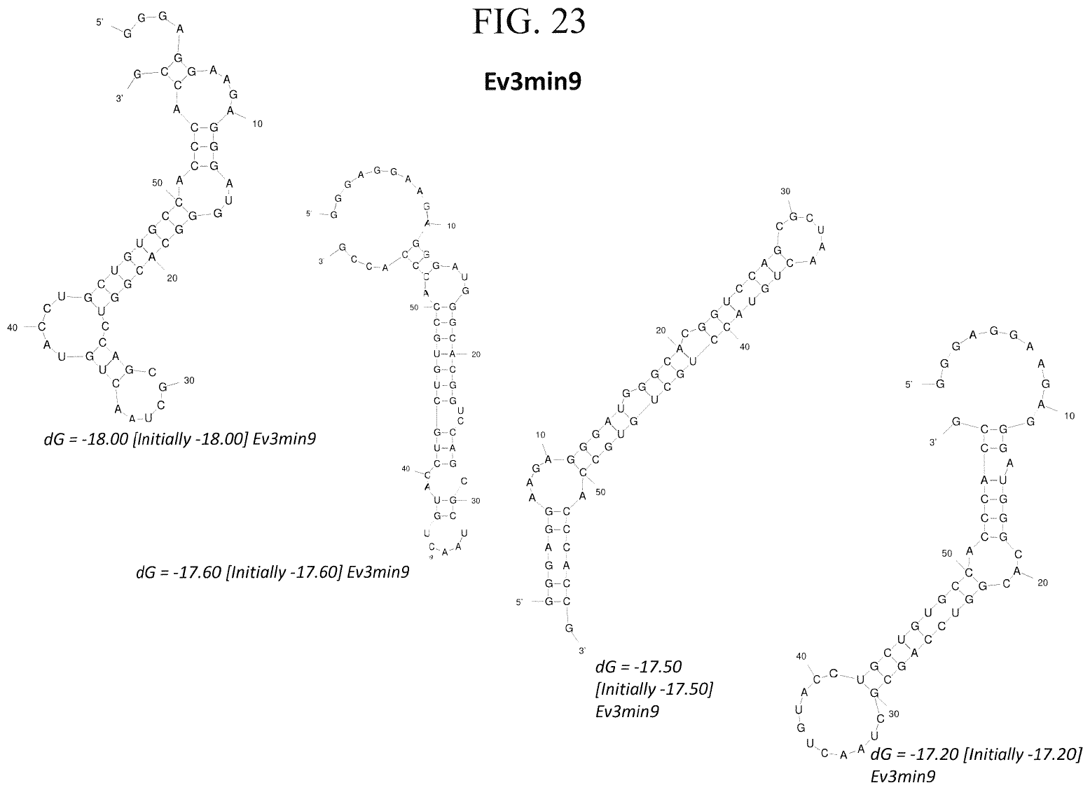

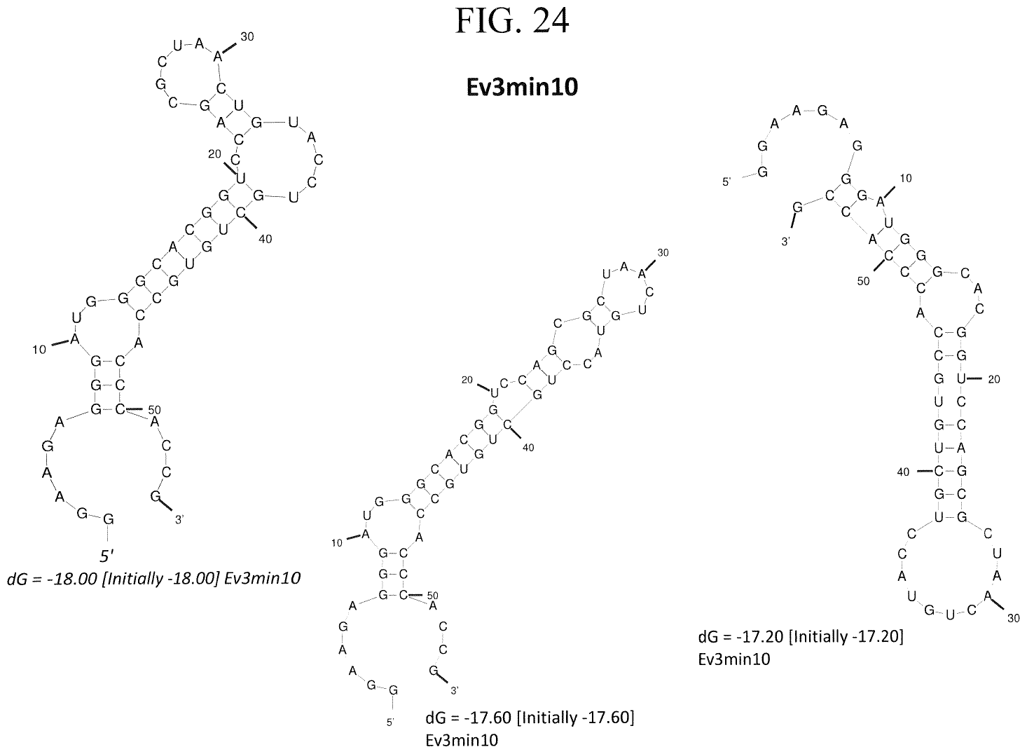

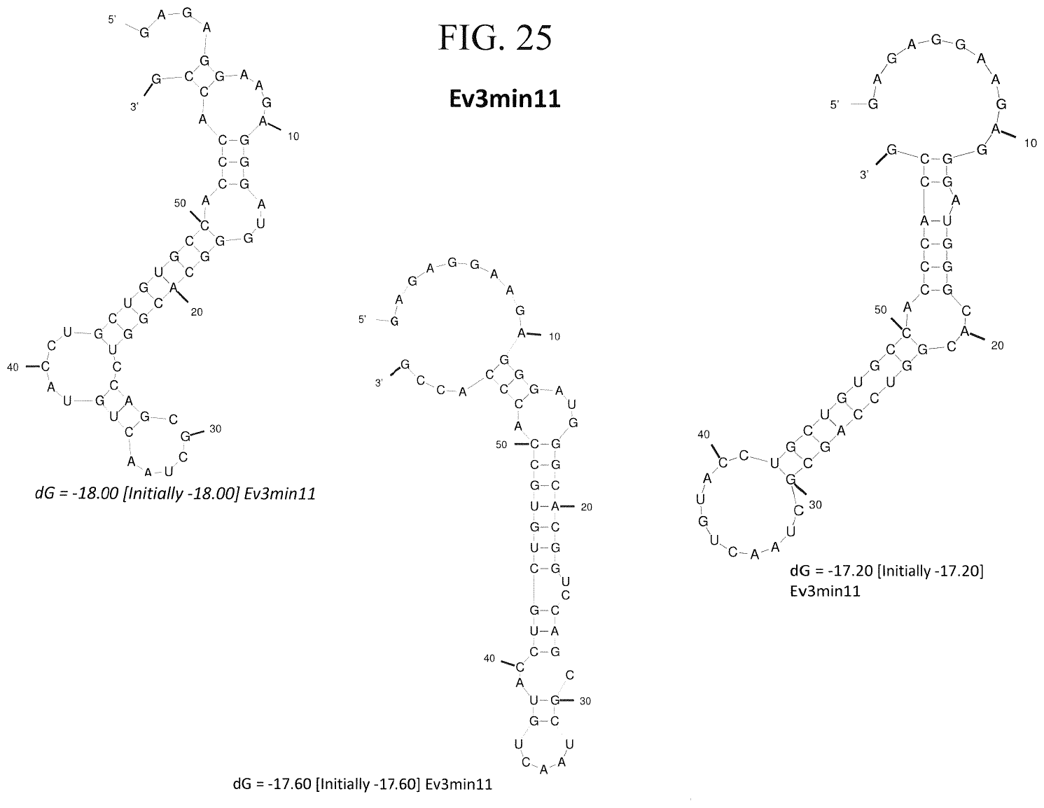

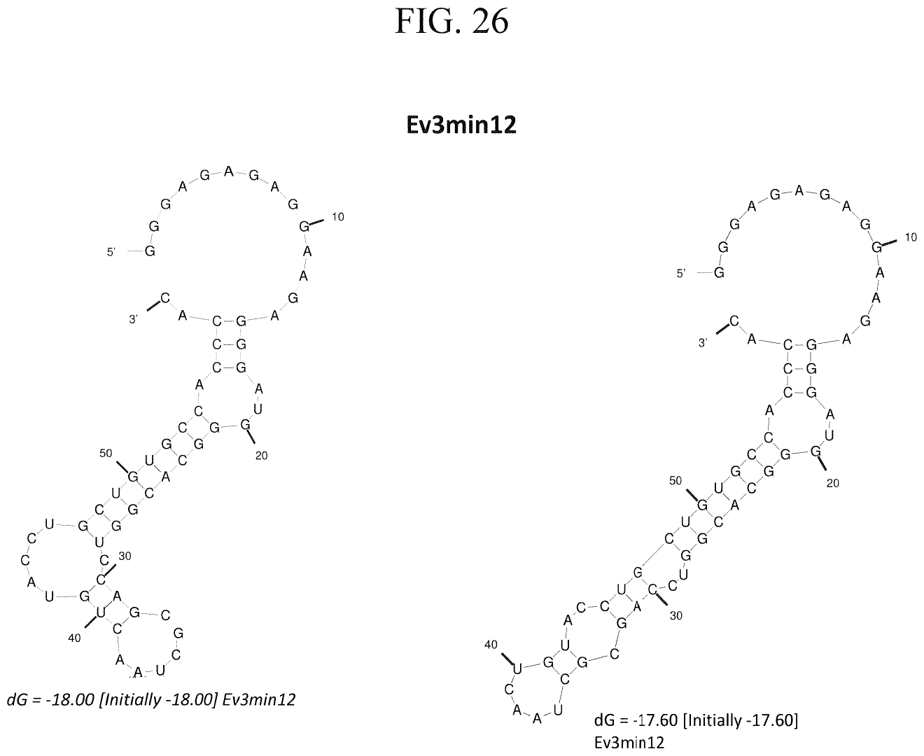

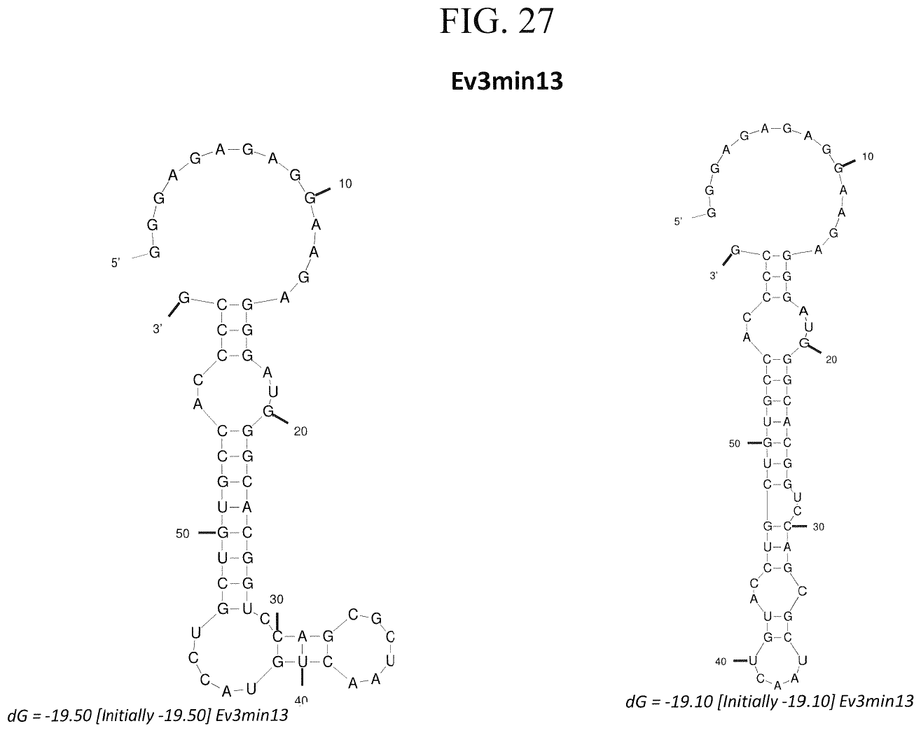

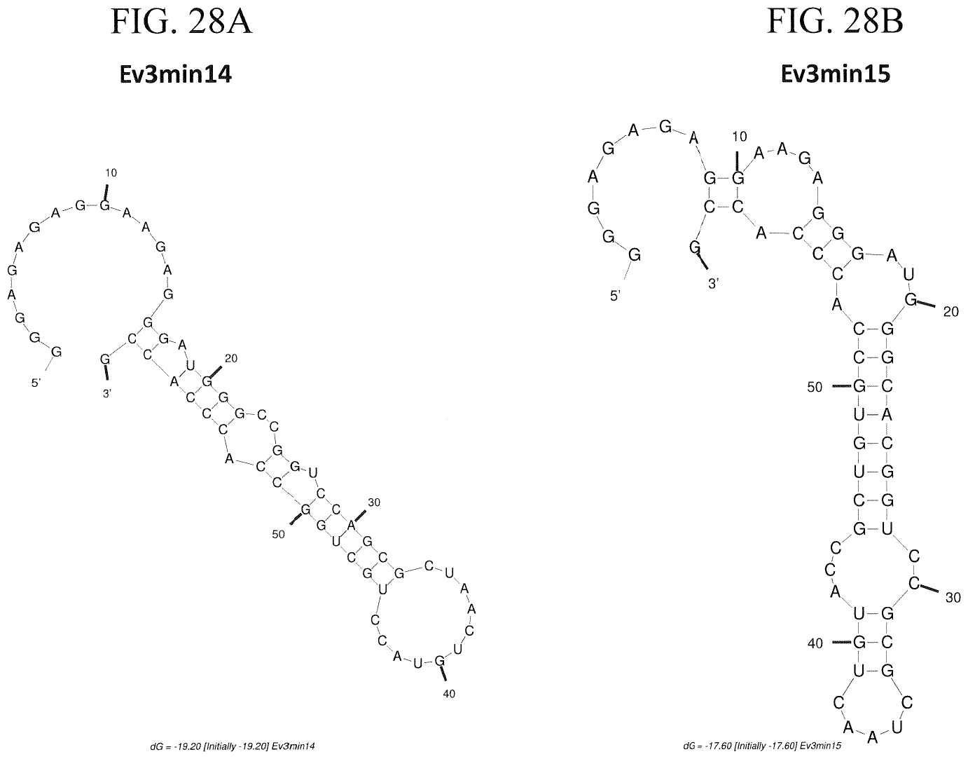

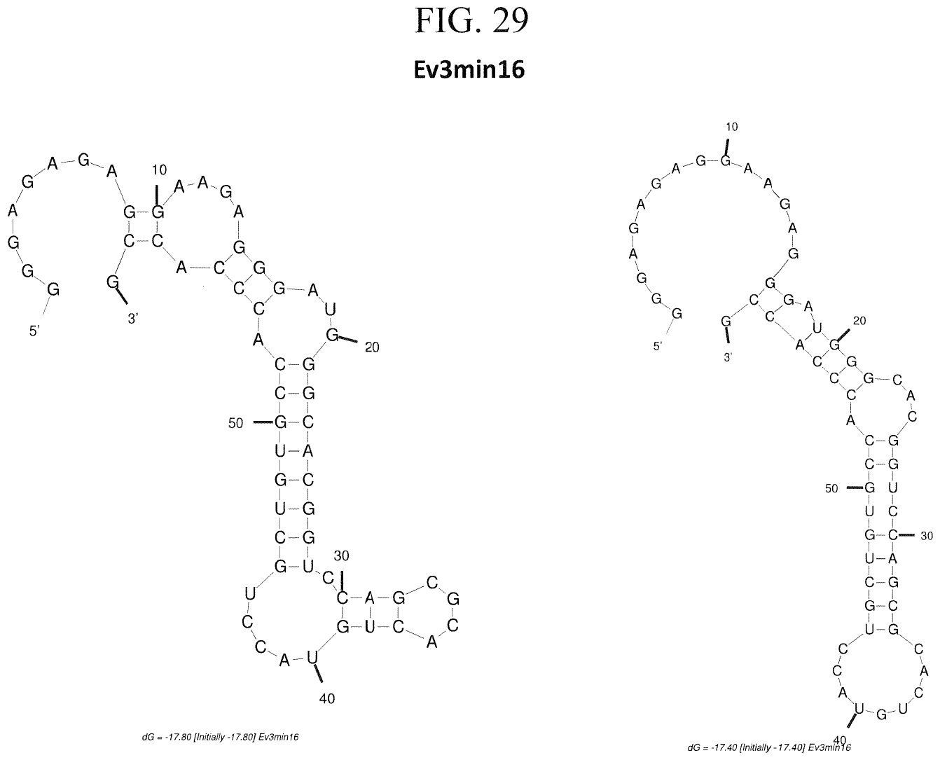

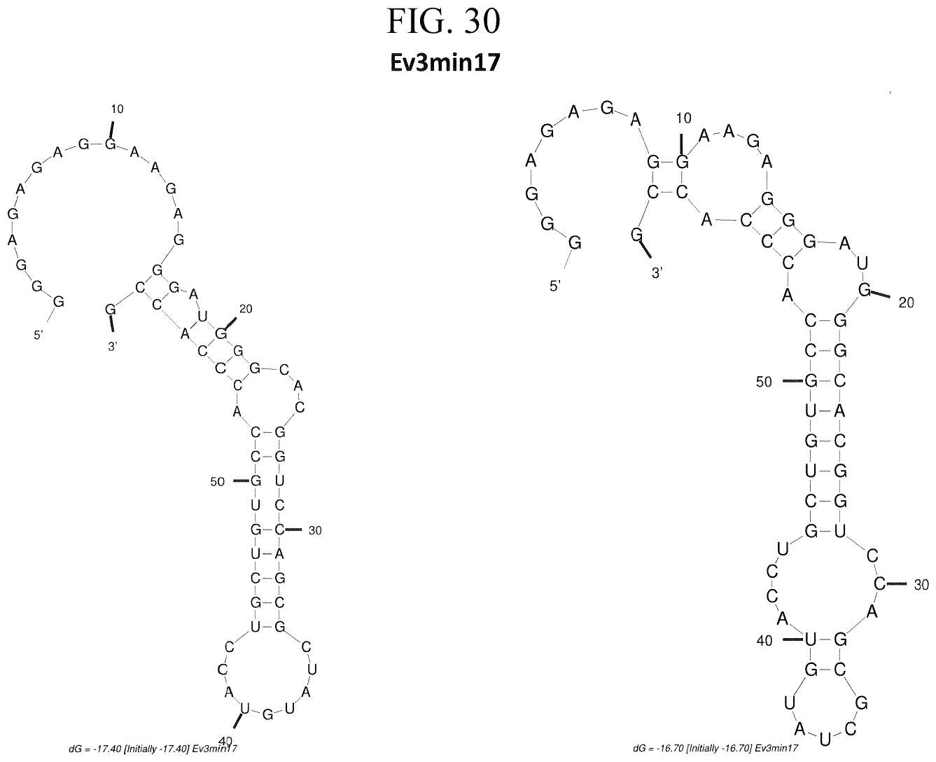

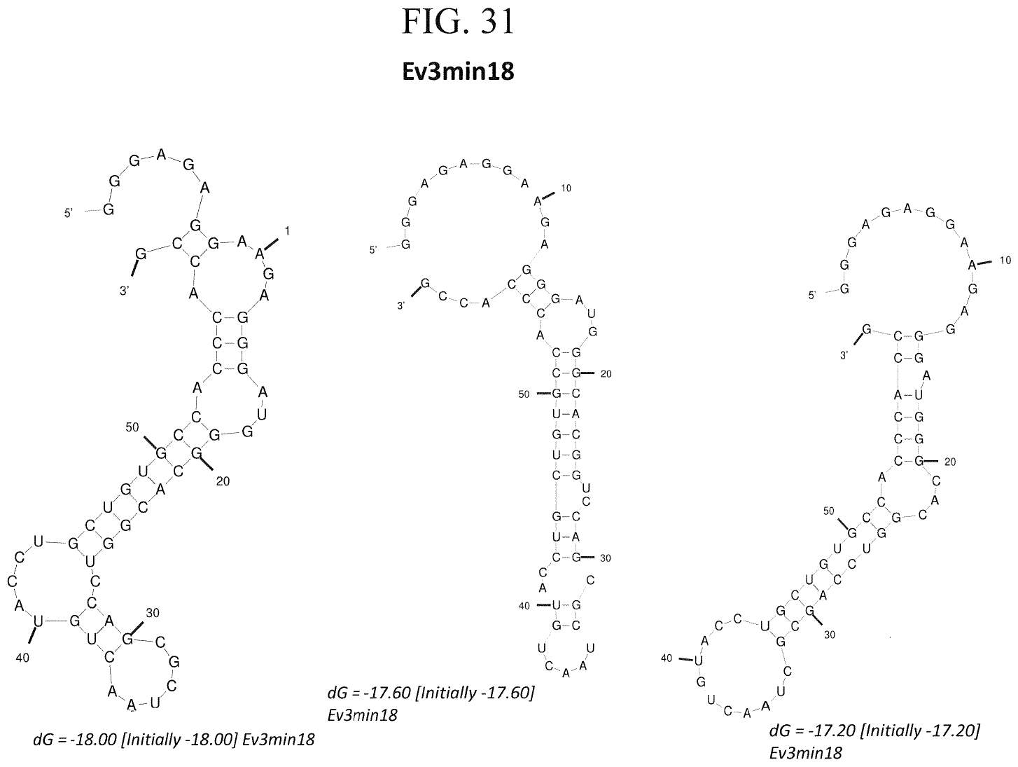

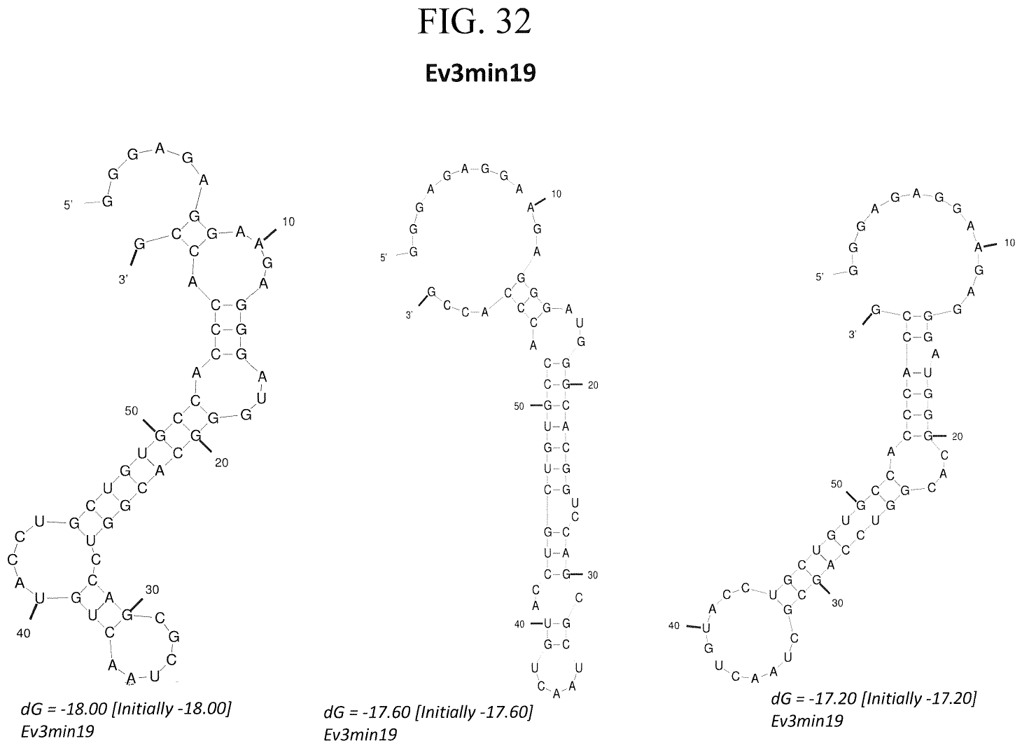

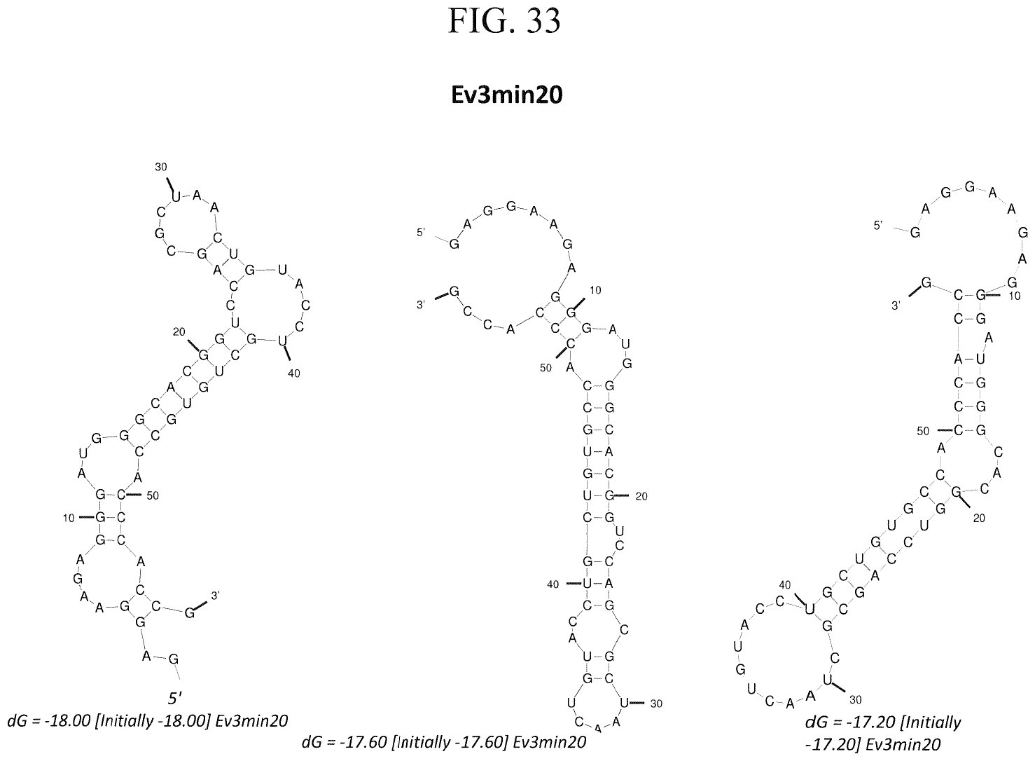

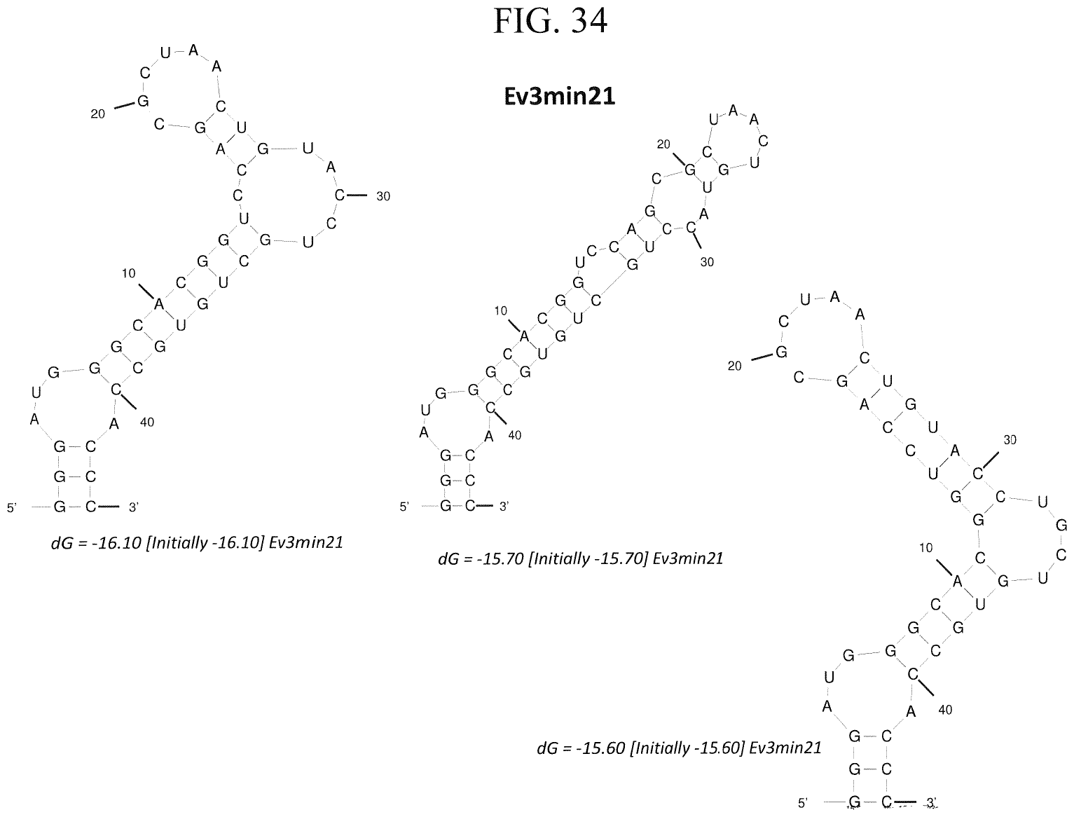

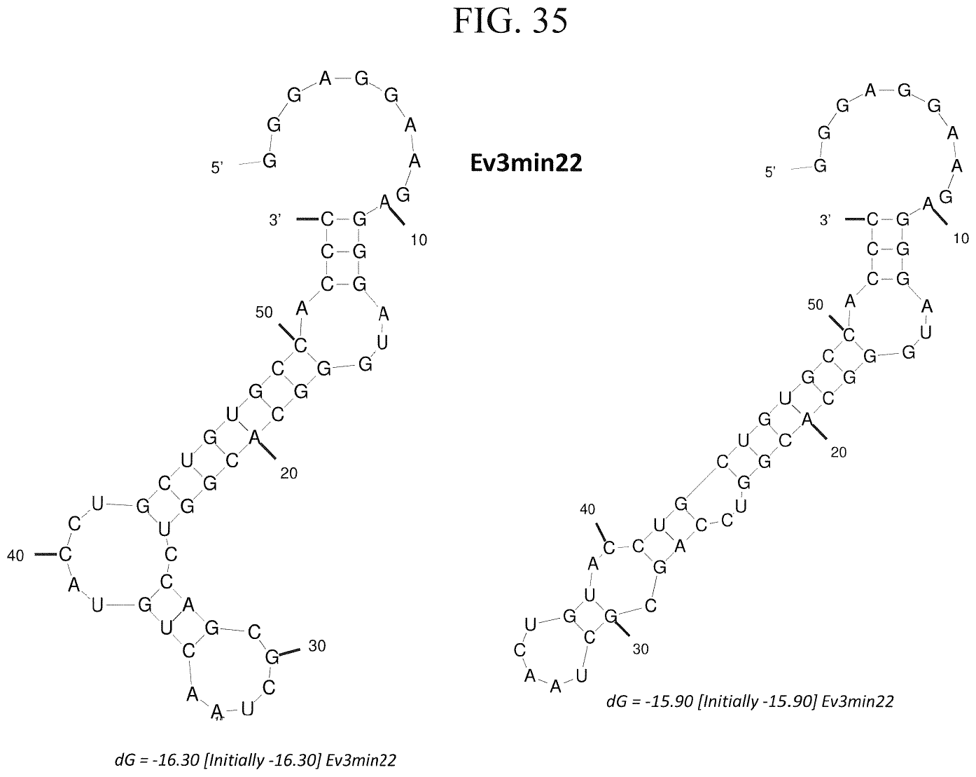

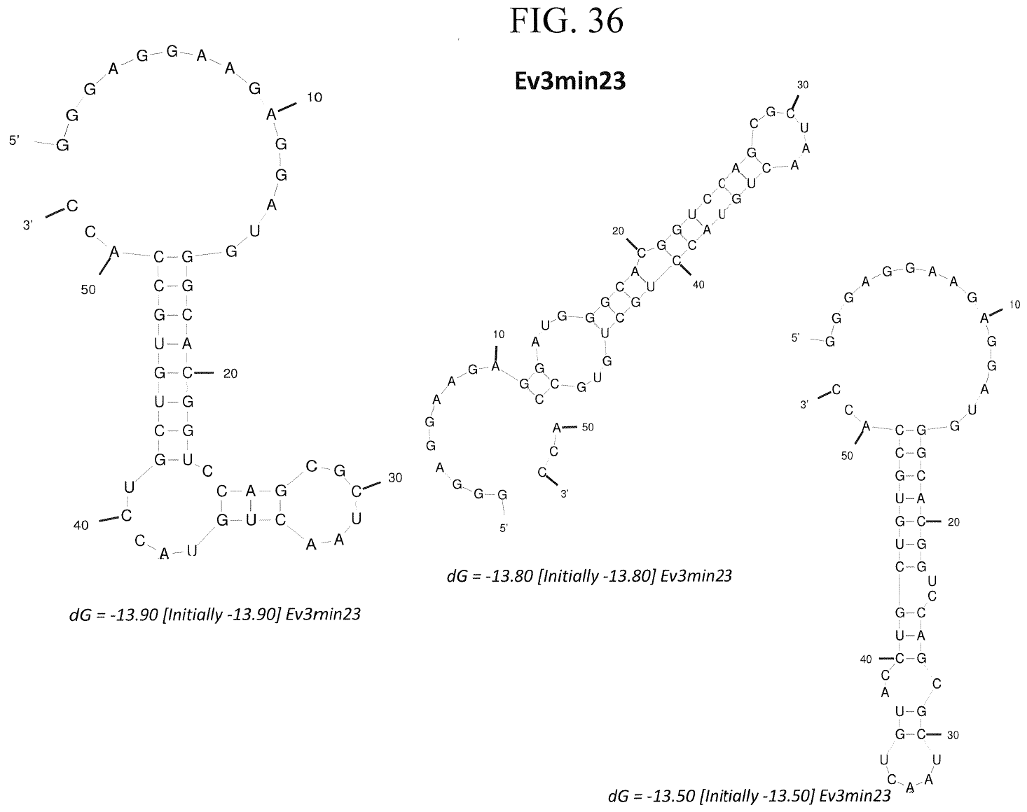

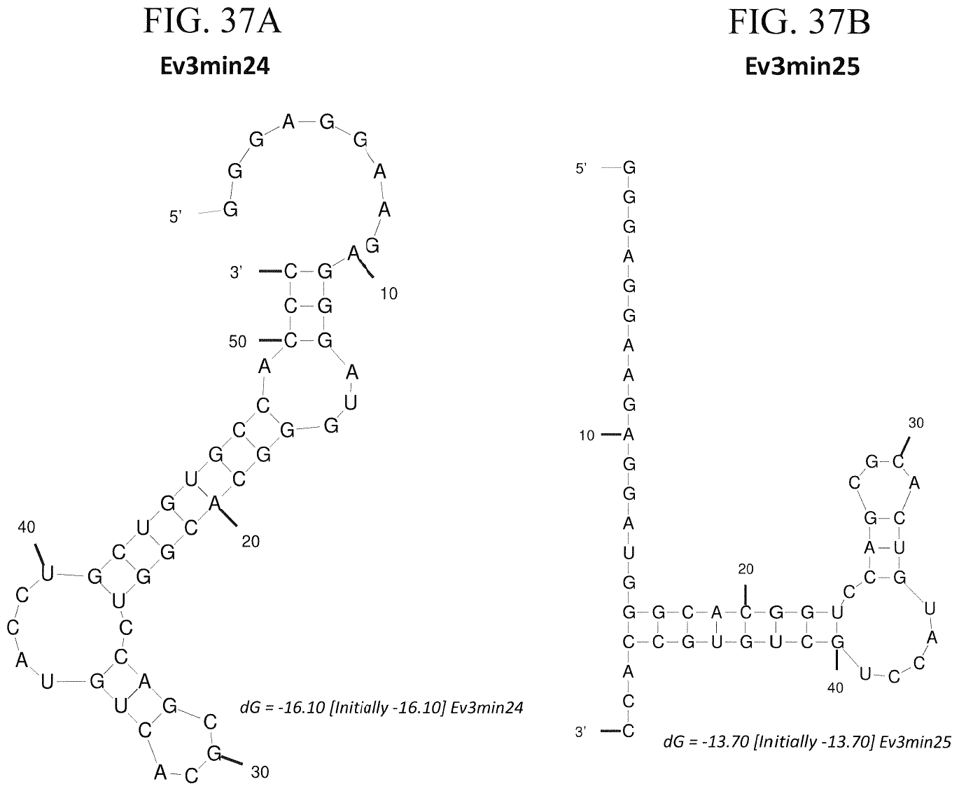

Predicted secondary structures for nucleolin aptamers were generated using the mfold Web Server RNA Folding Form. Predicted structures for representative aptamers from families B, C, D, E, and F are shown in FIGS. 11A-11B, 12A-12B, 13A-13C, 14A-14D, and 15A-15B. Predicted structures for Ev3 truncates (Ev3.min2-25) are shown in FIGS. 16-37.

1. An aptamer comprising
a polynucleotide having at least 80% sequence identity to any one of SEQ ID NOS: 1-12 22-473, 475-479, 481, 483-484, 486, 490-502, or 504-515,
wherein the polynucleotide comprises an unmodified form or comprises a modified form comprising at least one nucleotide base modification.

2. The aptamer of claim 1, wherein the aptamer comprises a polynucleotide having at least 90% sequence identity to 5′-GGGAGAGAGGAAGAGGGAUGGG (SEQ ID NO: 491)-A Variable Region-CAUAACCCAGAGGUCGAUAGUACUGGAUCCCCCC (SEQ ID NO: 492)-3′, wherein the variable region comprises any one of SEQ ID NOS: 22-473 or a portion thereof.

3. The aptamer of claim 1, wherein the aptamer comprises a polynucleotide having at least 90% sequence identity to SEQ ID NO: 480 (Ev3 Aptamer).

4. The aptamer of claim 1, wherein the dissociation constant (K_{D}) of the aptamer for a nucleolin protein is less than 100 nanomolar (nM).

5. The aptamer of claim 1, wherein the polynucleotide comprises an RNA polynucleotide.

6. The aptamer of claim 1, wherein the polynucleotide comprises a modified form comprising at least one nucleotide base modification selected from the group consisting of a 2′fluoro modification, a 2′O-methyl modification, a 5′ modification, and a 3′modification.

7. The aptamer of claim 1, wherein the polynucleotide comprises a 5′ linker and/or a 3′ linker.

8. The aptamer of claim 1, wherein the polynucleotide further comprises an agent.

9. The aptamer of claim 8, wherein the agent is a stability agent selected from the group consisting of polyethylene glycol (PEG), cholesterol, albumin, and Elastin-like polypeptide or a reporter moiety.

10. (canceled)

11. The aptamer of claim 9, wherein said reporter moiety is selected from the group consisting of a fluorophore moiety, an optical moiety, a magnetic moiety, a radiolabel moiety, an X-ray moiety, an ultrasound imaging moiety, a photoacoustic imaging moiety, a nanoparticle-based moiety, and a combination of two or more of the reporter moieties.

12. The aptamer of claim 8, wherein the polynucleotide and the agent are linked by a covalent bond or a tag system.

13. (canceled)

14. (canceled)

15. A dimer, trimer, or tetramer comprising the aptamers of claim 1.

16. (canceled)

17. A method for treating cancer in a subject comprising administering to the subject a therapeutically effective amount of the compositions of claim 1.

18. The method of claim 17, further comprising administering a chemotherapeutic agent or radiation therapy to the subject.

19. The method of claim 18, wherein the composition is administered prior to the administration of the chemotherapeutic agent or the radiation therapy.

20. (canceled)

21. The method or use of claim 17, wherein the cancer is colon cancer.

22. The method of claim 17, wherein the subject is a mammal.

23. (canceled)

24. A method of labeling or inhibiting nucleolin comprising contacting nucleolin with the compositions of claim 1.

25. The method of claim 24, wherein the nucleolin is contacted by adding the composition to cells comprising nucleolin in vitro.

26. The method of claim 24, wherein the nucleolin is contacted by administering the composition to a subject.


SEQUENCE LISTING (515 sequences)

SEQ ID NO: 1 - RNA, 39 nt, synthetic construct
  misc_feature 1..39: note = Synthetic
  source 1..39: mol_type = other RNA
ccatctagatctccgtagattcccccggctctttctcgc

SEQ ID NO: 2 - RNA, 41 nt, synthetic construct
  misc_feature 1..41: note = Synthetic
  source 1..41: mol_type = other RNA
agccagctttgcataccacgtgcaattcactccacccgtca

SEQ ID NO: 3 - RNA, 40 nt, synthetic construct
  misc_feature 1..40: note = Synthetic
  source 1..40: mol_type = other RNA
aagatctgctaagtgcacgcacaatcaccatcgagcgtct

SEQ ID NO: 4 - RNA, 38 nt, synthetic construct
  misc_feature 1..38: note = Synthetic
  source 1..38: mol_type = other RNA
cacatggtacgcccaaagcgaggcccgctgcgtagtgc

SEQ ID NO: 5 - RNA, 40 nt, synthetic construct
  misc_feature 1..40: note = Synthetic
  source 1..40: mol_type = other RNA
cacggtccagcgctaactgtacctgctgtgccacccaccg

SEQ ID NO: 6 - RNA, 40 nt, synthetic construct
  misc_feature 1..40: note = Synthetic
  source 1..40: mol_type = other RNA
accacgcgccaacgtgtcagctacacgccgtgttccccgg

SEQ ID NO: 7 - RNA, 95 nt, synthetic construct
  misc_feature 1..95: note = Synthetic
  source 1..95: mol_type = other RNA
gggagagaggaagagggatgggccatctagatctccgtagattcccccggctctttctcgccataacccagaggtcgatagtactggatcccccc

SEQ ID NO: 8 - RNA, 97 nt, synthetic construct
  misc_feature 1..97: note = Synthetic
  source 1..97: mol_type = other RNA
gggagagaggaagagggatgggagccagctttgcataccacgtgcaattcactccacccgtcacataacccagaggtcgatagtactggatcccccc

SEQ ID NO: 9 - RNA, 96 nt, synthetic construct
  misc_feature 1..96: note = Synthetic
  source 1..96: mol_type = other RNA
gggagagaggaagagggatgggaagatctgctaagtgcacgcacaatcaccatcgagcgtctcataacccagaggtcgatagtactggatcccccc

SEQ ID NO: 10 - RNA, 94 nt, synthetic construct
  misc_feature 1..94: note = Synthetic
  source 1..94: mol_type = other RNA
gggagagaggaagagggatgggcacatggtacgcccaaagcgaggcccgctgcgtagtgccataacccagaggtcgatagtactggatcccccc

SEQ ID NO: 11 - RNA, 96 nt, synthetic construct
  misc_feature 1..96: note = Synthetic
  source 1..96: mol_type = other RNA
gggagagaggaagagggatgggcacggtccagcgctaactgtacctgctgtgccacccaccgcataacccagaggtcgatagtactggatcccccc

SEQ ID NO: 12 - RNA, 96 nt, synthetic construct
  misc_feature 1..96: note = Synthetic
  source 1..96: mol_type = other RNA
gggagagaggaagagggatgggaccacgcgccaacgtgtcagctacacgccgtgttccccggcataacccagaggtcgatagtactggatcccccc

SEQ ID NO: 13 - RNA, 39 nt, synthetic construct; identical to SEQ ID NO: 1
  misc_feature 1..39: note = Synthetic
  source 1..39: mol_type = other RNA

SEQ ID NO: 14 - RNA, 41 nt, synthetic construct; identical to SEQ ID NO: 2
  misc_feature 1..41: note = Synthetic
  source 1..41: mol_type = other RNA

SEQ ID NO: 15 - RNA, 40 nt, synthetic construct; identical to SEQ ID NO: 3
  misc_feature 1..40: note = Synthetic
  source 1..40: mol_type = other RNA

SEQ ID NO: 16 - RNA, 38 nt, synthetic construct; identical to SEQ ID NO: 4
  misc_feature 1..38: note = Synthetic
  source 1..38: mol_type = other RNA

SEQ ID NO: 17 - RNA, 40 nt, synthetic construct; identical to SEQ ID NO: 5
  misc_feature 1..40: note = Synthetic
  source 1..40: mol_type = other RNA

SEQ ID NO: 18 - RNA, 40 nt, synthetic construct; identical to SEQ ID NO: 6
  misc_feature 1..40: note = Synthetic
  source 1..40: mol_type = other RNA

SEQ ID NO: 19 - RNA, 39 nt, synthetic construct
  misc_feature 1..39: note = Synthetic
  source 1..39: mol_type = other RNA
aagatcctcgcgcatctgccgagcaatcaccatcggacg

SEQ ID NO: 20 - RNA, 40 nt, synthetic construct
  misc_feature 1..40: note = Synthetic
  source 1..40: mol_type = other RNA
ccaaatgccaagccgtagcccggccagtagcccacacgtc

SEQ ID NO: 21 - RNA, 40 nt, synthetic construct
  misc_feature 1..40: note = Synthetic
  source 1..40: mol_type = other RNA
tgccaagccgaggcccggccaccatccactgatagtgggc

SEQ ID NO: 22 - RNA, 40 nt, synthetic construct
  misc_feature 1..40: note = Synthetic
  source 1..40: mol_type = other RNA
aagatcctgacgcgacacagcaatcaccatcgaaccagct

SEQ ID NO: 23 - RNA, 40 nt, synthetic construct
  misc_feature 1..40: note = Synthetic
  source 1..40: mol_type = other RNA
aagatctgcggcaacgcacaatcaccatcgattccgaatt

SEQ ID NO: 24 - RNA, 39 nt, synthetic construct
  misc_feature 1..39: note = Synthetic
  source 1..39: mol_type = other RNA
gagctctcgatttcctccgcgacacccatccaaacctca

SEQ ID NO: 25 - RNA, 40 nt, synthetic construct
  misc_feature 1..40: note = Synthetic
  source 1..40: mol_type = other RNA
ctctccggtctaccatccggaccggcgacaaagtcaactt

SEQ ID NO: 26 - RNA, 41 nt, synthetic construct
  misc_feature 1..41: note = Synthetic
  source 1..41: mol_type = other RNA
aagatctgctatgcacaatcaccatcgggcgctccggggaa

SEQ ID NO: 27 - RNA, 40 nt, synthetic construct
  misc_feature 1..40: note = Synthetic
  source 1..40: mol_type = other RNA
ttgactctgctgcgtagttcgcaccaagatcaaccacttc

SEQ ID NO: 28 - RNA, 38 nt, synthetic construct
  misc_feature 1..38: note = Synthetic
  source 1..38: mol_type = other RNA
taccaagtcgtggcccgactacccagcacgatgcgcaa

SEQ ID NO: 29 - RNA, 39 nt, synthetic construct
  misc_feature 1..39: note = Synthetic
  source 1..39: mol_type = other RNA
ctattcgagttcccacgaatccccccatcgagaacctac

SEQ ID NO: 30 - RNA, 40 nt, synthetic construct
  misc_feature 1..40: note = Synthetic
  source 1..40: mol_type = other RNA
tgccaagccgaggcccggccaccgtccccgcggctgatga

SEQ ID NO: 31 - RNA, 40 nt, synthetic construct
  misc_feature 1..40: note = Synthetic
  source 1..40: mol_type = other RNA
aatgatctcgccaatgggcgacaatcaccatgtcttcaca

SEQ ID NO: 32 - RNA, 38 nt, synthetic construct
  misc_feature 1..38: note = Synthetic
  source 1..38: mol_type = other RNA
tcagtgcgccaagtggaggccccaccgcagcccatcaa

SEQ ID NO: 33 - RNA, 40 nt, synthetic construct
  misc_feature 1..40: note = Synthetic
  source 1..40: mol_type = other RNA
tgtatgccagctttgacgataactgtcgcgcgtcaattca

SEQ ID NO: 34 - RNA, 40 nt, synthetic construct
  misc_feature 1..40: note = Synthetic
  source 1..40: mol_type = other RNA
tacgccaaagtggagcccactcgtaccccatcatgagctg

SEQ ID NO: 35 - RNA, 40 nt, synthetic construct
  misc_feature 1..40: note = Synthetic
  source 1..40: mol_type = other RNA
ccgccagctttgggtaccctgaccaattcacggccatcca

SEQ ID NO: 36 - RNA, 40 nt, synthetic construct
  misc_feature 1..40: note = Synthetic
  source 1..40: mol_type = other RNA
gtaattgtctgagaccaccggacaatcaacaagaaatcct

SEQ ID NO: 37 - RNA, 40 nt, synthetic construct
  misc_feature 1..40: note = Synthetic
  source 1..40: mol_type = other RNA
tcaggccaaagtgtgatagccacacccgcacccatcagga

SEQ ID NO: 38 - RNA, 40 nt, synthetic construct
  misc_feature 1..40: note = Synthetic
  source 1..40: mol_type = other RNA
ccgaccgccgaccagggtgccactcgtacccctgtccgcc

SEQ ID NO: 39 - RNA, 40 nt, synthetic construct
  misc_feature 1..40: note = Synthetic
  source 1..40: mol_type = other RNA
tgccaagtcgaagcccgaccacgccatccctaacagtgcc

SEQ ID NO: 40 - RNA, 40 nt, synthetic construct
  misc_feature 1..40: note = Synthetic
  source 1..40: mol_type = other RNA
acttgtgctgagtcgccaaagtgaggcccactcgccagca

SEQ ID NO: 41 - RNA, 40 nt, synthetic construct
  misc_feature 1..40: note = Synthetic
  source 1..40: mol_type = other RNA
ccgccagctcctctgaggcacaagaggttcacggtgatcc

SEQ ID NO: 42 - RNA, 40 nt, synthetic construct
  misc_feature 1..40: note = Synthetic
  source 1..40: mol_type = other RNA
caccaggttctgctgtccccaagcgctgacccatccttcc

SEQ ID NO: 43 - RNA, 40 nt, synthetic construct
  misc_feature 1..40: note = Synthetic
  source 1..40: mol_type = other RNA
aagatccggtaactccccaccgcaatcaccgtcgactact

SEQ ID NO: 44 - RNA, 40 nt, synthetic construct
  misc_feature 1..40: note = Synthetic
  source 1..40: mol_type = other RNA
ccatctagatctccgtagattccccccggctctttctcgc

SEQ ID NO: 45 - RNA, 40 nt, synthetic construct
  misc_feature 1..40: note = Synthetic
  source 1..40: mol_type = other RNA
ccatctgaacccacagattcccccatcatcagccacagtg

SEQ ID NO: 46 - RNA, 40 nt, synthetic construct
  misc_feature 1..40: note = Synthetic
  source 1..40: mol_type = other RNA
cactaagttggtagccccaactgccccgacacgaggatgt

SEQ ID NO: 47 - RNA, 40 nt, synthetic construct
  misc_feature 1..40: note = Synthetic
  source 1..40: mol_type = other RNA
ttgtgctccgtggctccccggaccaaccgcttccagcagt

SEQ ID NO: 48 - RNA, 39 nt, synthetic construct
  misc_feature 1..39: note = Synthetic
  source 1..39: mol_type = other RNA
caatcacgcgtagtacgtcgcggaagatccccatgccga

SEQ ID NO: 49 - RNA, 39 nt, synthetic construct
  misc_feature 1..39: note = Synthetic
  source 1..39: mol_type = other RNA
cacatggtacgcccaaaagcgaggcccgctgcgtagtgc

SEQ ID NO: 50 - RNA, 40 nt, synthetic construct
  misc_feature 1..40: note = Synthetic
  source 1..40: mol_type = other RNA
tgccatacgcggttcgaagtcgaagcccgacaacccggca

SEQ ID NO: 51 - RNA, 40 nt, synthetic construct
  misc_feature 1..40: note = Synthetic
  source 1..40: mol_type = other RNA
gttattcacatgcctcccgtgaatcaacaagaattccttg

SEQ ID NO: 52 - RNA, 40 nt, synthetic construct
  misc_feature 1..40: note = Synthetic
  source 1..40: mol_type = other RNA
aaagatctagactgtaagtctccaatcgcccagttaattc

SEQ ID NO: 53 - RNA, 39 nt, synthetic construct
  misc_feature 1..39: note = Synthetic
  source 1..39: mol_type = other RNA
gcccaatcgccagtggaacgcgctgaaggatctgcaccc

SEQ ID NO: 54 - RNA, 40 nt, synthetic construct
  misc_feature 1..40: note = Synthetic
  source 1..40: mol_type = other RNA
tgcaacgtaaaagagagtcatctcaggctagtcgtctacc

SEQ ID NO: 55 - RNA, 40 nt, synthetic construct
  misc_feature 1..40: note = Synthetic
  source 1..40: mol_type = other RNA
gtgtacgccaagtcgaggcccgaccgtacccatacgcgac

SEQ ID NO: 56 - RNA, 40 nt, synthetic construct
  misc_feature 1..40: note = Synthetic
  source 1..40: mol_type = other RNA
ttagctctactttcctcttcagtaagactaaccgcttctt

SEQ ID NO: 57 - RNA, 40 nt, synthetic construct
  misc_feature 1..40: note = Synthetic
  source 1..40: mol_type = other RNA
tccaagcggaggccccgcacccaccctccaacgggcacgg

SEQ ID NO: 58 - RNA, 40 nt, synthetic construct
  misc_feature 1..40: note = Synthetic
  source 1..40: mol_type = other RNA
tatcgctccacaacgactcccgtggactacccaattccaa

SEQ ID NO: 59 - RNA, 40 nt, synthetic construct
  misc_feature 1..40: note = Synthetic
  source 1..40: mol_type = other RNA
gtcgtgcccaagtgaaggcctcacgcacgcatcctaacct

SEQ ID NO: 60 - RNA, 40 nt, synthetic construct
  misc_feature 1..40: note = Synthetic
  source 1..40: mol_type = other RNA
aagatctgcgccagcacaatcaccatcgtcctgagaatgg

SEQ ID NO: 61 - RNA, 40 nt, synthetic construct
  misc_feature 1..40: note = Synthetic
  source 1..40: mol_type = other RNA
atgccaagcagtggccctgccacccacctatcactgtcga

SEQ ID NO: 62 - RNA, 40 nt, synthetic construct
  misc_feature 1..40: note = Synthetic
  source 1..40: mol_type = other RNA
aacagaccaagcagcggccctgctctgccatcatacgcct

SEQ ID NO: 63 - RNA, 40 nt, synthetic construct
  misc_feature 1..40: note = Synthetic
  source 1..40: mol_type = other RNA
gtcattcgctgacgaatcaacatgaattcctaactgctga

SEQ ID NO: 64 - RNA, 40 nt, synthetic construct
  misc_feature 1..40: note = Synthetic
  source 1..40: mol_type = other RNA
acacgccaagctggtagccccagccgtgcccattacggcc

SEQ ID NO: 65 - RNA, 40 nt, synthetic construct
  misc_feature 1..40: note = Synthetic
  source 1..40: mol_type = other RNA
tagccaagcagcagccctgccaacccatcctacccgggcg

SEQ ID NO: 66 - RNA, 40 nt, synthetic construct
  misc_feature 1..40: note = Synthetic
  source 1..40: mol_type = other RNA
gcccaaggcgaggcccgccgctccatccagacgctgaggg

SEQ ID NO: 67 - RNA, 40 nt, synthetic construct
  misc_feature 1..40: note = Synthetic
  source 1..40: mol_type = other RNA
aagatctcgtcatgctttgacgtcaatcaccattgttccc

SEQ ID NO: 68 - RNA, 39 nt, synthetic construct
  misc_feature 1..39: note = Synthetic
  source 1..39: mol_type = other RNA
atcccccaggatgagcacgttgccatggactggctatcc

SEQ ID NO: 69 - RNA, 40 nt, synthetic construct
  misc_feature 1..40: note = Synthetic
  source 1..40: mol_type = other RNA
ctgttacagtctcgcgtaacccccccatcgatgtcctcga

SEQ ID NO: 70 - RNA, 40 nt, synthetic construct
  misc_feature 1..40: note = Synthetic
  source 1..40: mol_type = other RNA
agccagctttcggcaaaccgaattcactccaccctgctca

SEQ ID NO: 71 - RNA, 40 nt, synthetic construct
  misc_feature 1..40: note = Synthetic
  source 1..40: mol_type = other RNA
cacggtataacctcctcatatacctgctgtgccacccgcg

SEQ ID NO: 72 - RNA, 40 nt, synthetic construct
  misc_feature 1..40: note = Synthetic
  source 1..40: mol_type = other RNA
ccggaagatctgctcgcactagccggagcccaatcacggc

SEQ ID NO: 73 - RNA, 40 nt, synthetic construct
  misc_feature 1..40: note = Synthetic
  source 1..40: mol_type = other RNA
cctgccgaacggctaagtcgcagcccgacccgcggcaggg

SEQ ID NO: 74 - RNA, 40 nt, synthetic construct
  misc_feature 1..40: note = Synthetic
  source 1..40: mol_type = other RNA
ctccgacccgcggacgaagtcaacttccacagtcccacac

SEQ ID NO: 75 - RNA, 40 nt, synthetic construct
  misc_feature 1..40: note = Synthetic
  source 1..40: mol_type = other RNA
acattaggatctgcgtgatggggatcacccgctacatgtc

SEQ ID NO: 76 - RNA, 40 nt, synthetic construct
  misc_feature 1..40: note = Synthetic
  source 1..40: mol_type = other RNA
tctaagatggggaagatctccggagcaccgggcaatcacc

SEQ ID NO: 77 - RNA, 38 nt, synthetic construct
  misc_feature 1..38: note = Synthetic
  source 1..38: mol_type = other RNA
ctattcgagttcccacgaatcccccatcgagaacctac

SEQ ID NO: 78 - RNA, 40 nt, synthetic construct
  misc_feature 1..40: note = Synthetic
  source 1..40: mol_type = other RNA
tgccaagccgaggcccggccagcatccctcacgagagagg

SEQ ID NO: 79 - RNA, 40 nt, synthetic construct
  misc_feature 1..40: note = Synthetic
  source 1..40: mol_type = other RNA
gccaagcacgtagcccgtgcccccacccgcctgtgtgctg

SEQ ID NO: 80 - RNA, 40 nt, synthetic construct
  misc_feature 1..40: note = Synthetic
  source 1..40: mol_type = other RNA
tgccaagcacgaagcccgtgcccccatccagagtgtgaga

SEQ ID NO: 81 - RNA, 42 nt, synthetic construct
  misc_feature 1..42: note = Synthetic
  source 1..42: mol_type = other RNA
agccagcttttgcataccacgtgcaattcactccacccgtca

SEQ ID NO: 82 - RNA, 40 nt, synthetic construct
  misc_feature 1..40: note = Synthetic
  source 1..40: mol_type = other RNA
ctttgtaaacccggcaaacaaaatcaacttccatcatcaa

SEQ ID NO: 83 - RNA, 40 nt, synthetic construct
  misc_feature 1..40: note = Synthetic
  source 1..40: mol_type = other RNA
ccattgtagcgaccacacaattccccatcggacagcatgg

SEQ ID NO: 84 - RNA, 40 nt, synthetic construct
  misc_feature 1..40: note = Synthetic
  source 1..40: mol_type = other RNA
ctctcgccgttcccaggcacgacaaaatcaacttcccgct

SEQ ID NO: 85 - RNA, 40 nt, synthetic construct
  misc_feature 1..40: note = Synthetic
  source 1..40: mol_type = other RNA
aagccaagccgcggcccggccttcccatgtgctactagag

SEQ ID NO: 86 - RNA, 41 nt, synthetic construct
  misc_feature 1..41: note = Synthetic
  source 1..41: mol_type = other RNA
ccaaatgccaaagccgtagcccggccagtagcccacacgtc

SEQ ID NO: 87 - RNA, 40 nt, synthetic construct
  misc_feature 1..40: note = Synthetic
  source 1..40: mol_type = other RNA
ccattacgcgacgtaattcccccatcgtttcctcgttaag

SEQ ID NO: 88 - RNA, 38 nt, synthetic construct
  misc_feature 1..38: note = Synthetic
  source 1..38: mol_type = other RNA
ccatctagatctccgtagattccccggctctttctcgc

SEQ ID NO: 89 - RNA, 40 nt, synthetic construct
  misc_feature 1..40: note = Synthetic
  source 1..40: mol_type = other RNA
actgtctgcatacacggtatgcccaacgccatccaaaccg

SEQ ID NO: 90 - RNA, 40 nt, synthetic construct
  misc_feature 1..40: note = Synthetic
  source 1..40: mol_type = other RNA
acctgcggctattgccagcgccataagaccctccacagta

SEQ ID NO: 91 - RNA, 39 nt, synthetic construct; identical to SEQ ID NO: 1
  misc_feature 1..39: note = Synthetic
  source 1..39: mol_type = other RNA

SEQ ID NO: 92 - RNA, 39 nt, synthetic construct
  misc_feature 1..39: note = Synthetic
  source 1..39: mol_type = other RNA
ccatctagatctccgtagattcccccggctcttcctcgc

SEQ ID NO: 93 - RNA, 39 nt, synthetic construct
  misc_feature 1..39: note = Synthetic
  source 1..39: mol_type = other RNA
ccatctagatctccgtagattcccccagctctttctcgc

SEQ ID NO: 94 - RNA, 41 nt, synthetic construct; identical to SEQ ID NO: 2
  misc_feature 1..41: note = Synthetic
  source 1..41: mol_type = other RNA

SEQ ID NO: 95 - RNA, 41 nt, synthetic construct
  misc_feature 1..41: note = Synthetic
  source 1..41: mol_type = other RNA
agccagctttgcataccacgtgcaattcactccacccgtcg

SEQ ID NO: 96 - RNA, 40 nt, synthetic construct; identical to SEQ ID NO: 3
  misc_feature 1..40: note = Synthetic
  source 1..40: mol_type = other RNA

SEQ ID NO: 97 - RNA, 40 nt, synthetic construct
  misc_feature 1..40: note = Synthetic
  source 1..40: mol_type = other RNA
aagatctgctaagtgcacgcacaatcaccatcgagcgtcc

SEQ ID NO: 98 - RNA, 40 nt, synthetic construct
  misc_feature 1..40: note = Synthetic
  source 1..40: mol_type = other RNA
aagatctgctaagtgcacgcacaatcaccatcgagcgcct

SEQ ID NO: 99 - RNA, 39 nt, synthetic construct
  misc_feature 1..39: note = Synthetic
  source 1..39: mol_type = other RNA
aagatctgctaagtgcacgcacaatcaccatcgagcgtc

SEQ ID NO: 100 - RNA, 40 nt, synthetic construct
  misc_feature 1..40: note = Synthetic
  source 1..40: mol_type = other RNA
aagatctgctaagtgcacgcacaatcaccatcgagcgact

SEQ ID NO: 101 - RNA, 38 nt, synthetic construct; identical to SEQ ID NO: 4
  misc_feature 1..38: note = Synthetic
  source 1..38: mol_type = other RNA

SEQ ID NO: 102 - RNA, 38 nt, synthetic construct
  misc_feature 1..38: note = Synthetic
  source 1..38: mol_type = other RNA
cacacggtacgcccaaagcgaggcccgctgcgtagtgc

SEQ ID NO: 103 - RNA, 40 nt, synthetic construct; identical to SEQ ID NO: 5
  misc_feature 1..40: note = Synthetic
  source 1..40: mol_type = other RNA

SEQ ID NO: 104 - RNA, 40 nt, synthetic construct
  misc_feature 1..40: note = Synthetic
  source 1..40: mol_type = other RNA
cacggtccagcgctaactgtacctgctgtgccacccacca

SEQ ID NO: 105 - RNA, 40 nt, synthetic construct
  misc_feature 1..40: note = Synthetic
  source 1..40: mol_type = other RNA
cacggtccagcgctaactgtacctgctgtgccacccactg

SEQ ID NO: 106 - RNA, 40 nt, synthetic construct
  misc_feature 1..40: note = Synthetic
  source 1..40: mol_type = other RNA
cacggtccagcgctaactgtacctgctgtgccacccacct

SEQ ID NO: 107 - RNA, 40 nt, synthetic construct
  misc_feature 1..40: note = Synthetic
  source 1..40: mol_type = other RNA
cacggtccagcgctaactgtacctgctgtgccacccgccg

SEQ ID NO: 108 - RNA, 40 nt, synthetic construct; identical to SEQ ID NO: 6
  misc_feature 1..40: note = Synthetic
  source 1..40: mol_type = other RNA

SEQ ID NO: 109 - RNA, 40 nt, synthetic construct
  misc_feature 1..40: note = Synthetic
  source 1..40: mol_type = other RNA
accacgcgccaacgtgtcagctacacgccgtgttccccga

SEQ ID NO: 110 - RNA, 39 nt, synthetic construct
  misc_feature 1..39: note = Synthetic
  source 1..39: mol_type = other RNA
accacgcgccaacgtgtcagctacacgccgtgttccccg

SEQ ID NO: 111 - RNA, 39 nt, synthetic construct
  misc_feature 1..39: note = Synthetic
  source 1..39: mol_type = other RNA
ccacgcgccaacgtgtcagctacacgccgtgttccccgg

SEQ ID NO: 112 - RNA, 39 nt, synthetic construct; identical to SEQ ID NO: 19
  misc_feature 1..39: note = Synthetic
  source 1..39: mol_type = other RNA

SEQ ID NO: 113 - RNA, 39 nt, synthetic construct
  misc_feature 1..39: note = Synthetic
  source 1..39: mol_type = other RNA
aagatcctcgcgcatctgccgagcaatcaccatcggacc

SEQ ID NO: 114 - RNA, 39 nt, synthetic construct
  misc_feature 1..39: note = Synthetic
  source 1..39: mol_type = other RNA
aagatcctcgcgcatctgccgagcaatcaccatcggaca

SEQ ID NO: 115 - RNA, 39 nt, synthetic construct
  misc_feature 1..39: note = Synthetic
  source 1..39: mol_type = other RNA
aagatcctcgcgcatctgccgagcaatcaccatcggact

SEQ ID NO: 116 - RNA, 40 nt, synthetic construct
  misc_feature 1..40: note = Synthetic
  source 1..40: mol_type = other RNA
aaagatcctcgcgcatctgccgagcaatcaccatcggacg

SEQ ID NO: 117 - RNA, 39 nt, synthetic construct
  misc_feature 1..39: note = Synthetic
  source 1..39: mol_type = other RNA
aagatcctcgcgcacctgccgagcaatcaccatcggacg

SEQ ID NO: 118 - RNA, 40 nt, synthetic construct; identical to SEQ ID NO: 20
  misc_feature 1..40: note = Synthetic
  source 1..40: mol_type = other RNA

SEQ ID NO: 119 - RNA, 41 nt, synthetic construct
  misc_feature 1..41: note = Synthetic
  source 1..41: mol_type = other RNA
ccaaaatgccaagccgtagcccggccagtagcccacacgtc

SEQ ID NO: 120 - RNA, 40 nt, synthetic construct
  misc_feature 1..40: note = Synthetic
  source 1..40: mol_type = other RNA
ccaaatgccaagccgtagcccggccagtagcccacacgac

SEQ ID NO: 121 - RNA, 40 nt, synthetic construct
  misc_feature 1..40: note = Synthetic
  source 1..40: mol_type = other RNA
ccaaatgccaagccgtagcccggccagtagcccacacgta

SEQ ID NO: 122 - RNA, 40 nt, synthetic construct; identical to SEQ ID NO: 21
  misc_feature 1..40: note = Synthetic
  source 1..40: mol_type = other RNA

SEQ ID NO: 123 - RNA, 40 nt, synthetic construct
  misc_feature 1..40: note = Synthetic
  source 1..40: mol_type = other RNA
tgccaagccgaggcccggccaccatccactgatagtggga

SEQ ID NO: 124 - RNA, 39 nt, synthetic construct
  misc_feature 1..39: note = Synthetic
  source 1..39: mol_type = other RNA
tgccaagccgaggcccggccaccatccactgatagtggg

SEQ ID NO: 125 - RNA, 40 nt, synthetic construct
  misc_feature 1..40: note = Synthetic
  source 1..40: mol_type = other RNA
tgccaagccgaggcccggccaccatccactgatagtgggt

SEQ ID NO: 126 - RNA, 40 nt, synthetic construct; identical to SEQ ID NO: 22
  misc_feature 1..40: note = Synthetic
  source 1..40: mol_type = other RNA

SEQ ID NO: 127 - RNA, 40 nt, synthetic construct
  misc_feature 1..40: note = Synthetic
  source 1..40: mol_type = other RNA
aagatcctgacgcgacacagcaatcaccatcgaaccagcc

SEQ ID NO: 128 - RNA, 40 nt, synthetic construct; identical to SEQ ID NO: 23
  misc_feature 1..40: note = Synthetic
  source 1..40: mol_type = other RNA

SEQ ID NO: 129 - RNA, 40 nt, synthetic construct
  misc_feature 1..40: note = Synthetic
  source 1..40: mol_type = other RNA
aagatctgcggcaacgcacaatcaccatcgattccgaatg

SEQ ID NO: 130 - RNA, 40 nt, synthetic construct
  misc_feature 1..40: note = Synthetic
  source 1..40: mol_type = other RNA
aagatctgcggcaacgcacaatcaccatcgattccgaatc

SEQ ID NO: 131 - RNA, 40 nt, synthetic construct
  misc_feature 1..40: note = Synthetic
  source 1..40: mol_type = other RNA
aagatctgcggcaacgcacaatcaccatcgattccgaact

SEQ ID NO: 132 - RNA, 40 nt, synthetic construct
  misc_feature 1..40: note = Synthetic
  source 1..40: mol_type = other RNA
aagatctgcggcaacgtacaatcaccatcgattccgaatt

SEQ ID NO: 133 - RNA, 39 nt, synthetic construct; identical to SEQ ID NO: 24
  misc_feature 1..39: note = Synthetic
  source 1..39: mol_type = other RNA

SEQ ID NO: 134 - RNA, 38 nt, synthetic construct
  misc_feature 1..38: note = Synthetic
  source 1..38: mol_type = other RNA
agctctcgatttcctccgcgacacccatccaaacctca

SEQ ID NO: 135 - RNA, 39 nt, synthetic construct
  misc_feature 1..39: note = Synthetic
  source 1..39: mol_type = other RNA
gagctctcgatttcctccgcgacacccatccaaacctcg

SEQ ID NO: 136 - RNA, 40 nt, synthetic construct; identical to SEQ ID NO: 25
  misc_feature 1..40: note = Synthetic
  source 1..40: mol_type = other RNA

SEQ ID NO: 137 - RNA, 40 nt, synthetic construct
  misc_feature 1..40: note = Synthetic
  source 1..40: mol_type = other RNA
ctctccggtctaccacccggaccggcgacaaagtcaactt

SEQ ID NO: 138 - RNA, 41 nt, synthetic construct; identical to SEQ ID NO: 26
  misc_feature 1..41: note = Synthetic
  source 1..41: mol_type = other RNA

SEQ ID NO: 139 - RNA, 40 nt, synthetic construct
  misc_feature 1..40: note = Synthetic
  source 1..40: mol_type = other RNA
aagatctgctatgcacaatcaccatcgggcgctccgggaa

SEQ ID NO: 140 - RNA, 41 nt, synthetic construct
  misc_feature 1..41: note = Synthetic
  source 1..41: mol_type = other RNA
aagatctgctacgcacaatcaccatcgggcgctccggggaa

SEQ ID NO: 141 - RNA, 40 nt, synthetic construct; identical to SEQ ID NO: 27
  misc_feature 1..40: note = Synthetic
  source 1..40: mol_type = other RNA

SEQ ID NO: 142 - RNA, 41 nt, synthetic construct
  misc_feature 1..41: note = Synthetic
  source 1..41: mol_type = other RNA
ttgactctgctgcgtagttcgcaccaagatcaaccacttcc

SEQ ID NO: 143 - RNA, 40 nt, synthetic construct
  misc_feature 1..40: note = Synthetic
  source 1..40: mol_type = other RNA
ttgactctgctgcgtagctcgcaccaagatcaaccacttc

SEQ ID NO: 144 - RNA, 40 nt, synthetic construct
  misc_feature 1..40: note = Synthetic
  source 1..40: mol_type = other RNA
ttgactctgctgcgcagttcgcaccaagatcaaccacttc

SEQ ID NO: 145 - RNA, 40 nt, synthetic construct
  misc_feature 1..40: note = Synthetic
  source 1..40: mol_type = other RNA
ttgactctgctgcgtagtccgcaccaagatcaaccacttc

SEQ ID NO: 146 - RNA, 38 nt, synthetic construct; identical to SEQ ID NO: 28
  misc_feature 1..38: note = Synthetic
  source 1..38: mol_type = other RNA

SEQ ID NO: 147 - RNA, 39 nt, synthetic construct
  misc_feature 1..39: note = Synthetic
  source 1..39: mol_type = other RNA
taccaaagtcgtggcccgactacccagcacgatgcgcaa

SEQ ID NO: 148 - RNA, 38 nt, synthetic construct
  misc_feature 1..38: note = Synthetic
  source 1..38: mol_type = other RNA
taccaagtcgtggcccgactacccagcacggtgcgcaa

SEQ ID NO: 149 - RNA, 38 nt, synthetic construct
  misc_feature 1..38: note = Synthetic
  source 1..38: mol_type = other RNA
taccaagtcgtggcccgactacccagcacgatgcgcag

SEQ ID NO: 150 - RNA, 38 nt, synthetic construct
  misc_feature 1..38: note = Synthetic
  source 1..38: mol_type = other RNA
taccaagtcgtggcccgactacccagcacaatgcgcaa

SEQ ID NO: 151 - RNA, 38 nt, synthetic construct
  misc_feature 1..38: note = Synthetic
  source 1..38: mol_type = other RNA
taccaagtcgcggcccgactacccagcacgatgcgcaa

SEQ ID NO: 152 - RNA, 39 nt, synthetic construct; identical to SEQ ID NO: 29
  misc_feature 1..39: note = Synthetic
  source 1..39: mol_type = other RNA

SEQ ID NO: 153 - RNA, 38 nt, synthetic construct
  misc_feature 1..38: note = Synthetic
  source 1..38: mol_type = other RNA
ctattcgagttcccacgaatccccccatcgagaaccta

SEQ ID NO: 154 - RNA, 39 nt, synthetic construct
  misc_feature 1..39: note = Synthetic
  source 1..39: mol_type = other RNA
ctattcgagttcccacgaatccccccatcgagaacctat

SEQ ID NO: 155 - RNA, 39 nt, synthetic construct
  misc_feature 1..39: note = Synthetic
  source 1..39: mol_type = other RNA
ctattcgagttcccacgaatccccccatcgagaacctaa

SEQ ID NO: 156 - RNA, 40 nt, synthetic construct; identical to SEQ ID NO: 30
  misc_feature 1..40: note = Synthetic
  source 1..40: mol_type = other RNA

SEQ ID NO: 157 - RNA, 41 nt, synthetic construct
  misc_feature 1..41: note = Synthetic
  source 1..41: mol_type = other RNA
tgccaaagccgaggcccggccaccgtccccgcggctgatga

SEQ ID NO: 158 - RNA, 41 nt, synthetic construct
  misc_feature 1..41: note = Synthetic
  source 1..41: mol_type = other RNA
tgccaagccgaggcccggccaccgtccccgcggctgatcga

SEQ ID NO: 159 - RNA, 40 nt, synthetic construct
  misc_feature 1..40: note = Synthetic
  source 1..40: mol_type = other RNA
tgccaagccgaggcccggccaccgtccccgcggctgatgg

SEQ ID NO: 160 - RNA, 40 nt, synthetic construct
  misc_feature 1..40: note = Synthetic
  source 1..40: mol_type = other RNA
tgccaagccgaggcccggccaccgtccccgcggctgacga

SEQ ID NO: 161 - RNA, 40 nt, synthetic construct; identical to SEQ ID NO: 31
  misc_feature 1..40: note = Synthetic
  source 1..40: mol_type = other RNA

SEQ ID NO: 162 - RNA, 40 nt, synthetic construct
  misc_feature 1..40: note = Synthetic
  source 1..40: mol_type = other RNA
aacgatctcgccaatgggcgacaatcaccatgtcttcaca

SEQ ID NO: 163 - RNA, 40 nt, synthetic construct
  misc_feature 1..40: note = Synthetic
  source 1..40: mol_type = other RNA
aatgatctcgccaatgggcgacaatcaccatgtcttcacg

SEQ ID NO: 164 - RNA, 40 nt, synthetic construct
  misc_feature 1..40: note = Synthetic
  source 1..40: mol_type = other RNA
aatgatctcgccaatgtgcgacaatcaccatgtcttcaca

SEQ ID NO: 165 - RNA, 38 nt, synthetic construct; identical to SEQ ID NO: 32
  misc_feature 1..38: note = Synthetic
  source 1..38: mol_type = other RNA

SEQ ID NO: 166 - RNA, 38 nt, synthetic construct
  misc_feature 1..38: note = Synthetic
  source 1..38: mol_type = other RNA
tcagtgcgccaagtggaggccccaccgcagcccatcga

SEQ ID NO: 167 - RNA, 38 nt, synthetic construct
  misc_feature 1..38: note = Synthetic
  source 1..38: mol_type = other RNA
tcagtgcgccaagtggaggccccaccgcagcccatcag

SEQ ID NO: 168 - RNA, 40 nt, synthetic construct; identical to SEQ ID NO: 33
  misc_feature 1..40: note = Synthetic
  source 1..40: mol_type = other RNA

SEQ ID NO: 169 - RNA, 40 nt, synthetic construct; identical to SEQ ID NO: 34
  misc_feature 1..40: note = Synthetic
  source 1..40: mol_type = other RNA

SEQ ID NO: 170 - RNA, 41 nt, synthetic construct
  misc_feature 1..41: note = Synthetic
  source 1..41: mol_type = other RNA
tacgccaaagtggagcccactcgtaccccatcatgagcctg

SEQ ID NO: 171 - RNA, 40 nt, synthetic construct
  misc_feature 1..40: note = Synthetic
  source 1..40: mol_type = other RNA
tacgccaaagtggagcccactcgtaccccatcatgagctc

SEQ ID NO: 172 - RNA, 40 nt, synthetic construct
  misc_feature 1..40: note = Synthetic
  source 1..40: mol_type = other RNA
tacgccaaagtggagcccactcgtaccccatcatgggctg

SEQ ID NO: 173 - RNA, 40 nt, synthetic construct
  misc_feature 1..40: note = Synthetic
  source 1..40: mol_type = other RNA
tacgccaaagtggagcccactcgtatcccatcatgagctg

SEQ ID NO: 174 - RNA, 40 nt, synthetic construct
  misc_feature 1..40: note = Synthetic
  source 1..40: mol_type = other RNA
tacgccaaagtggagcccactcgtaccccatcgtgagctg

SEQ ID NO: 175 - RNA, 40 nt, synthetic construct
  misc_feature 1..40: note = Synthetic
  source 1..40: mol_type = other RNA
tacgccaaagtggagcccactcgtactccatcatgagctg

SEQ ID NO: 176 - RNA, 40 nt, synthetic construct
  misc_feature 1..40: note = Synthetic
  source 1..40: mol_type = other RNA
cacgccaaagtggagcccactcgtaccccatcatgagctg

SEQ ID NO: 177 - RNA, 40 nt, synthetic construct
  misc_feature 1..40: note = Synthetic
  source 1..40: mol_type = other RNA
tacgccaaagtggagcccactcgcaccccatcatgagctg

SEQ ID NO: 178 - RNA, 40 nt, synthetic construct
  misc_feature 1..40: note = Synthetic
  source 1..40: mol_type = other RNA
tacgccaaagtggagcccactcgtaccccatcatgagcta

SEQ ID NO: 179 - RNA, 40 nt, synthetic construct; identical to SEQ ID NO: 35
  misc_feature 1..40: note = Synthetic
  source 1..40: mol_type = other RNA

SEQ ID NO: 180 - RNA, 40 nt, synthetic construct
  misc_feature 1..40: note = Synthetic
  source 1..40: mol_type = other RNA
ccgccagctttgggtaccctgaccaattcacggccatccg

SEQ ID NO: 181 - RNA, 41 nt, synthetic construct
  misc_feature 1..41: note = Synthetic
  source 1..41: mol_type = other RNA
ccgcccagctttgggtaccctgaccaattcacggccatcca

SEQ ID NO: 182 - RNA, 40 nt, synthetic construct; identical to SEQ ID NO: 36
  misc_feature 1..40: note = Synthetic
  source 1..40: mol_type = other RNA

SEQ ID NO: 183 - RNA, 41 nt, synthetic construct
  misc_feature 1..41: note = Synthetic
  source 1..41: mol_type = other RNA
gtaattgtctgagaccaccggacaatcaacaagaaaatcct

SEQ ID NO: 184 - RNA, 39 nt, synthetic construct
  misc_feature 1..39: note = Synthetic
  source 1..39: mol_type = other RNA
taattgtctgagaccaccggacaatcaacaagaaatcct

SEQ ID NO: 185 - RNA, 40 nt, synthetic construct; identical to SEQ ID NO: 37
  misc_feature 1..40: note = Synthetic
  source 1..40: mol_type = other RNA

SEQ ID NO: 186 - RNA, 39 nt, synthetic construct
  misc_feature 1..39: note = Synthetic
  source 1..39: mol_type = other RNA
tcaggccaaagtgtgatagccacacccgcacccatcaga

SEQ ID NO: 187 - RNA, 39 nt, synthetic construct
  misc_feature 1..39: note = Synthetic
  source 1..39: mol_type = other RNA
tcaggccaaagtgtgatagccacacccgcacccatcagg

SEQ ID NO: 188 - RNA, 40 nt, synthetic construct; identical to SEQ ID NO: 38
  misc_feature 1..40: note = Synthetic
  source 1..40: mol_type = other RNA

SEQ ID NO: 189 - RNA, 41 nt, synthetic construct
  misc_feature 1..41: note = Synthetic
  source 1..41: mol_type = other RNA
ccgaccgccgaccagggtgccactcgtacccctgtccgccc

SEQ ID NO: 190 - RNA, 41 nt, synthetic construct
  misc_feature 1..41: note = Synthetic
  source 1..41: mol_type = other RNA
ccgaccgccgaccagggtgccactcgtacccctgtcccgcc

SEQ ID NO: 191 - RNA, 39 nt, synthetic construct
  misc_feature 1..39: note = Synthetic
  source 1..39: mol_type = other RNA
ccgaccgccgaccagggtgccactcgtacccctgtccgc

SEQ ID NO: 192 - RNA, 40 nt, synthetic construct; identical to SEQ ID NO: 39
  misc_feature 1..40: note = Synthetic
  source 1..40: mol_type = other RNA

SEQ ID NO: 193 - RNA, 41 nt, synthetic construct
  misc_feature 1..41: note = Synthetic
  source 1..41: mol_type = other RNA
tgccaaagtcgaagcccgaccacgccatccctaacagtgcc

SEQ ID NO: 194 - RNA, 39 nt, synthetic construct
  misc_feature 1..39: note = Synthetic
  source 1..39: mol_type = other RNA
tgccaagtcgaagcccgaccacgccatccctaacagtgc

SEQ ID NO: 195 - RNA, 40 nt, synthetic construct
  misc_feature 1..40: note = Synthetic
  source 1..40: mol_type = other RNA
tgccaagtcgaagcccgaccacgccatccctaacggtgcc

SEQ ID NO: 196 - RNA, 40 nt, synthetic construct
  misc_feature 1..40: note = Synthetic
  source 1..40: mol_type = other RNA
tgccaagtcgaagcccgaccacgccatccctaacagtgca

SEQ ID NO: 197 - RNA, 40 nt, synthetic construct
  misc_feature 1..40: note = Synthetic
  source 1..40: mol_type = other RNA
tgccaagtcgaggcccgaccacgccatccctaacagtgcc

SEQ ID NO: 198 - RNA, 40 nt, synthetic construct
  misc_feature 1..40: note = Synthetic
  source 1..40: mol_type = other RNA
tgccaagccgaagcccgaccacgccatccctaacagtgcc

SEQ ID NO: 199 - RNA, 40 nt, synthetic construct; identical to SEQ ID NO: 40
  misc_feature 1..40: note = Synthetic
  source 1..40: mol_type = other RNA

SEQ ID NO: 200 - RNA, 40 nt, synthetic construct
  misc_feature 1..40: note = Synthetic
  source 1..40: mol_type = other RNA
gcttgtgctgagtcgccaaagtgaggcccactcgccagca

SEQ ID NO: 201 - RNA, 40 nt, synthetic construct
  misc_feature 1..40: note = Synthetic
  source 1..40: mol_type = other RNA
acctgtgctgagtcgccaaagtgaggcccactcgccagca

SEQ ID NO: 202 - RNA, 40 nt, synthetic construct; identical to SEQ ID NO: 41
  misc_feature 1..40: note = Synthetic
  source 1..40: mol_type = other RNA

SEQ ID NO: 203 - RNA, 41 nt, synthetic construct
  misc_feature 1..41: note = Synthetic
  source 1..41: mol_type = other RNA
ccgccagctcctctgaggcacaagaggttcacggtgatccc

SEQ ID NO: 204 - RNA, 40 nt, synthetic construct; identical to SEQ ID NO: 42
  misc_feature 1..40: note = Synthetic
  source 1..40: mol_type = other RNA

SEQ ID NO: 205 - RNA, 40 nt, synthetic construct
  misc_feature 1..40: note = Synthetic
  source 1..40: mol_type = other RNA
caccaggttctgctatccccaagcgctgacccatccttcc

SEQ ID NO: 206 - RNA, 40 nt, synthetic construct
  misc_feature 1..40: note = Synthetic
  source 1..40: mol_type = other RNA
caccaggttctgctgtctccaagcgctgacccatccttcc

SEQ ID NO: 207 - RNA, 40 nt, synthetic construct
  misc_feature 1..40: note = Synthetic
  source 1..40: mol_type = other RNA
caccaggttctgctgttcccaagcgctgacccatccttcc

SEQ ID NO: 208 - RNA, 40 nt, synthetic construct
  misc_feature 1..40: note = Synthetic
  source 1..40: mol_type = other RNA
caccaggtcctgctgtccccaagcgctgacccatccttcc

SEQ ID NO: 209 - RNA, 40 nt, synthetic construct
  misc_feature 1..40: note = Synthetic
  source 1..40: mol_type = other RNA
caccaggctctgctgtccccaagcgctgacccatccttcc

SEQ ID NO: 210 - RNA, 40 nt, synthetic construct
  misc_feature 1..40: note = Synthetic
  source 1..40: mol_type = other RNA
caccaggttctgctgtcctcaagcgctgacccatccttcc

SEQ ID NO: 211 - RNA, 40 nt, synthetic construct; identical to SEQ ID NO: 43
  misc_feature 1..40: note = Synthetic
  source 1..40: mol_type = other RNA

SEQ ID NO: 212 - RNA, 40 nt, synthetic construct
  misc_feature 1..40: note = Synthetic
  source 1..40: mol_type = other RNA
aagatccggtgactccccaccgcaatcaccgtcgactact

SEQ ID NO: 213 - RNA, 40 nt, synthetic construct
  misc_feature 1..40: note = Synthetic
  source 1..40: mol_type = other RNA
aagatccggtaactccctaccgcaatcaccgtcgactact

SEQ ID NO: 214 - RNA, 41 nt, synthetic construct
  misc_feature 1..41: note = Synthetic
  source 1..41: mol_type = other RNA
aaagatccggtaactccccaccgcaatcaccgtcgactact

SEQ ID NO: 215 - RNA, 40 nt, synthetic construct; identical to SEQ ID NO: 44
  misc_feature 1..40: note = Synthetic
  source 1..40: mol_type = other RNA

SEQ ID NO: 216 - RNA, 40 nt, synthetic construct
  misc_feature 1..40: note = Synthetic
  source 1..40: mol_type = other RNA
ccatctagatctccgtagattcccccgggctctttctcgt

SEQ ID NO: 217 - RNA, 40 nt, synthetic construct
  misc_feature 1..40: note = Synthetic
  source 1..40: mol_type = other RNA
ccatctagatctccgtagattcccccgggctctttctcga

SEQ ID NO: 218 - RNA, 39 nt, synthetic construct
  misc_feature 1..39: note = Synthetic
  source 1..39: mol_type = other RNA
ccatctagatctccgtagattcccccgggctctttctcg

SEQ ID NO: 219 - RNA, 40 nt, synthetic construct
  misc_feature 1..40: note = Synthetic
  source 1..40: mol_type = other RNA
ccatctagatctccgtagattcccccgggctctttctcgc

SEQ ID NO: 220 - RNA, 40 nt, synthetic construct
  misc_feature 1..40: note = Synthetic
  source 1..40: mol_type = other RNA
ccatctagatctccgtagattcccccgggctctttctcac

SEQ ID NO: 221 - RNA, 40 nt, synthetic construct
  misc_feature 1..40: note = Synthetic
  source 1..40: mol_type = other RNA
ccatctagatctccgtagatttcccccggctctttctcgc

SEQ ID NO: 222 - RNA, 40 nt, synthetic construct
  misc_feature 1..40: note = Synthetic
  source 1..40: mol_type = other RNA
ccatctagatctccgtagattcccccgggctcttcctcgc

SEQ ID NO: 223 - RNA, 40 nt, synthetic construct
  misc_feature 1..40: note = Synthetic
  source 1..40: mol_type = other RNA
ccatctagatctccgtagattcccccgggctctctctcgc

SEQ ID NO: 224 - RNA, 40 nt, synthetic construct
  misc_feature 1..40: note = Synthetic
  source 1..40: mol_type = other RNA
ccatctagatctccgtagattcccccgggctctttcttgc

SEQ ID NO: 225 - RNA, 40 nt, synthetic construct
  misc_feature 1..40: note = Synthetic
  source 1..40: mol_type = other RNA
ccatctagatctccgtagattcccccgggccctttctcgc

SEQ ID NO: 226 - RNA, 40 nt, synthetic construct
  misc_feature 1..40: note = Synthetic
  source 1..40: mol_type = other RNA
ccatctagatctccgtagattcccccgggctctttctctc

SEQ ID NO: 227 - RNA, 40 nt, synthetic construct
  misc_feature 1..40: note = Synthetic
  source 1..40: mol_type = other RNA
ccatctagatctccgtagattcccccggcctctttctcgc

SEQ ID NO: 228 - RNA, 40 nt, synthetic construct
  misc_feature 1..40: note = Synthetic
  source 1..40: mol_type = other RNA
ccatctagatctccgtagattcccccgggctctttctccc

SEQ ID NO: 229 - RNA, 41 nt, synthetic construct
  misc_feature 1..41: note = Synthetic
  source 1..41: mol_type = other RNA
ccatctagatctccgtagattcccccgggctctttctcgtc

SEQ ID NO: 230 - RNA, 40 nt, synthetic construct; identical to SEQ ID NO: 45
  misc_feature 1..40: note = Synthetic
  source 1..40: mol_type = other RNA

SEQ ID NO: 231 - RNA, 40 nt, synthetic construct
  misc_feature 1..40: note = Synthetic
  source 1..40: mol_type = other RNA
ccatctgaacccacagattcccccatcatcagccacagta

SEQ ID NO: 232 - RNA, 40 nt, synthetic construct
  misc_feature 1..40: note = Synthetic
  source 1..40: mol_type = other RNA
ccatctgaacccacagattcccccatcatcagccacagcg

SEQ ID NO: 233 - RNA, 40 nt, synthetic construct
  misc_feature 1..40: note = Synthetic
  source 1..40: mol_type = other RNA
ccatctgaacccacagattcccccatcatcagccacagtc

SEQ ID NO: 234 - RNA, 40 nt, synthetic construct
  misc_feature 1..40: note = Synthetic
  source 1..40: mol_type = other RNA
ccatctgaacccacagattcccccatcatcagccacggtg

SEQ ID NO: 235 - RNA, 40 nt, synthetic construct; identical to SEQ ID NO: 46
  misc_feature 1..40: note = Synthetic
  source 1..40: mol_type = other RNA

SEQ ID NO: 236 - RNA, 41 nt, synthetic construct
  misc_feature 1..41: note = Synthetic
  source 1..41: mol_type = other RNA
cactaagttggtagccccaactgccccgacacgaggatgtc

SEQ ID NO: 237 - RNA, 40 nt, synthetic construct
  misc_feature 1..40: note = Synthetic
  source 1..40: mol_type = other RNA
cactaagttggtagccccaactgccccgacacgaggatgc

SEQ ID NO: 238 - RNA, 40 nt, synthetic construct; identical to SEQ ID NO: 47
  misc_feature 1..40: note = Synthetic
  source 1..40: mol_type = other RNA

SEQ ID NO: 239 - RNA, 40 nt, synthetic construct
  misc_feature 1..40: note = Synthetic
  source 1..40: mol_type = other RNA
ttgtgttccgtggctccccggaccaaccgcttccagcagt

SEQ ID NO: 240 - RNA, 40 nt, synthetic construct
  misc_feature 1..40: note = Synthetic
  source 1..40: mol_type = other RNA
ttgtgctccgtggctccccggaccaaccgcttccagcagc

SEQ ID NO: 241 - RNA, 40 nt, synthetic construct
  misc_feature 1..40: note = Synthetic
  source 1..40: mol_type = other RNA
ttgcgctccgtggctccccggaccaaccgcttccagcagt

SEQ ID NO: 242 - RNA, 39 nt, synthetic construct; identical to SEQ ID NO: 48
  misc_feature 1..39: note = Synthetic
  source 1..39: mol_type = other RNA

SEQ ID NO: 243 - RNA, 39 nt, synthetic construct
  misc_feature 1..39: note = Synthetic
  source 1..39: mol_type = other RNA
caatcacgcgtagtacgtcgcggaagatccccatgccgg

SEQ ID NO: 244 - RNA, 39 nt, synthetic construct
  misc_feature 1..39: note = Synthetic
  source 1..39: mol_type = other RNA
caatcacgcgtagtacgtcgcggaagatccccatgccaa

SEQ ID NO: 245 - RNA, 39 nt, synthetic construct
  misc_feature 1..39: note = Synthetic
  source 1..39: mol_type = other RNA
caatcacgcgtagtacgtcgcggaagatccccatgccgt

SEQ ID NO: 246 - RNA, 39 nt, synthetic construct
  misc_feature 1..39: note = Synthetic
  source 1..39: mol_type = other RNA
caatcacgcgtagcacgtcgcggaagatccccatgccga

SEQ ID NO: 247 - RNA, 39 nt, synthetic construct
  misc_feature 1..39: note = Synthetic
  source 1..39: mol_type = other RNA
caatcacgcgtagtacgtcgcggaggatccccatgccga

SEQ ID NO: 248 - RNA, 39 nt, synthetic construct; identical to SEQ ID NO: 49
  misc_feature 1..39: note = Synthetic
  source 1..39: mol_type = other RNA

SEQ ID NO: 249 - RNA, 39 nt, synthetic construct
  misc_feature 1..39: note = Synthetic
  source 1..39: mol_type = other RNA
cacatggtacgccccaaagcgaggcccgctgcgtagtgc

SEQ ID NO: 250 - RNA, 39 nt, synthetic construct
  misc_feature 1..39: note = Synthetic
  source 1..39: mol_type = other RNA
cacatggtacgcccaaagccgaggcccgctgcgtagtgc

SEQ ID NO: 251 - RNA, 38 nt, synthetic construct
  misc_feature 1..38: note = Synthetic
  source 1..38: mol_type = other RNA
cacatggtacgcccaaaagcgaggcccgctgcgtagtg

SEQ ID NO: 252 - RNA, 40 nt, synthetic construct; identical to SEQ ID NO: 50
  misc_feature 1..40: note = Synthetic
  source 1..40: mol_type = other RNA

SEQ ID NO: 253 - RNA, 41 nt, synthetic construct
  misc_feature 1..41: note = Synthetic
  source 1..41: mol_type = other RNA
tgccatacgcggttcgaagtcgaagcccgacaaccccggca

SEQ ID NO: 254 - RNA, 40 nt, synthetic construct
  misc_feature 1..40: note = Synthetic
  source 1..40: mol_type = other RNA
tgccatacgcggttcgaagtcgaggcccgacaacccggca

SEQ ID NO: 255 - RNA, 40 nt, synthetic construct; identical to SEQ ID NO: 51
  misc_feature 1..40: note = Synthetic
  source 1..40: mol_type = other RNA

SEQ ID NO: 256 - RNA, 39 nt, synthetic construct
  misc_feature 1..39: note = Synthetic
  source 1..39: mol_type = other RNA
ttattcacatgcctcccgtgaatcaacaagaattccttg

SEQ ID NO: 257 - RNA, 40 nt, synthetic construct
  misc_feature 1..40: note = Synthetic
  source 1..40: mol_type = other RNA
gttattcacatgcctcccgtgaatcaacaagaattcctcg

SEQ ID NO: 258 - RNA, 40 nt, synthetic construct
  misc_feature 1..40: note = Synthetic
  source 1..40: mol_type = other RNA
gttattcacatgcctctcgtgaatcaacaagaattccttg

SEQ ID NO: 259 - skipped

SEQ ID NO: 260 - RNA, 40 nt, synthetic construct; identical to SEQ ID NO: 52
  misc_feature 1..40: note = Synthetic
  source 1..40: mol_type = other RNA

SEQ ID NO: 261 - RNA, 41 nt, synthetic construct
  misc_feature 1..41: note = Synthetic
  source 1..41: mol_type = other RNA
aaaagatctagactgtaagtctccaatcgcccagttaattc

SEQ ID NO: 262 - RNA, 39 nt, synthetic construct
  misc_feature 1..39: note = Synthetic
  source 1..39: mol_type = other RNA
aaagatctagactgtaagtctccaatcgcccagtaattc

SEQ ID NO: 263 - RNA, 39 nt, synthetic construct; identical to SEQ ID NO: 53
  misc_feature 1..39: note = Synthetic
  source 1..39: mol_type = other RNA

SEQ ID NO: 264 - RNA, 38 nt, synthetic construct
  misc_feature 1..38: note = Synthetic
  source 1..38: mol_type = other RNA
gcccaatcgccagtggaacgcgctgaaggatctgcacc

SEQ ID NO: 265 - RNA, 39 nt, synthetic construct
  misc_feature 1..39: note = Synthetic
  source 1..39: mol_type = other RNA
gcccaatcgccagtggaacgcactgaaggatctgcaccc

SEQ ID NO: 266 - RNA, 40 nt, synthetic construct
  misc_feature 1..40: note = Synthetic
  source 1..40: mol_type = other RNA
gcccaatcgccagtggaacgcgctgaaggatctgcacccc

SEQ ID NO: 267 - RNA, 38 nt, synthetic construct
  misc_feature 1..38: note = Synthetic
  source 1..38: mol_type = other RNA
cccaatcgccagtggaacgcgctgaaggatctgcaccc

SEQ ID NO: 268 - RNA, 39 nt, synthetic construct
  misc_feature 1..39: note = Synthetic
  source 1..39: mol_type = other RNA
gcccaatcgccagcggaacgcgctgaaggatctgcaccc

SEQ ID NO: 269 - RNA, 40 nt, synthetic construct; identical to SEQ ID NO: 54
  misc_feature 1..40: note = Synthetic
  source 1..40: mol_type = other RNA

SEQ ID NO: 270 - RNA, 39 nt, synthetic construct
  misc_feature 1..39: note = Synthetic
  source 1..39: mol_type = other RNA
tgcaacgtaaaagagagtcatctcaggctagtcgtctac

SEQ ID NO: 271 - RNA, 40 nt, synthetic construct; identical to SEQ ID NO: 55
  misc_feature 1..40: note = Synthetic
  source 1..40: mol_type = other RNA

SEQ ID NO: 272 - RNA, 39 nt, synthetic construct
  misc_feature 1..39: note = Synthetic
  source 1..39: mol_type = other RNA
tgtacgccaagtcgaggcccgaccgtacccatacgcgac

SEQ ID NO: 273 - RNA, 40 nt, synthetic construct
  misc_feature 1..40: note = Synthetic
  source 1..40: mol_type = other RNA
gtgtacgccaagtcgaggcccgaccgtacccatacgcggc

SEQ ID NO: 274 - RNA, 40 nt, synthetic construct
  misc_feature 1..40: note = Synthetic
  source 1..40: mol_type = other RNA
gtgtacgccaagtcgaggcccgaccgtacccatacgcgat

SEQ ID NO: 275 - RNA, 40 nt, synthetic construct; identical to SEQ ID NO: 56
  misc_feature 1..40: note = Synthetic
  source 1..40: mol_type = other RNA

SEQ ID NO: 276 - RNA, 40 nt, synthetic construct
  misc_feature 1..40: note = Synthetic
  source 1..40: mol_type = other RNA
ttagctctactttcctcttcagtaagactaaccgcttcct

SEQ ID NO: 277 - RNA, 40 nt, synthetic construct
  misc_feature 1..40: note = Synthetic
  source 1..40: mol_type = other RNA
ttagctctactttcctcttcagtaagactaaccgcttctc

SEQ ID NO: 278 - RNA, 40 nt, synthetic construct
  misc_feature 1..40: note = Synthetic
  source 1..40: mol_type = other RNA
ttagctctactttcctcttcagtaagactaaccgctcctt

SEQ ID NO: 279 - RNA, 40 nt, synthetic construct; identical to SEQ ID NO: 57
  misc_feature 1..40: note = Synthetic
  source 1..40: mol_type = other RNA

SEQ ID NO: 280 - RNA, 40 nt, synthetic construct
  misc_feature 1..40: note = Synthetic
  source 1..40: mol_type = other RNA
tccaagcggaggccccgcacccaccctccaacgggcacgc

SEQ ID NO: 281 - RNA, 40 nt, synthetic construct
  misc_feature 1..40: note = Synthetic
  source 1..40: mol_type = other RNA
tccaagcggaggccccgtacccaccctccaacgggcacgg

SEQ ID NO: 282 - RNA, 40 nt, synthetic construct
  misc_feature 1..40: note = Synthetic
  source 1..40: mol_type = other RNA
tccaagcggaggccccgcacccaccccccaacgggcacgg

SEQ ID NO: 283 - RNA, 40 nt, synthetic construct
  misc_feature 1..40: note = Synthetic
  source 1..40: mol_type = other RNA
tccaagcggaggccccgcacccaccctccaacgggcacga

SEQ ID NO: 284 - RNA, 41 nt, synthetic construct
  misc_feature 1..41: note = Synthetic
  source 1..41: mol_type = other RNA
tccaaagcggaggccccgcacccaccctccaacgggcacgg

SEQ ID NO: 285 - RNA, 40 nt, synthetic construct
  misc_feature 1..40: note = Synthetic
  source 1..40: mol_type = other RNA
tccaagcggaggccccgcacccaccctccaacgggcacag

SEQ ID NO: 286 - RNA, 40 nt, synthetic construct; identical to SEQ ID NO: 58
  misc_feature 1..40: note = Synthetic
  source 1..40: mol_type = other RNA

SEQ ID NO: 287 - RNA, 40 nt, synthetic construct
  misc_feature 1..40: note = Synthetic
  source 1..40: mol_type = other RNA
tatcgctccacaacgactcccgtggactacccaattccag

SEQ ID NO: 288 - RNA, 41 nt, synthetic construct
  misc_feature 1..41: note = Synthetic
  source 1..41: mol_type = other RNA
tatcgctccacaacgactcccgtggactacccaattccaaa

SEQ ID NO: 289 - RNA, 40 nt, synthetic construct
  misc_feature 1..40: note = Synthetic
  source 1..40: mol_type = other RNA
tatcgctccacaacgactcccgtggactacccaattccat

SEQ ID NO: 290 - RNA, 40 nt, synthetic construct; identical to SEQ ID NO: 59
  misc_feature 1..40: note = Synthetic
  source 1..40: mol_type = other RNA

SEQ ID NO: 291 - RNA, 39 nt, synthetic construct
  misc_feature 1..39: note = Synthetic
  source 1..39: mol_type = other RNA
tcgtgcccaagtgaaggcctcacgcacgcatcctaacct

SEQ ID NO: 292 - RNA, 40 nt, synthetic construct
  misc_feature 1..40: note = Synthetic
  source 1..40: mol_type = other RNA
gtcgtgcccaagtgaaggcctcacgcacgcatcctaaccc

SEQ ID NO: 293 - RNA, 40 nt, synthetic construct; identical to SEQ ID NO: 60
  misc_feature 1..40: note = Synthetic
  source 1..40: mol_type = other RNA

SEQ ID NO: 294 - RNA, 40 nt, synthetic construct
  misc_feature 1..40: note = Synthetic
  source 1..40: mol_type = other RNA
aagatctgcgccagcacaatcaccatcgtcctgagaatgc

SEQ ID NO: 295 - RNA, 40 nt, synthetic construct
  misc_feature 1..40: note = Synthetic
  source 1..40: mol_type = other RNA
aagatctgcgccagcacaatcaccatcgtcctgagaatga

SEQ ID NO: 296 - RNA, 40 nt, synthetic construct
  misc_feature 1..40: note = Synthetic
  source 1..40: mol_type = other RNA
aagatctgcgccagcacaatcaccatcgtcctgagagtgg

SEQ ID NO: 297 - RNA, 40 nt, synthetic construct
  misc_feature 1..40: note = Synthetic
  source 1..40: mol_type = other RNA
aagatctgcgccagcacaatcaccatcgtcctgggaatgg

SEQ ID NO: 298 - RNA, 40 nt, synthetic construct; identical to SEQ ID NO: 61
  misc_feature 1..40: note = Synthetic
  source 1..40: mol_type = other RNA

SEQ ID NO: 299 - RNA, 40 nt, synthetic construct
  misc_feature 1..40: note = Synthetic
  source 1..40: mol_type = other RNA
atgccaagcagtcggcctgccacccacctatcactgtcga

SEQ ID NO: 300 - RNA, 40 nt, synthetic construct
  misc_feature 1..40: note = Synthetic
  source 1..40: mol_type = other RNA
atgccaagcagtggccctgccacccacctatcactatcga

SEQ ID NO: 301 - RNA, 40 nt, synthetic construct
  misc_feature 1..40: note = Synthetic
  source 1..40: mol_type = other RNA
atgccaagcagtggccctgccacccacctaccactgtcga

SEQ ID NO: 302 - RNA, 40 nt, synthetic construct
  misc_feature 1..40: note = Synthetic
  source 1..40: mol_type = other RNA
atgccaagcagcggccctgccacccacctatcactgtcga

SEQ ID NO: 303 - RNA, 40 nt, synthetic construct; identical to SEQ ID NO: 62
  misc_feature 1..40: note = Synthetic
  source 1..40: mol_type = other RNA

SEQ ID NO: 304 - RNA, 40 nt, synthetic construct
  misc_feature 1..40: note = Synthetic
  source 1..40: mol_type = other RNA
gacagaccaagcagcggccctgctctgccatcatacgcct

SEQ ID NO: 305 - RNA, 40 nt, synthetic construct
  misc_feature 1..40: note = Synthetic
  source 1..40: mol_type = other RNA
aacagaccaagcagtggccctgctctgccatcatacgcct

SEQ ID NO: 306 - RNA, 40 nt, synthetic construct
  misc_feature 1..40: note = Synthetic
  source 1..40: mol_type = other RNA
aacagaccaagcagcggccctgctctgccatcatacgccc

SEQ ID NO: 307 - RNA, 40 nt, synthetic construct
  misc_feature 1..40: note = Synthetic
  source 1..40: mol_type = other RNA
aacagaccaagcagcggccctgctctgccatcatacacct

SEQ ID NO: 308 - RNA, 39 nt, synthetic construct
  misc_feature 1..39: note = Synthetic
  source 1..39: mol_type = other RNA
acagaccaagcagcggccctgctctgccatcatacgcct

SEQ ID NO: 309 - RNA, 41 nt, synthetic construct
  misc_feature 1..41: note = Synthetic
  source 1..41: mol_type = other RNA
aacagaccaagcagcggccctgctctgccatcatacgccct

SEQ ID NO: 310 - RNA, 40 nt, synthetic construct; identical to SEQ ID NO: 63
  misc_feature 1..40: note = Synthetic
  source 1..40: mol_type = other RNA

SEQ ID NO: 311 - RNA, 39 nt, synthetic construct
  misc_feature 1..39: note = Synthetic
  source 1..39: mol_type = other RNA
tcattcgctgacgaatcaacatgaattcctaactgctga

SEQ ID NO: 312 - RNA, 40 nt, synthetic construct
  misc_feature 1..40: note = Synthetic
  source 1..40: mol_type = other RNA
gtcattcgctgacgaatcaacatgaattcctaactgccga

SEQ ID NO: 313 - RNA, 40 nt, synthetic construct
  misc_feature 1..40: note = Synthetic
  source 1..40: mol_type = other RNA
gtcattcgctgacgaatcaacatgaattcctaactgctgg

SEQ ID NO: 314 - RNA, 40 nt, synthetic construct; identical to SEQ ID NO: 64
  misc_feature 1..40: note = Synthetic
  source 1..40: mol_type = other RNA

SEQ ID NO: 315 - RNA, 39 nt, synthetic construct
  misc_feature 1..39: note = Synthetic
  source 1..39: mol_type = other RNA
acacgccaagctggtagccccagccgtgcccattacggc

SEQ ID NO: 316 - RNA, 40 nt, synthetic construct
  misc_feature 1..40: note = Synthetic
  source 1..40: mol_type = other RNA
acacgccaagctggtagccccagccgtgcccattacggtc

SEQ ID NO: 317 - RNA, 40 nt, synthetic construct
  misc_feature 1..40: note = Synthetic
  source 1..40: mol_type = other RNA
acacgccaagctggtagccccagccgtacccattacggcc

SEQ ID NO: 318 - RNA, 40 nt, synthetic construct; identical to SEQ ID NO: 65
  misc_feature 1..40: note = Synthetic
  source 1..40: mol_type = other RNA

SEQ ID NO: 319 - RNA, 39 nt, synthetic construct
  misc_feature 1..39: note = Synthetic
  source 1..39: mol_type = other RNA
tagccaagcagcagccctgccaacccatcctacccggcg

SEQ ID NO: 320 - RNA, 40 nt, synthetic construct
  misc_feature 1..40: note = Synthetic
  source 1..40: mol_type = other RNA
tagccaagcagcagccctgccaacccatcctacccgggca

SEQ ID NO: 321 - RNA, 40 nt, synthetic construct
  misc_feature 1..40: note = Synthetic
  source 1..40: mol_type = other RNA
tagccaagcagcagccctgccaacccatcctacccgggtg

SEQ ID NO: 322 - RNA, 40 nt, synthetic construct
  misc_feature 1..40: note = Synthetic
  source 1..40: mol_type = other RNA
tagccaagcagcggccctgccaacccatcctacccgggcg

SEQ ID NO: 323 - RNA, 40 nt, synthetic construct; identical to SEQ ID NO: 66
  misc_feature 1..40: note = Synthetic
  source 1..40: mol_type = other RNA

SEQ ID NO: 324 - RNA, 39 nt, synthetic construct
  misc_feature 1..39: note = Synthetic
  source 1..39: mol_type = other RNA
gcccaaggcgaggcccgccgctccatccagacgctgagg

SEQ ID NO: 325 - RNA, 39 nt, synthetic construct
  misc_feature 1..39: note = Synthetic
  source 1..39: mol_type = other RNA
cccaaggcgaggcccgccgctccatccagacgctgaggg

SEQ ID NO: 326 - RNA, 38 nt, synthetic construct
  misc_feature 1..38: note = Synthetic
  source 1..38: mol_type = other RNA
cccaaggcgaggcccgccgctccatccagacgctgagg

SEQ ID NO: 327 - RNA, 40 nt, synthetic construct
  misc_feature 1..40: note = Synthetic
  source 1..40: mol_type = other RNA
gcccaaggcgaggcccgccgctccatccagacgctgaggc

SEQ ID NO: 328 - RNA, 41 nt, synthetic construct
  misc_feature 1..41: note = Synthetic
  source 1..41: mol_type = other RNA
gcccaaaggcgaggcccgccgctccatccagacgctgaggg

SEQ ID NO: 329 - RNA, 40 nt, synthetic construct
  misc_feature 1..40: note = Synthetic
  source 1..40: mol_type = other RNA
gcccaaggcgaggcccgccgctccatccagacgctgagga

SEQ ID NO: 330 - RNA, 41 nt, synthetic construct
  misc_feature 1..41: note = Synthetic
  source 1..41: mol_type = other RNA
gccccaaggcgaggcccgccgctccatccagacgctgaggg

SEQ ID NO: 331 - RNA, 40 nt, synthetic construct; identical to SEQ ID NO: 67
  misc_feature 1..40: note = Synthetic
  source 1..40: mol_type = other RNA

SEQ ID NO: 332 - RNA, 39 nt, synthetic construct
  misc_feature 1..39: note = Synthetic
  source 1..39: mol_type = other RNA
aagatctcgtcatgctttgacgtcaatcaccattgttcc

SEQ ID NO: 333 - RNA, 40 nt, synthetic construct
  misc_feature 1..40: note = Synthetic
  source 1..40: mol_type = other RNA
aagatctcgtcatgctttgacgccaatcaccattgttccc

SEQ ID NO: 334 - RNA, 40 nt, synthetic construct
  misc_feature 1..40: note = Synthetic
  source 1..40: mol_type = other RNA
aagatctcgtcatgctttgacgtcaatcaccattgttcca

SEQ ID NO: 335 - RNA, 40 nt, synthetic construct
  misc_feature 1..40: note = Synthetic
  source 1..40: mol_type = other RNA
aagatctcgtcatgctttgacgtcaatcaccattgttcct

SEQ ID NO: 336 - RNA, 41 nt, synthetic construct
  misc_feature 1..41: note = Synthetic
  source 1..41: mol_type = other RNA
aagatctcgtcatgctttgacgtcaatcaccattgttcccc

SEQ ID NO: 337 - RNA, 41 nt, synthetic construct
  misc_feature 1..41: note = Synthetic
  source 1..41: mol_type = other RNA
aaagatctcgtcatgctttgacgtcaatcaccattgttccc

SEQ ID NO: 338 - RNA, 40 nt, synthetic construct
  misc_feature 1..40: note = Synthetic
  source 1..40: mol_type = other RNA
aagatctcgtcatgccttgacgtcaatcaccattgttccc

SEQ ID NO: 339 - RNA, 39 nt, synthetic construct; identical to SEQ ID NO: 68
  misc_feature 1..39: note = Synthetic
  source 1..39: mol_type = other RNA

SEQ ID NO: 340 - RNA, 38 nt, synthetic construct
  misc_feature 1..38: note = Synthetic
  source 1..38: mol_type = other RNA
atccccaggatgagcacgttgccatggactggctatcc

SEQ ID NO: 341 - RNA, 40 nt, synthetic construct; identical to SEQ ID NO: 69
  misc_feature 1..40: note = Synthetic
  source 1..40: mol_type = other RNA

SEQ ID NO: 342 - RNA, 40 nt, synthetic construct
  misc_feature 1..40: note = Synthetic
  source 1..40: mol_type = other RNA
ctgttacagtctcgcgtaacccccccatcgatgtcctcgg

SEQ ID NO: 343 - RNA, 40 nt, synthetic construct
  misc_feature 1..40: note = Synthetic
  source 1..40: mol_type = other RNA
ctgttacagtctcgagtaacccccccatcgatgtcctcga

SEQ ID NO: 344 - RNA, 40 nt, synthetic construct
  misc_feature 1..40: note = Synthetic
  source 1..40: mol_type = other RNA
ctgttacagtctcgcgtaacccctccatcgatgtcctcga

SEQ ID NO: 345 - RNA, 40 nt, synthetic construct
  misc_feature 1..40: note = Synthetic
  source 1..40: mol_type = other RNA
ctgttacagcctcgcgtaacccccccatcgatgtcctcga

SEQ ID NO: 346 - RNA, 40 nt, synthetic construct
  misc_feature 1..40: note = Synthetic
  source 1..40: mol_type = other RNA
ctgttacagtctcccgtaacccccccatcgatgtcctcga

SEQ ID NO: 347 - RNA, 40 nt, synthetic construct; identical to SEQ ID NO: 70
  misc_feature 1..40: note = Synthetic
  source 1..40: mol_type = other RNA

SEQ ID NO: 348 - RNA, 40 nt, synthetic construct
  misc_feature 1..40: note = Synthetic
  source 1..40: mol_type = other RNA
agccagctttcggcaaaccgaattcactccaccctcctca

SEQ ID NO: 349 - RNA, 40 nt, synthetic construct
  misc_feature 1..40: note = Synthetic
  source 1..40: mol_type = other RNA
agccagctttcggcaaaccgaattcactccgccctgctca

SEQ ID NO: 350 - RNA, 38 nt, synthetic construct
  misc_feature 1..38: note = Synthetic
  source 1..38: mol_type = other RNA
agccagctttcggcaaaccgaattcactccaccctgct

SEQ ID NO: 351 - RNA, 40 nt, synthetic construct
  misc_feature 1..40: note = Synthetic
  source 1..40: mol_type = other RNA
agccagctttcggcgaaccgaattcactccaccctgctca

SEQ ID NO: 352 - RNA, 40 nt, synthetic construct
  misc_feature 1..40: note = Synthetic
  source 1..40: mol_type = other RNA
agccagctttcggcaaaccgaattcactccaccctgctcg

SEQ ID NO: 353 - RNA, 39 nt, synthetic construct
  misc_feature 1..39: note = Synthetic
  source 1..39: mol_type = other RNA
agccagctttcggcaaaccgaattcactccaccctgctc

SEQ ID NO: 354 - RNA, 40 nt, synthetic construct
  misc_feature 1..40: note = Synthetic
  source 1..40: mol_type = other RNA
agccagctttcggcaaaccgaattcactccaccctgcaca

SEQ ID NO: 355 - RNA, 40 nt, synthetic construct; identical to SEQ ID NO: 71
  misc_feature 1..40: note = Synthetic
  source 1..40: mol_type = other RNA

SEQ ID NO: 356 - RNA, 40 nt, synthetic construct
  misc_feature 1..40: note = Synthetic
  source 1..40: mol_type = other RNA
cacggtataacctcctcatatacctgctgtgccacccgca

SEQ ID NO: 357 - RNA, 41 nt, synthetic construct
  misc_feature 1..41: note = Synthetic
  source 1..41: mol_type = other RNA
cacggtataacctcctcatatacctgctgtgccacccaccg

SEQ ID NO: 358 - RNA, 40 nt, synthetic construct
  misc_feature 1..40: note = Synthetic
  source 1..40: mol_type = other RNA
cacggtataacctcctcatatacctgctgtgccacccgct

SEQ ID NO: 359 - RNA, 40 nt, synthetic construct
  misc_feature 1..40: note = Synthetic
  source 1..40: mol_type = other RNA
cacggtataacctcctcatatacctgctgtgccacccacg

SEQ ID NO: 360 - RNA, 40 nt, synthetic construct
  misc_feature 1..40: note = Synthetic
  source 1..40: mol_type = other RNA
cacggtataacctcctcatatacctgctgtgccacccgtg

SEQ ID NO: 361 - RNA, 40 nt, synthetic construct
  misc_feature 1..40: note = Synthetic
  source 1..40: mol_type = other RNA
cacggtataacctcctcatatacctgctgtgccgcccgcg

SEQ ID NO: 362 - RNA, 40 nt, synthetic construct; identical to SEQ ID NO: 72
  misc_feature 1..40: note = Synthetic
  source 1..40: mol_type = other RNA

SEQ ID NO: 363 - RNA, 40 nt, synthetic construct
  misc_feature 1..40: note = Synthetic
  source 1..40: mol_type = other RNA
ccggaagatctgctcgcactagtcggagcccaatcacggc

SEQ ID NO: 364 - RNA, 40 nt, synthetic construct
  misc_feature 1..40: note = Synthetic
  source 1..40: mol_type = other RNA
ccggaggatctgctcgcactagccggagcccaatcacggc

SEQ ID NO: 365 - RNA, 40 nt, synthetic construct
  misc_feature 1..40: note = Synthetic
  source 1..40: mol_type = other RNA
ccggaagatctgctcgcattagccggagcccaatcacggc

SEQ ID NO: 366 - RNA, 40 nt, synthetic construct; identical to SEQ ID NO: 73
  misc_feature 1..40: note = Synthetic
  source 1..40: mol_type = other RNA

SEQ ID NO: 367 - RNA, 39 nt, synthetic construct
  misc_feature 1..39: note = Synthetic
  source 1..39: mol_type = other RNA
cctgccgaacggctaagtcgcagcccgacccgcggcagg

SEQ ID NO: 368 - RNA, 40 nt, synthetic construct
  misc_feature 1..40: note = Synthetic
  source 1..40: mol_type = other RNA
cctgccgaacggctaagtcgcagcccgacccgcggcagga

SEQ ID NO: 369 - RNA, 40 nt, synthetic construct
  misc_feature 1..40: note = Synthetic
  source 1..40: mol_type = other RNA
cctgccgaacggccaagtcgcagcccgacccgcggcaggg

SEQ ID NO: 370 - RNA, 40 nt, synthetic construct
  misc_feature 1..40: note = Synthetic
  source 1..40: mol_type = other RNA
cctgccgaacggctaagtcgcggcccgacccgcggcaggg

SEQ ID NO: 371 - RNA, 40 nt, synthetic construct; identical to SEQ ID NO: 74
  misc_feature 1..40: note = Synthetic
  source 1..40: mol_type = other RNA

SEQ ID NO: 372 - RNA, 40 nt, synthetic construct
  misc_feature 1..40: note = Synthetic
  source 1..40: mol_type = other RNA
ctccgacccgcggacgaagtcaacttccacagtcccacaa

SEQ ID NO: 373 - RNA, 42 nt, synthetic construct
  misc_feature 1..42: note = Synthetic
  source 1..42: mol_type = other RNA
ctccgacccgcggacgaagtcaacttccacagtcccacacac

SEQ ID NO: 374 - RNA, 40 nt, synthetic construct
  misc_feature 1..40: note = Synthetic
  source 1..40: mol_type = other RNA
ctccgacccgcggacgaagtcaacttccacagtctcacac

SEQ ID NO: 375 - RNA, 40 nt, synthetic construct
  misc_feature 1..40: note = Synthetic
  source 1..40: mol_type = other RNA
ctccgacccgcggacgaagtcaacttccacagtcccacat

SEQ ID NO: 376 - RNA, 40 nt, synthetic construct
  misc_feature 1..40: note = Synthetic
  source 1..40: mol_type = other RNA
ctccgacccgcggacgaagtcaacttccacagtcccgcac

SEQ ID NO: 377 - RNA, 40 nt, synthetic construct
  misc_feature 1..40: note = Synthetic
  source 1..40: mol_type = other RNA
ctccgacccgcggacgaagtcaacttccacggtcccacac

SEQ ID NO: 378 - RNA, 40 nt, synthetic construct
  misc_feature 1..40: note = Synthetic
  source 1..40: mol_type = other RNA
ctccgacccgcggacgaagtcaacttccacagtcccatac

SEQ ID NO: 379 - RNA, 40 nt, synthetic construct; identical to SEQ ID NO: 75
  misc_feature 1..40: note = Synthetic
  source 1..40: mol_type = other RNA

SEQ ID NO: 380 - RNA, 41 nt, synthetic construct
  misc_feature 1..41: note = Synthetic
  source 1..41: mol_type = other RNA
acatttaggatctgcgtgatggggatcacccgctacatgtc

SEQ ID NO: 381 - RNA, 40 nt, synthetic construct
  misc_feature 1..40: note = Synthetic
  source 1..40: mol_type = other RNA
gcattaggatctgcgtgatggggatcacccgctacatgtc

SEQ ID NO: 382 - RNA, 40 nt, synthetic construct
  misc_feature 1..40: note = Synthetic
  source 1..40: mol_type = other RNA
acattaggatctgcgcgatggggatcacccgctacatgtc

SEQ ID NO: 383 - RNA, 40 nt, synthetic construct; identical to SEQ ID NO: 76
  misc_feature 1..40: note = Synthetic
  source 1..40: mol_type = other RNA

SEQ ID NO: 384 - RNA, 41 nt, synthetic construct
  misc_feature 1..41: note = Synthetic
  source 1..41: mol_type = other RNA
tctaagatggggaagatctccggagcaccgggcaatcaccc

SEQ ID NO: 385 - RNA, 40 nt, synthetic construct
  misc_feature 1..40: note = Synthetic
  source 1..40: mol_type = other RNA
cctaagatggggaagatctccggagcaccgggcaatcacc

SEQ ID NO: 386 - RNA, 40 nt, synthetic construct
  misc_feature 1..40: note = Synthetic
  source 1..40: mol_type = other RNA
tctaaggtggggaagatctccggagcaccgggcaatcacc

SEQ ID NO: 387 - RNA, 40 nt, synthetic construct
  misc_feature 1..40: note = Synthetic
  source 1..40: mol_type = other RNA
tctaagatggggaagatctccggagcgccgggcaatcacc

SEQ ID NO: 388 - RNA, 38 nt, synthetic construct; identical to SEQ ID NO: 77
  misc_feature 1..38: note = Synthetic
  source 1..38: mol_type = other RNA

SEQ ID NO: 389 - RNA, 38 nt, synthetic construct
  misc_feature 1..38: note = Synthetic
  source 1..38: mol_type = other RNA
ctattcgagttcccacgaatccccccatcagaacctac

SEQ ID NO: 390 - RNA, 38 nt, synthetic construct
  misc_feature 1..38: note = Synthetic
  source 1..38: mol_type = other RNA
ctactcgagttcccacgaatcccccatcgagaacctac

SEQ ID NO: 391 - RNA, 38 nt, synthetic construct
  misc_feature 1..38: note = Synthetic
  source 1..38: mol_type = other RNA
ctattcgagttcccacgaatcccccatcaagaacctac

SEQ ID NO: 392 - RNA, 40 nt, synthetic construct; identical to SEQ ID NO: 78
  misc_feature 1..40: note = Synthetic
  source 1..40: mol_type = other RNA

SEQ ID NO: 393 - RNA, 41 nt, synthetic construct
  misc_feature 1..41: note = Synthetic
  source 1..41: mol_type = other RNA
tgccaaagccgaggcccggccagcatccctcacgagagagg

SEQ ID NO: 394 - RNA, 40 nt, synthetic construct
  misc_feature 1..40: note = Synthetic
  source 1..40: mol_type = other RNA
tgccaagccgaggcccggccagcatccctcacgagagagc

SEQ ID NO: 395 - RNA, 39 nt, synthetic construct
  misc_feature 1..39: note = Synthetic
  source 1..39: mol_type = other RNA
tgccaagccgaggcccggccagcatccctcacgagagag

SEQ ID NO: 396 - RNA, 40 nt, synthetic construct
  misc_feature 1..40: note = Synthetic
  source 1..40: mol_type = other RNA
tgccaagccgaggcccggccagcatcccccacgagagagg

SEQ ID NO: 397 - RNA, 40 nt, synthetic construct
  misc_feature 1..40: note = Synthetic
  source 1..40: mol_type = other RNA
tgccaagccgaggcccggccagcatccctcacgagagaga

SEQ ID NO: 398 - RNA, 40 nt, synthetic construct
  misc_feature 1..40: note = Synthetic
  source 1..40: mol_type = other RNA
tgccaagccggggcccggccagcatccctcacgagagagg

SEQ ID NO: 399 - RNA, 39 nt, synthetic construct
  misc_feature 1..39: note = Synthetic
  source 1..39: mol_type = other RNA
tgccaagccgaggcccggccagcatccctcacgagaggg

SEQ ID NO: 400 - RNA, 40 nt, synthetic construct; identical to SEQ ID NO: 79
  misc_feature 1..40: note = Synthetic
  source 1..40: mol_type = other RNA

SEQ ID NO: 401 - RNA, 39 nt, synthetic construct
  misc_feature 1..39: note = Synthetic
  source 1..39: mol_type = other RNA
ccaagcacgtagcccgtgcccccacccgcctgtgtgctg

SEQ ID NO: 402 - RNA, 40 nt, synthetic construct
  misc_feature 1..40: note = Synthetic
  source 1..40: mol_type = other RNA
gccaagcacgtagcccgtgcccccacccgcctgtgtgcgg

SEQ ID NO: 403 - RNA, 40 nt, synthetic construct
  misc_feature 1..40: note = Synthetic
  source 1..40: mol_type = other RNA
gccaagcacgtagcccgtgcccccacccacctgtgtgctg

SEQ ID NO: 404 - RNA, 40 nt, synthetic construct
  misc_feature 1..40: note = Synthetic
  source 1..40: mol_type = other RNA
gccaagcacgtagcccgtgcccccacccgcctgtgtgctc

SEQ ID NO: 405 - RNA, 40 nt, synthetic construct
  misc_feature 1..40: note = Synthetic
  source 1..40: mol_type = other RNA
gccaagcacgtagcccgtgcccccacccgcctgtgtgccg

SEQ ID NO: 406 - RNA, 41 nt, synthetic construct
  misc_feature 1..41: note = Synthetic
  source 1..41: mol_type = other RNA
gccaaagcacgtagcccgtgcccccacccgcctgtgtgctg

SEQ ID NO: 407 - RNA, 40 nt, synthetic construct
  misc_feature 1..40: note = Synthetic
  source 1..40: mol_type = other RNA
gccaagcacgtagcccgtgcccccacccgcctgtgtgcta

SEQ ID NO: 408 - RNA, 40 nt, synthetic construct; identical to SEQ ID NO: 80
  misc_feature 1..40: note = Synthetic
  source 1..40: mol_type = other RNA

SEQ ID NO: 409 - RNA, 41 nt, synthetic construct
  misc_feature 1..41: note = Synthetic
  source 1..41: mol_type = other RNA
tgccaaagcacgaagcccgtgcccccatccagagtgtgaga

SEQ ID NO: 410 - RNA, 40 nt, synthetic construct
  misc_feature 1..40: note = Synthetic
  source 1..40: mol_type = other RNA
tgccaagcacgaagcccgtgcccccatccagagtgtggga

SEQ ID NO: 411 - RNA, 40 nt, synthetic construct
  misc_feature 1..40: note = Synthetic
  source 1..40: mol_type = other RNA
tgccaagcacgaggcccgtgcccccatccagagtgtgaga

SEQ ID NO: 412 - RNA, 40 nt, synthetic construct
  misc_feature 1..40: note = Synthetic
  source 1..40: mol_type = other RNA
tgccaagcacgaagcccgtgcccccattcagagtgtgaga

SEQ ID NO: 413 - RNA, 40 nt, synthetic construct
  misc_feature 1..40: note = Synthetic
  source 1..40: mol_type = other RNA
tgccaagcacgaagcccgtgcccccatccagagtgcgaga

SEQ ID NO: 414 - RNA, 40 nt, synthetic construct
  misc_feature 1..40: note = Synthetic
  source 1..40: mol_type = other RNA
tgccaagcacgaagcccgtgcccccatccagagcgtgaga

SEQ ID NO: 415 - RNA, 40 nt, synthetic construct
  misc_feature 1..40: note = Synthetic
  source 1..40: mol_type = other RNA
tgccaagcacgaagcccgtgcccccatccagagtgtgagg

SEQ ID NO: 416 - RNA, 40 nt, synthetic construct
  misc_feature 1..40: note = Synthetic
  source 1..40: mol_type = other RNA
tgccaagcacgaagcccgtgcccccatccagggtgtgaga

SEQ ID NO: 417 - RNA, 42 nt, synthetic construct; identical to SEQ ID NO: 81
  misc_feature 1..42: note = Synthetic
  source 1..42: mol_type = other RNA

SEQ ID NO: 418 - RNA, 42 nt, synthetic construct
  misc_feature 1..42: note = Synthetic
  source 1..42: mol_type = other RNA
agccagctttgccataccacgtgcaattcactccacccgtca

SEQ ID NO: 419 - RNA, 42 nt, synthetic construct
  misc_feature 1..42: note = Synthetic
  source 1..42: mol_type = other RNA
agccagcctttgcataccacgtgcaattcactccacccgtca

SEQ ID NO: 420 - RNA, 42 nt, synthetic construct
  misc_feature 1..42: note = Synthetic
  source 1..42: mol_type = other RNA
agccagcttttgcataccacgtgcaattcactccacccgtcg

SEQ ID NO: 421 - RNA, 42 nt, synthetic construct
  misc_feature 1..42: note = Synthetic
  source 1..42: mol_type = other RNA
agccagcttttgcacaccacgtgcaattcactccacccgtca

SEQ ID NO: 422 - RNA, 42 nt, synthetic construct
  misc_feature 1..42: note = Synthetic
  source 1..42: mol_type = other RNA
agccaagctttgcataccacgtgcaattcactccacccgtca

SEQ ID NO: 423 - RNA, 40 nt, synthetic construct; identical to SEQ ID NO: 82
  misc_feature 1..40: note = Synthetic
  source 1..40: mol_type = other RNA

SEQ ID NO: 424 - RNA, 40 nt, synthetic construct
  misc_feature 1..40: note = Synthetic
  source 1..40: mol_type = other RNA
ctttgtaaacccggcaaacaaaatcaacttccatcaccaa

SEQ ID NO: 425 - RNA, 40 nt, synthetic construct; identical to SEQ ID NO: 83
  misc_feature 1..40: note = Synthetic
  source 1..40: mol_type = other RNA

SEQ ID NO: 426 - RNA, 39 nt, synthetic construct
  misc_feature 1..39: note = Synthetic
  source 1..39: mol_type = other RNA
ccattgtagcgaccacacaattccccatcggacagcatg

SEQ ID NO: 427 - RNA, 40 nt, synthetic construct
  misc_feature 1..40: note = Synthetic
  source 1..40: mol_type = other RNA
ccattgtagcgaccacacaattccccatcggacagcgtgg

SEQ ID NO: 428 - RNA, 40 nt, synthetic construct
  misc_feature 1..40: note = Synthetic
  source 1..40: mol_type = other RNA
ccattgtagcgaccacacaattccccatcggacagcacgg

SEQ ID NO: 429 - RNA, 40 nt, synthetic construct
  misc_feature 1..40: note = Synthetic
  source 1..40: mol_type = other RNA
ccattgtagcgaccacacaattccccatcggacagcatgc

SEQ ID NO: 430 - RNA, 40 nt, synthetic construct
  misc_feature 1..40: note = Synthetic
  source 1..40: mol_type = other RNA
ccattgtagcgaccacacaatcccccatcggacagcatgg

SEQ ID NO: 431 - RNA, 40 nt, synthetic construct
  misc_feature 1..40: note = Synthetic
  source 1..40: mol_type = other RNA
ccattgtagcgaccacacaattccccatcggacagcatgt

SEQ ID NO: 432 - RNA, 40 nt, synthetic construct; identical to SEQ ID NO: 84
  misc_feature 1..40: note = Synthetic
  source 1..40: mol_type = other RNA

SEQ ID NO: 433 - RNA, 40 nt, synthetic construct
  misc_feature 1..40: note = Synthetic
  source 1..40: mol_type = other RNA
ctctcgccgttcccaggcgcgacaaaatcaacttcccgct

SEQ ID NO: 434 - RNA, 40 nt, synthetic construct
  misc_feature 1..40: note = Synthetic
  source 1..40: mol_type = other RNA
ctctcgccgttcccgggcacgacaaaatcaacttcccgct

SEQ ID NO: 435 - RNA, 40 nt, synthetic construct
  misc_feature 1..40: note = Synthetic
  source 1..40: mol_type = other RNA
ctctcgccgttcccaggcacgacaaaatcaacttcccgca

SEQ ID NO: 436 - RNA, 40 nt, synthetic construct; identical to SEQ ID NO: 85
  misc_feature 1..40: note = Synthetic
  source 1..40: mol_type = other RNA

SEQ ID NO: 437 - RNA, 41 nt, synthetic construct
  misc_feature 1..41: note = Synthetic
  source 1..41: mol_type = other RNA
aaagccaagccgcggcccggccttcccatgtgctactagag

SEQ ID NO: 438 - RNA, 41 nt, synthetic construct
  misc_feature 1..41: note = Synthetic
  source 1..41: mol_type = other RNA
aagccaaagccgcggcccggccttcccatgtgctactagag

SEQ ID NO: 439 - RNA, 40 nt, synthetic construct
  misc_feature 1..40: note = Synthetic
  source 1..40: mol_type = other RNA
gagccaagccgcggcccggccttcccatgtgctactagag

SEQ ID NO: 440 - RNA, 39 nt, synthetic construct
  misc_feature 1..39: note = Synthetic
  source 1..39: mol_type = other RNA
agccaagccgcggcccggccttcccatgtgctactagag

SEQ ID NO: 441 - RNA, 40 nt, synthetic construct
  misc_feature 1..40: note = Synthetic
  source 1..40: mol_type = other RNA
aagccaagccgtggcccggccttcccatgtgctactagag

SEQ ID NO: 442 - RNA, 39 nt, synthetic construct
  misc_feature 1..39: note = Synthetic
  source 1..39: mol_type = other RNA
tgccaagccgcggcccggccttcccatgtgctactagag

SEQ ID NO: 443 - RNA, 40 nt, synthetic construct
  misc_feature 1..40: note = Synthetic
  source 1..40: mol_type = other RNA
aagccaagccgaggcccggccttcccatgtgctactagag

SEQ ID NO: 444 - RNA, 41 nt, synthetic construct; identical to SEQ ID NO: 86
  misc_feature 1..41: note = Synthetic
  source 1..41: mol_type = other RNA

SEQ ID NO: 445 - RNA, 42 nt, synthetic construct
  misc_feature 1..42: note = Synthetic
  source 1..42: mol_type = other RNA
ccaaaatgccaaagccgtagcccggccagtagcccacacgtc

SEQ ID NO: 446 - RNA, 41 nt, synthetic construct
  misc_feature 1..41: note = Synthetic
  source 1..41: mol_type = other RNA
ccaaatgccaagcccgtagcccggccagtagcccacacgtc

SEQ ID NO: 447 - RNA, 40 nt, synthetic construct; identical to SEQ ID NO: 87
  misc_feature 1..40: note = Synthetic
  source 1..40: mol_type = other RNA

SEQ ID NO: 448 - RNA, 40 nt, synthetic construct
  misc_feature 1..40: note = Synthetic
  source 1..40: mol_type = other RNA
ccattacgcgacgtaattcccccatcgtctcctcgttaag

SEQ ID NO: 449 - RNA, 40 nt, synthetic construct
  misc_feature 1..40: note = Synthetic
  source 1..40: mol_type = other RNA
ccattacgcgacgtaattcccccatcgcttcctcgttaag

SEQ ID NO: 450 - RNA, 40 nt, synthetic construct
  misc_feature 1..40: note = Synthetic
  source 1..40: mol_type = other RNA
ccattacgcggcgtaattcccccatcgtttcctcgttaag

SEQ ID NO: 451 - RNA, 40 nt, synthetic construct
  misc_feature 1..40: note = Synthetic
  source 1..40: mol_type = other RNA
ccattacgcgacgtaattcccccatcgtttcctcgttagg

SEQ ID NO: 452 - RNA, 40 nt, synthetic construct
  misc_feature 1..40: note = Synthetic
  source 1..40: mol_type = other RNA
ccattacgcgacgtaattcccccatcgtttcctcgctaag

SEQ ID NO: 453 - RNA, 40 nt, synthetic construct
  misc_feature 1..40: note = Synthetic
  source 1..40: mol_type = other RNA
ccattacgcgacgtaattcccccatcgtttcctcgttatg

SEQ ID NO: 454 - RNA, 40 nt, synthetic construct
  misc_feature 1..40: note = Synthetic
  source 1..40: mol_type = other RNA
ccattacgcgacgtaattcccccatcgtttcctcgttaaa

SEQ ID NO: 455 - RNA, 38 nt, synthetic construct; identical to SEQ ID NO: 88
  misc_feature 1..38: note = Synthetic
  source 1..38: mol_type = other RNA

SEQ ID NO: 456 - RNA, 38 nt, synthetic construct
  misc_feature 1..38: note = Synthetic
  source 1..38: mol_type = other RNA
ccatctagatctccgtagattccccagctctttctcgc

SEQ ID NO: 457 - RNA, 38 nt, synthetic construct
  misc_feature 1..38: note = Synthetic
  source 1..38: mol_type = other RNA
ccatctagatctccgtagatcccccggctctttctcgc

SEQ ID NO: 458 - RNA, 38 nt, synthetic construct
  misc_feature 1..38: note = Synthetic
  source 1..38: mol_type = other RNA
ccatctagatctccgtagattcccccgctctttctcgc

SEQ ID NO: 459 - RNA, 38 nt, synthetic construct
  misc_feature 1..38: note = Synthetic
  source 1..38: mol_type = other RNA
ccatctagatctccgtagattccccggctcttcctcgc

SEQ ID NO: 460 - RNA, 38 nt, synthetic construct
  misc_feature 1..38: note = Synthetic
  source 1..38: mol_type = other RNA
ccatctagatctccgtgattcccccggctctttctcgc

SEQ ID NO: 461 - RNA, 38 nt, synthetic construct
  misc_feature 1..38: note = Synthetic
  source 1..38: mol_type = other RNA
ccatctagatctccgtagttcccccggctctttctcgc

SEQ ID NO: 462 - RNA, 38 nt, synthetic construct
  misc_feature 1..38: note = Synthetic
  source 1..38: mol_type = other RNA
ccatctagatccccgtagattccccggctctttctcgc

SEQ ID NO: 463 - RNA, 38 nt, synthetic construct
  misc_feature 1..38: note = Synthetic
  source 1..38: mol_type = other RNA
ccatctagatctccgtagattccccggctccttctcgc

SEQ ID NO: 464 - RNA, 38 nt, synthetic construct
  misc_feature 1..38: note = Synthetic
  source 1..38: mol_type = other RNA
ccatctatatctccgtagattccccggctctttctcgc

SEQ ID NO: 465 - RNA, 40 nt, synthetic construct; identical to SEQ ID NO: 89
  misc_feature 1..40: note = Synthetic
  source 1..40: mol_type = other RNA

SEQ ID NO: 466 - RNA, 41 nt, synthetic construct
  misc_feature 1..41: note = Synthetic
  source 1..41: mol_type = other RNA
actgtctgcatacacggtatgcccaacgccatccaaaccgc

SEQ ID NO: 467 - RNA, 40 nt, synthetic construct
  misc_feature 1..40: note = Synthetic
  source 1..40: mol_type = other RNA
actgtctgcatacatggtatgcccaacgccatccaaaccg

SEQ ID NO: 468 - RNA, 41 nt, synthetic construct
  misc_feature 1..41: note = Synthetic
  source 1..41: mol_type = other RNA
actgtctgcatacacggtatgcccaacgccatccaaaaccg

SEQ ID NO: 469 - RNA, 40 nt, synthetic construct; identical to SEQ ID NO: 90
  misc_feature 1..40: note = Synthetic
  source 1..40: mol_type = other RNA

SEQ ID NO: 470 - RNA, 40 nt, synthetic construct
  misc_feature 1..40: note = Synthetic
  source 1..40: mol_type = other RNA
acctgcggctattgccagcgccataagaccctccacagca

SEQ ID NO: 471 - RNA, 39 nt, synthetic construct
  misc_feature 1..39: note = Synthetic
  source 1..39: mol_type = other RNA
cctgcggctattgccagcgccataagaccctccacagta

SEQ ID NO: 472 - RNA, 40 nt, synthetic construct
  misc_feature 1..40: note = Synthetic
  source 1..40: mol_type = other RNA
acctgcggctattgccagcgccataagaccttccacagta

SEQ ID NO: 473 - RNA, 40 nt, synthetic construct
  misc_feature 1..40: note = Synthetic
  source 1..40: mol_type = other RNA
acctgcggctattgccagcgccataagaccctccgcagta

SEQ ID NO: 474 - RNA, 56 nt, synthetic construct
  misc_feature 1..56: note = Synthetic
  source 1..56: mol_type = other RNA
ggaagagggatgggtgccagctttgcataccacgtgcaattcactccacccgtcac

SEQ ID NO: 475 - RNA, 64 nt, synthetic construct
  misc_feature 1..64: note = Synthetic
  source 1..64: mol_type = other RNA
gggagagaggaagagggatgggagccagctttgcataccacgtgcaattcactccacccgtcac

SEQ ID NO: 476 - RNA, 56 nt, synthetic construct
  misc_feature 1..56: note = Synthetic
  source 1..56: mol_type = other RNA
gggatgggcacatggtacgcccaaagcgaggcccgctgcgtagtgccataacccag

SEQ ID NO: 477 - RNA, 61 nt, synthetic construct
  misc_feature 1..61: note = Synthetic
  source 1..61: mol_type = other RNA
gggagagaggaagagggatgggcacatggtacgcccaaagcgaggcccgctgcgtagtgcc

SEQ ID NO: 478 - RNA, 66 nt, synthetic construct
  misc_feature 1..66: note = Synthetic
  source 1..66: mol_type = other RNA
gggatgggcacggtccagcgctaactgtacctgctgtgccacccaccgcataacccagaggtcgat

SEQ ID NO: 479 - RNA, 49 nt, synthetic construct
  misc_feature 1..49: note = Synthetic
  source 1..49: mol_type = other RNA
gggatgggcacggtccagcgctaactgtacctgctgtgccacccaccgc

SEQ ID NO: 480 - RNA, 62 nt, synthetic construct
  misc_feature 1..62: note = Synthetic
  source 1..62: mol_type = other RNA
gggagagaggaagagggatgggcacggtccagcgctaactgtacctgctgtgccacccaccg

SEQ ID NO: 481 - RNA, 45 nt, synthetic construct
  misc_feature 1..45: note = Synthetic
  source 1..45: mol_type = other RNA
gggagagaggaagagggatgggcacggtccagcgctaactgtacc

SEQ ID NO: 482 - RNA, 53 nt, synthetic construct
  misc_feature 1..53: note = Synthetic
  source 1..53: mol_type = other RNA
ggaagagggatgggcacggtccagcgctaactgtacctgctgtgccacccacc

SEQ ID NO: 483 - RNA, 43 nt, synthetic construct
  misc_feature 1..43: note = Synthetic
  source 1..43: mol_type = other RNA
gggaccacgcgccaacgtgtcagctacacgccgtgttccccgg

SEQ ID NO: 484 - RNA, 61 nt, synthetic construct
  misc_feature 1..61: note = Synthetic
  source 1..61: mol_type = other RNA
gggaccacgcgccaacgtgtcagctacacgccgtgttccccggcataacccagaggtcgat

SEQ ID NO: 485 - RNA, 62 nt, synthetic construct
  misc_feature 1..62: note = Synthetic
  source 1..62: mol_type = other RNA
gggagagaggaagagggatgggaccacgcgccaacgtgtcagctacacgccgtgttccccgg

SEQ ID NO: 486 - RNA, 44 nt, synthetic construct
  misc_feature 1..44: note = Synthetic
  source 1..44: mol_type = other RNA
gggatgggcacggtccagcgctaactgtacctgctgtgccaccc

SEQ ID NO: 487 - RNA, 54 nt, synthetic construct
  misc_feature 1..54: note = Synthetic
  source 1..54: mol_type = other RNA
gggaggaagagggatgggcacggtccagcgctaactgtacctgctgtgccaccc

SEQ ID NO: 488 - RNA, 52 nt, synthetic construct
  misc_feature 1..52: note = Synthetic
  source 1..52: mol_type = other RNA
gggaggaagaggatgggcacggtccagcgctaactgtacctgctgtgccacc

SEQ ID NO: 489 - RNA, 52 nt, synthetic construct
  misc_feature 1..52: note = Synthetic
  source 1..52: mol_type = other RNA
gggaggaagagggatgggcacggtccagcgcactgtacctgctgtgccaccc

SEQ ID NO: 490 - RNA, 50 nt, synthetic construct
  misc_feature 1..50: note = Synthetic
  source 1..50: mol_type = other RNA
gggaggaagaggatgggcacggtccagcgcactgtacctgctgtgccacc

SEQ ID NO: 491 - RNA, 22 nt, synthetic construct
  misc_feature 1..22: note = Synthetic
  source 1..22: mol_type = other RNA
gggagagaggaagagggatggg

SEQ ID NO: 492 - RNA, 34 nt, synthetic construct
  misc_feature 1..34: note = Synthetic
  source 1..34: mol_type = other RNA
cataacccagaggtcgatagtactggatcccccc

SEQ ID NO: 493 - protein, 710 aa, Homo sapiens
  REGION 1..710: note = misc_feature - amino acid sequence for nucleolin
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 494 - RNA, 49 nt, synthetic construct
  misc_feature 1..49: note = Synthetic
  source 1..49: mol_type = other RNA
gggatgggaagatctgctaagtgcacgcacaatcaccatcgagcgtctc

SEQ ID NO: 495 - RNA, 63 nt, synthetic construct
  misc_feature 1..63: note = Synthetic
  source 1..63: mol_type = other RNA
gggagagaggaagagggatgggaagatctgctaagtgcacgcacaatcaccatcgagcgtctc

SEQ ID NO: 496 - RNA, 54 nt, synthetic construct
  misc_feature 1..54: note = Synthetic
  source 1..54: mol_type = other RNA
gggagagaggaagagggatgggcacggtccagcgctaactgtacctgctgtgcc

SEQ ID NO: 497 - RNA, 58 nt, synthetic construct
  misc_feature 1..58: note = Synthetic
  source 1..58: mol_type = other RNA
gggagagagagggatgggcacggtccagcgctaactgtacctgctgtgccacccaccg

SEQ ID NO: 498 - RNA, 60 nt, synthetic construct
  misc_feature 1..60: note = Synthetic
  source 1..60: mol_type = other RNA
gggagagaggaagaggatgggcacggtccagcgctaactgtacctgctgtgccaccaccg

SEQ ID NO: 499 - RNA, 60 nt, synthetic construct
  misc_feature 1..60: note = Synthetic
  source 1..60: mol_type = other RNA
gggagagaggaagagggagggcacggtccagcgctaactgtacctgctgtgcccccaccg

SEQ ID NO: 500 - RNA, 52 nt, synthetic construct
  misc_feature 1..52: note = Synthetic
  source 1..52: mol_type = other RNA
gggagagaggaagagggatgggtccagcgctaactgtacctgccacccaccg

SEQ ID NO: 501 - RNA, 58 nt, synthetic construct
  misc_feature 1..58: note = Synthetic
  source 1..58: mol_type = other RNA
gggagagaggaagagggatgggcggtccagcgctaactgtacctgctgccacccaccg

SEQ ID NO: 502 - RNA, 58 nt, synthetic construct
  misc_feature 1..58: note = Synthetic
  source 1..58: mol_type = other RNA
gggagagaggaagagggatgggcacggtccagcgctatgtctgctgtgccacccaccg

SEQ ID NO: 503 - RNA, 58 nt, synthetic construct
  misc_feature 1..58: note = Synthetic
  source 1..58: mol_type = other RNA
gggagagaggaagagggatgggcacggtccagcgctaactgtacctgctgtgccaccc

SEQ ID NO: 504 - RNA, 58 nt, synthetic construct
  misc_feature 1..58: note = Synthetic
  source 1..58: mol_type = other RNA
gggaggaagagggatgggcacggtccagcgctaactgtacctgctgtgccacccaccg

SEQ ID NO: 505 - RNA, 54 nt, synthetic construct
  misc_feature 1..54: note = Synthetic
  source 1..54: mol_type = other RNA
ggaagagggatgggcacggtccagcgctaactgtacctgctgtgccacccaccg

SEQ ID NO: 506 - RNA, 58 nt, synthetic construct
  misc_feature 1..58: note = Synthetic
  source 1..58: mol_type = other RNA
gagaggaagagggatgggcacggtccagcgctaactgtacctgctgtgccacccaccg

SEQ ID NO: 507 - RNA, 60 nt, synthetic construct
  misc_feature 1..60: note = Synthetic
  source 1..60: mol_type = other RNA
gggagagaggaagagggatgggcacggtccagcgctaactgtacctgctgtgccacccac

SEQ ID NO: 508 - RNA, 60 nt, synthetic construct
  misc_feature 1..60: note = Synthetic
  source 1..60: mol_type = other RNA
gggagagaggaagagggatgggcacggtccagcgctaactgtacctgctgtgccaccccg

SEQ ID NO: 509 - RNA, 60 nt, synthetic construct
  misc_feature 1..60: note = Synthetic
  source 1..60: mol_type = other RNA
gggagagaggaagagggatgggcacggtccgcgctaactgtacctgctggccacccaccg

SEQ ID NO: 510 - RNA, 60 nt, synthetic construct
  misc_feature 1..60: note = Synthetic
  source 1..60: mol_type = other RNA
gggagagaggaagagggatgggcacggtccgcgctaactgtaccgctgtgccacccaccg

SEQ ID NO: 511 - RNA, 60 nt, synthetic construct
  misc_feature 1..60: note = Synthetic
  source 1..60: mol_type = other RNA
gggagagaggaagagggatgggcacggtccagcgcactgtacctgctgtgccacccaccg

SEQ ID NO: 512 - RNA, 62 nt, synthetic construct; identical to SEQ ID NO: 480
  misc_feature 1..62: note = Synthetic
  source 1..62: mol_type = other RNA

SEQ ID NO: 513 - RNA, 60 nt, synthetic construct
  misc_feature 1..60: note = Synthetic
  source 1..60: mol_type = other RNA
gggagaggaagagggatgggcacggtccagcgctaactgtacctgctgtgccacccaccg

SEQ ID NO: 514 - RNA, 60 nt, synthetic construct; identical to SEQ ID NO: 513
  misc_feature 1..60: note = Synthetic
  source 1..60: mol_type = other RNA

SEQ ID NO: 515 - RNA, 56 nt, synthetic construct
  misc_feature 1..56: note = Synthetic
  source 1..56: mol_type = other RNA
gaggaagagggatgggcacggtccagcgctaactgtacctgctgtgccacccaccg